(19) United States
(12) Patent Application Publication
(10) Pub. No.: US 2003/0087799 A1
(43) Pub. Date: May 8, 2003

(54) Modulation

(76) Inventors: Jakob Wolfart, Sceaux (FR); Jochen Roeper, Marburg (DE)

Correspondence Address: FROMMER LAWRENCE & HAUG, 745 FIFTH AVENUE- 10TH FL., NEW YORK, NY, 10151, US

(21) Appl. No.: 10/216,128

(22) Filed: Aug. 9, 2002

(30) Foreign Application Priority Data
Nov. 7, 2001 (GB) 0126781.4

(51) Int. Cl. A61K 31/00

(52) U.S. Cl. 514/1

(57) ABSTRACT

A method of treatment is described. The method comprises administering to a subject in need of same an agent, wherein said agent is capable of causing a dopaminergic neuron to enter bursting mode and/or of preventing it from leaving bursting mode. In a preferred aspect, the said agent modulates: a T-type channel and/or an SK (preferably SK3) channel and/or the coupling of a T-type channel with an SK (preferably SK3) channel.

### FIELD OF INVENTION

The present invention relates, inter alia, to modulation of the activity of dopaminergic neurons.

Dopaminergic neurons in the substantia nigra can switch from pacemaker mode (regular low level firing of action potentials) to bursting mode (high frequency bursts of action potential firing). When these neurons fire in bursting mode there is an elevation in dopamine release.

Despite intensive research on dopaminergic neurons, the mechanism of how dopaminergic neurons switch their firing pattern from pacemaker to bursting mode was unknown.

### INVENTION SUMMARY

In accordance with the present invention we have now identified how dopaminergic neurons switch their firing pattern from pacemaker mode to bursting mode.

In particular, we have found that preferential coupling of small-conductance, calcium-activated potassium (SK) channels to T-type calcium channels prevents bursting in dopaminergic midbrain neurons.

These findings have important implications in a number of clinical/medical fields—such as in the therapy of schizophrenia and Parkinson's disease.

### ASPECTS OF THE PRESENT INVENTION

Aspects of the present invention are presented in the accompanying claims and in the following description.

By way of example, key aspects of the present invention relate to:

Use of an agent in the manufacture of a medicament, wherein said agent is capable of causing a dopaminergic neuron to enter bursting mode and/or of preventing it from leaving bursting mode.

A method of treatment comprising administering to a subject in need of same an agent, wherein said agent is capable of causing a dopaminergic neuron to enter bursting mode and/or of preventing it from leaving bursting mode.

An assay method for identifying and/or improving the effect (such as by using the assay in a drug development program to improve efficacy) of an agent, wherein said agent is capable of causing a dopaminergic neuron to enter bursting mode and/or of preventing it from leaving bursting mode.

The present invention also encompasses a diagnostic composition or kit comprising means for detecting (directly or indirectly) whether or not a dopaminergic neuron is in or is prone to enter or remain in bursting mode.

An example of indirect means would be means to determine the level of Ca²⁺ influx and/or K⁺ efflux.

The diagnostic composition or kit may also contain an agent accoding to the present invention.

The present invention also includes a kit comprising the agent of the present invention and another pharmaceutically active agent, wherein said other agent is optionally capable of acting as an agent according to the present invention.

By way of further example, preferred aspects of the present invention relate to:

Use of an agent in the manufacture of a medicament, wherein said agent is capable of affecting (in particular causing a change in) the firing mode of a dopaminergic neuron that causes a beneficial increase in dopamine release from said dopaminergic neuron, wherein said agent blocks: a T-type channel and/or an SK (preferably SK3) channel and/or the coupling of a T-type channel with an SK (preferably SK3) channel.

A method of treatment comprising administering to a subject in need of same an agent, wherein said agent is capable of affecting (in particular causing a change in) the firing mode of a dopaminergic neuron that causes a beneficial increase in dopamine release from said dopaminergic neuron, wherein said agent blocks: a T-type channel and/or an SK (preferably SK3) channel and/or the coupling of a T-type channel with an SK (preferably SK3) channel.

An assay method for identifying and/or improving the effect of an agent, wherein said assay utilises: a T-type channel and/or an SK (preferably SK3) channel and/or the coupling of a T-type channel with an SK (preferably SK3) channel, wherein said agent is capable of affecting (in particular causing a change in) the firing mode of a dopaminergic neuron that causes a beneficial increase in dopamine release from said dopaminergic neuron, wherein said agent blocks: a T-type channel and/or an SK (preferably SK3) channel and/or the coupling of a T-type channel with an SK (preferably SK3) channel.

A process comprising the steps of:

(a) performing the assay according to the present invention;

(b) identifying one or more agents capable of modulating a T-type channel and/or an SK (preferably SK3) channel and/or the coupling of a T-type channel with an SK (preferably SK3) channel; and

(c) preparing a quantity of those one or more identified agents.

A process comprising the steps of:

(a) performing the assay according to the present invention;

(b) identifying one or more agents capable of modulating a T-type channel and/or an SK (preferably SK3) channel and/or the coupling of a T-type channel with an SK (preferably SK3) channel; and

(c) modifying one or more identified agents.

(d) performing the assay according to the present invention;

(e) checking that the one or more agents of (d) are capable of modulating a T-type channel and/or an SK (preferably SK3) channel and/or the coupling of a T-type channel with an SK (preferably SK3) channel;

(f) optionally repeating steps (c)-(e) one or more times; and

(g) preparing a quantity of the one or more identified modified agents.

A method of treating a condition that can be alleviated with an increase in dopamine levels, preferably endogeneous dopamine levels comprising administering to a subject in need of same an agent; wherein the agent is capable of modulating a T-type channel and/or an SK (preferably SK3) channel and/or the coupling of a T-type channel with an SK (preferably SK3) channel in an in vitro assay method; wherein the in vitro assay method is the assay method of the present invention.

Use of an agent in the preparation of a pharmaceutical composition for the treatment of a condition that can be alleviated with an increase in dopamine levels, preferably endogeneous dopamine levels, wherein the agent is capable of modulating a T-type channel and/or an SK (preferably SK3) channel and/or the coupling of a T-type channel with an SK (preferably SK3) channel in an in vitro assay method; wherein the in vitro assay method is the assay method of the present invention.

An agent identified by the assay method of the present invention.

A T-type channel and/or an SK (preferably SK3) channel and/or the coupling of a T-type channel with an SK (preferably SK3) channel channel for use in medicine.

A modulator of: a T-type channel and/or an SK (preferably SK3) channel and/or the coupling of a T-type channel with an SK (preferably SK3) channel channel for use in medicine.

A blocker of: a T-type channel and/or an SK (preferably SK3) channel and/or the coupling of a T-type channel with an SK (preferably SK3) channel channel for use in medicine.

Use of a T-type channel and/or an SK (preferably SK3) channel and/or the coupling of a T-type channel with an SK (preferably SK3) channel channel for use in the preparation of a medicament for the treatment of a condition that can be alleviated with an increase in dopamine levels, preferably endogeneous dopamine levels, preferably wherein the condition is Parkinson's disease.

Use of a modulator of a T-type channel and/or an SK (preferably SK3) channel and/or the coupling of a T-type channel with an SK (preferably SK3) channel channel for use in the preparation of a medicament for the treatment of a condition that can be alleviated with an increase in dopamine levels, preferably endogeneous dopamine levels, preferably wherein the condition is Parkinson's disease.

Use of a blocker of a T-type channel and/or an SK (preferably SK3) channel and/or the coupling of a T-type channel with an SK (preferably SK3) channel channel for use in the preparation of a medicament for the treatment of a condition that can be alleviated with an increase in dopamine levels, preferably endogeneous dopamine levels, preferably wherein the condition is Parkinson's disease.

Other aspects of the present invention are presented in the accompanying claims and in the following description and drawings. These aspects are presented under separate section headings. However, it is to be understood that the teachings under each section are not necessarily limited to that particular section heading.

In the following commentary, reference to “nucleotide sequence of the present invention” and “amino acid sequence of the present invention” refer respectively to any one or more nucleotide sequences presented or discussed herein and to any one or more of the amino acid sequences presented or discussed herein. Also, and as used herein, “amino acid” refers to peptide or protein sequence and may refer to portions thereof. In addition, the term “amino acid sequence of the present invention” is synonymous with the phrase “polypeptide of the present invention”. Also the term “nucleotide sequence of the present invention” is synonymous with the phrase “poly-nucleotide sequence of the present invention”.

Preferable Aspects

Preferable aspects of the present invention are presented in the accompanying claims and in the following description.

Preferably the agent indirectly causes the dopaminergic neuron to enter bursting mode.

In a more preferred embodiment, the agent indirectly causes the dopaminergic neuron to enter bursting mode by affecting T-type channel and/or an SK (preferably SK3) channel and/or the coupling of a T-type channel with an SK (preferably SK3) channel.

Preferably the agent is for the treatment of conditions that can be alleviated by an increase in dopamine levels.

Preferably the dopaminergic neuron is a mid-brain dopaminergic neuron.

Preferably the dopaminergic neuron is a dopaminergic Substantia nigra neuron.

Preferably the disorder is a neuronal disorder.

Preferably the disorder is a neurodegenerative disorder.

Preferably the disorder affects the mid-brain

Preferably the disorder affects the Substantia nigra.

Preferably the agent is for the treatment of Parkinson's disease.

Disorders to be treated may be genetic in origin. Thus, they may arise due to one or more mutations that result in a deleterious effect, e.g. mutations in genes or in other regions. utations may result in excessive, insufficient, or otherwise aberrant expression or activity of a gene product. Disorders to be treated may also/alternatively arise due to environmental factors.

In a highly preferred aspect the agent affects a dopaminergic neuron entering bursting mode and/or of preventing it from leaving bursting mode, wherein said agent modulates: a T-type channel and/or an SK3 channel and/or the coupling of a T-type channel with an SK3 channel.

Dopaminergic Neuron

Neurons are highly polarised cells whose most obvious feature is their extensive system of axons and dendrites. Neurons are cell populations that are distinct in their morphology, function and/or biochemical characteristics. As used herein the term “dopaminergic neuron” relates to neurons which have cell bodies in the region of the ventral midbrain (VM) known as the substantia nigra pars compacta and project to the striatum. These neurons are distinguished biochemically by the fact that they secrete dopamine as a neurotransmitter and thus express at high levels the enzyme tyrosine hydroxylase (TH) which catalyzes the rate-limiting step in the biosynthesis of dopamine.

Dopaminergic neurons are of enormous clinical significance because it is these cells that progressively lose function in patients with neurodegenerative disorders such as Parkinson's disease.

Dopaminergic (DA) midbrain neurones play an essential role in a variety of brain functions such as voluntary movement, working memory, and reward (Goldman-Rakic, 1999; Kitai et al., 1999; Spanagel and Weiss, 1999). In addition, they are intimately involved in neuropsychiatric and neurological disorders such as schizophrenia, drug addiction, and Parkinson's disease (Dunnett and Bjorklund, 1999; Verhoeff, 1999; Svensson, 2000). Since these brain functions and diseases are associated with anatomically distinct DA neurone subpopulations, the question arises whether the functional characteristics of mesencephalic DA neurones are differentiated in accordance to their anatomical subgroups. Within parallel mesostriatal (Maurin et al., 1999), mesolimbic (Groenewegen et al., 1999), and mesocortical networks (Tzschentke, 2001), DA neurones exert their function by integrating synaptic inputs in the context of their intrinsic pacemaker to generate pattern of electrical activity that control dopamine release and their functional effects on their respective target cells. Thus, potential diversity within the DA system might originate both from differences in axonal projections and synaptic connectivity as well as from diverging properties of the somato-dendritic integrator and pacemaker.

Dopaminergic midbrain neurones are distributed in three partially overlapping nuclei: the retrorubral area (RRA, A8), substantia nigra (SN, A9), and the ventral tegmental area (VTA, A10), which correspond to different mesotelencephalic projections (Gardner and Ashby, 2000; Joel and Weiner, 2000). Substantia nigra neurones mainly target the dorsal striatum (mesostriatal projection) and are involved in motor function, whereas the neurones of the VTA project predominantly to the ventral striatum e.g. nucleus accumbens (mesolimbic projection) and to prefrontal cortex (mesocortical projection) and are thus associated with limbic functions (Gardner and Ashby, 2000; Joel and Weiner, 2000). Recent tracing studies have revealed a more refined concept of the topographical organisation of mesotelencephalic connections. In the SN, two neurochemically distinct tiers project to and receive input from different neurochemical compartments in the striatum, namely the limbic and sensori-motor regions (Maurin et al., 1999; Haber et al., 2000). Ventral tier DA neurones that do not express the calcium-binding protein calbindin D₂₈-K (calbindin-negative, CB−), project to patch compartments and in turn receive innervation from striatal projection neurones in the matrix. Conversely, calbindin-positive (CB+) dorsal tier DA neurones project to the matrix while receiving input from the limbic patch compartment (Gerfen, 1992; Barrot et al., 2000). Several studies have revealed that the striking differences in the vulnerability of DA neurones to degeneration in both Parkinson's disease and its animal models, are tightly associated with the differential expression of CB. Thus CB+ DA neurones are less vulnerable (Liang et al., 1996; Damier et al., 1999a; Gonzalez-Hernandez and Rodriguez, 2000; Tan et al., 2000).

In contrast to the well described anatomical and neurochemical organisation much less is known about the electrophysiology within different subpopulations of DA neurones. To date, in vitro electrophysiological studies have considered DA midbrain neurones mainly as a single population (Pucak and Grace, 1994; Kitai et al., 1999). These “classical” DA neurones have a number of features suggestive of the expression of a highly conserved repertoire of ion channels namely, low-frequency pacemaker activity, broad action potentials followed by a pronounced afterhyperpolarisation, a strong sag-component mediated by hyperpolarisation activated channels (Ih channels) and a D2 autoreceptor-mediated hyperpolarisation (Sanghera et al., 1984; Grace and Onn, 1989; Lacey et al., 1989; Richards et al., 1997). However, in vivo studies have highlighted significant differences in discharge rates, burst firing, and autoreceptor control between subgroups of DA midbrain neurones (Chiodo et al., 1984; Greenhoff et al., 1988; Shepard and German, 1988; Paladini et al., 1999).

Neuron Activity

Neurons, in general, have a single axon which is specialised for the conductance of a particular type of electric impulse called an action potential. An action potential is a series of sudden changes in the electric potential across the neuron plasma membrane. The potential varies according to the physiological mode of the plasma membrane of the neuronal cell which may be resting, firing, pacemaker or bursting.

The term “resting mode” relates to a neuron plasma membrane which is non-stimulated and in which any voltage-gated ion channels, such as a T-type calcium channel or an SK (preferably SK3) potassium channel, are open.

On stimulation, in response to a change in membrane potential, the voltage-gated ion channels are open only for a fraction of a second which propagates action potentials along the neuron plasma membrane. As used herein the term “firing mode” relates to a stimulated neuron plasma membrane which is capable of generating action potentials.

As used herein the term “pacemaker mode” relates to a regular or maintained low level of firing action potentials.

As used herein the term “bursting mode” relates to a high frequency firing of action potentials.

T-Type Channels

T-type channels are so called because they carry a transient current, with a low voltage of activation and rapid inactivation. The main factor which defines the different calcium currents is which α₁ subtype is included in the channel complex. The subfamily of α_{1G}, α_{1H} and α_{1I} subunits display the low-voltage activation characteristic of T-type channels. T-type channels are located in cardiac & vascular smooth muscle; and in the nervous system. Perez-Reyes et al discuss the molecular characterization of a neuronal low-voltage-activated T-type calcium channel (Nature 391, 896-900, 1998). T-type channels may be blocked for example by nickel ions, Mibefradil (available from Roche), and Kurtoxin (a peptide from South African scorpion, Parabuthus transvaalicus).

Further background teachings on T-type calcium channels has been presented by Victor A. McKusick et al on http://www3.ncbi.nlm.nih.gov/Omim/searchomim.htm. The following information concerning T-type calcium channels has been extracted from that source.

Voltage-activated calcium channels can be distinguished based on their voltage-dependence, deactivation, and single-channel conductance. Low-voltage-activated calcium channels are referred to as ‘T’ type because their currents are both transient, owing to fast inactivation, and tiny, owing to small conductance. T-type channels are thought to be involved in pacemaker activity, low-threshold calcium spikes, neuronal oscillations and resonance, and rebound burst firing. By searching an EST database for sequences related to calcium channels Perez-Reyes et al. (1998) identified a partial human cDNA encoding a novel channel that they designated alpha-1G or Ca(V)T.1. The authors used the partial cDNA to isolate additional human, rat, and mouse alpha-1G cDNAs. Northern blot analysis of human and rat tissues indicated that the alpha-1G gene was expressed as an 8.5-kb mRNA predominantly in brain. An additional 9.7-kb transcript was also detected. When expressed in Xenopus oocytes, the rat alpha-1G channel exhibited the properties of a low-voltage-activated T-type calcium channel.

By FISH and radiation hybrid analysis, Perez-Reyes et al. (1998) mapped the CACNA1G gene to 17q22. Using interspecific backcross analysis, they mapped the mouse Cacna1g gene to the distal portion of chromosome 11, in a region showing homology of synteny with 17q22.

Native calcium channels have been classified by their electrophysiological and pharmacological properties as T, L, N, P and Q types (for views see McCleskey, E. W. et al. Curr Topics Membr (1991) 39:295-326, and Dunlap, K. et al. Trends Neurosci (1995) 18:89-98). T-type (or low voltage-activated) channels describe a broad class of molecules that transiently activate at negative potentials and are highly sensitive to changes in resting potential. The L, N, P and Q-type channels activate at more positive potentials (high voltage activated) and display diverse kinetics and voltage-dependent properties. There is some overlap in biophysical properties of the high voltage-activated channels, consequently pharmacological profiles are usefull to further distinguish them. L-type channels are sensitive to dihydropyridine agonists and antagonists, N-type channels are blocked by the Conus geographus peptide toxin, .omega.-conotoxin GVIA, and P-type channels are blocked by the peptide aagatoxm IVA from the venom of the funnel web spider, Agelenopsis aperta. A fourth type of high voltage-activated calcium channel (Q-type) has been described, although whether the Q- and P-type channels are distinct molecular entities is controversial (Sather, W. A et al. Neuron (1995) 11:291-303; Stea, A. et al. Proc Natl Acad Sci USA (1994) 91:10576-10580; Bourinet, E. et al. Nature Neuroscience (1999) 2:407415). Several types of calcium conductances do not fall neatly into any of the above categories and there is variability of properties even within a category suggesting that additional calcium channels subtypes remain to be classified. Biochemical analyses show that neuronal high voltage activated calcium channels are hetero oligomeric complexes consisting of three distinct subunits (.alpha.₁, .alpha.₂.delta. and β) (reviewed by De Waard, M. et at. Ion Channels (1997) vol. 4, Narahashi, T. ed. Plenum Press, NY). The .alpha.₁ subunit is the major pore-forming subunit and contains the voltage sensor and binding sites for calcium channel antagonists. The mainly extracellular .alpha.₂ is disulfide-linked to the transmembrane delta. subunit and both are derived from the same gene and are proteolytically cleaved in vivo. The β subunit is a nonglycosylated, hydrophilic protein with a high affinity of binding to a cytoplasmic region of the alpha.₁ subunit. A fourth subunit, .gamma., is unique to L-type calcium channels expressed in skeletal muscle T-tubules. The isolation and characterization of .gamma.-subunit-encoding cDNAs is described in U.S. Pat. No. 5,386,025

Recently, each of these .alpha.₁ subtypes has been cloned and expressed, thus permitting more extensive pharmacological studies. These channels have been designated alpha._{A}-.alpha._{1I} and .alpha._{1S} and correlated with the subtypes set forth above. alpha._{1A} channel s are of the P/Q type; alpha._{1B} represents N; .alpha._{1C}, .alpha.′_{1D}, .alpha._{1F} and acs represent L; .alpha._{1E} represents a novel type of calcium conductance, and .alpha._{1G-}.alpha._{1I} represent members of the T-type family, reviewed in Stea, A. et al. in Handbook of Receptors and Channels (1994), North, R. A. ed. CRC Press; Perez-Reyes, et al. Nature (1998) 391:896-900; Cribbs, L. L. et al. Circulation Research (1998) 83:103-109; Lee, J. H. et al. Journal of Neuroscience (1999) 19:1912-1921.

SK Channels

SK channels are activated in a voltage-independent manner and have a relatively small unit conductance and high sensitivity to calcium. Kohler et al (Science 273 1709-1714, 1996) isolated rat and human brain cDNAs encoding a family of SK channels which they designated SK1, SK2, and SK3. All 3 proteins contain intracellular N and C termini and 6 highly conserved transmembrane segments. In situ hybridization revealed that mRNAs encoding these subunits are widely expressed in rat brain with distinct but overlapping patterns. Chandy et al (Molec. Psychiat. 3: 32-37, 1998) identified cDNAs encoding the human SK3, or SKCa3, homolog. The predicted protein is 731 amino acids long. The authors determined that the SK3 gene contains 2 arrays of CAG trinucleotide (polyglutamine) repeats in the N-terminal region of the protein. The second CAG repeat was highly polymorphic in control individuals, with alleles ranging in size from 12 to 28 repeats.

Further background teachings on SK3 potassium channels has been presented by Victor A. McKusick et al on http://www3.ncbi.nlm.nih.gov/Omim/searchomim.htm. The following information concerning SK3 potassium channels has been extracted from that source.

Action potentials in vertebrate neurons are followed by an afterhyperpolarization (AHP) that may persist for several seconds and may have profound consequences for the firing pattern of the neuron. Each component of the AHP is kinetically distinct and is mediated by different calcium-activated potassium channels. SK channels are activated in a voltage-independent manner and have a relatively small unit conductance and high sensitivity to calcium. Kohler et al. (1996) isolated rat and human brain cDNAs encoding a family of SK channels which they designated SKI (KCNN1), SK2 and SK3. All 3 proteins contain intracellular N and C termini and 6 highly conserved transmembrane segments. In situ hybridization revealed that mRNAs encoding these subunits are widely expressed in rat brain with distinct but overlapping patterns.

Chandy et al. (1998) identified cDNAs encoding the human SK3, or SKCa3, homolog. The predicted protein is 731 amino acids long. The authors determined that the SK3 gene contains 2 arrays of CAG trinucleotide (polyglutamine) repeats in the N-terminal region of the protein. The second CAG repeat was highly polymorphic in control individuals, with alleles ranging in size from 12 to 28 repeats. Several human hereditary neurodegenerative diseases, such as Huntington disease, are caused by expanded trinucleotide repeats within genes. Citing previous reports of expanded CAG arrays in patients with schizophrenia and bipolar disorder I. Chandy et al. (1998) tested for an association between the longer alleles of SK3 and these neuropsychiatric disorders. They found a statistically significant overrepresentation of longer alleles in schizophrenia patients and a similar, nonsignificant, trend in bipolar disorder patients, providing evidence for a possible association between longer alleles and the diseases. The authors suggested that mild variations in the length of the polyglutamine repeats might produce subtle alterations in channel function, and therefore in neuronal behavior.

Wittekindt et al. (1998) showed that both of the previously described tandemly arranged CAG repeats are located in exon 1. Homology to the previously localized sequence tagged site (STS) G16005 indicated that the gene may be on 22q; however, using PCR amplification of somatic cell hybrid DNA and fluorescence in situ hybridization (FISH) of 2 P1 artificial chromosome clones, Wittekindt et al. (1998) localized the gene physically to 1q21.3.

Having previously found an association between the highly polymorphic second (more 3-prime) CAG repeat of the KCNN3 gene and schizophrenia in 98 patients compared with 117 controls, Wittekindt et al. (1998) genotyped an additional 19 patients with schizophrenia and performed statistical analyses on the entire group of patients and controls to investigate the possible effect on the age of onset, family history, and gender of the patients on the observed association. None of these factors was found to influence the results. There was no significant difference in allele frequency of both CAG repeats found in 86 bipolar I disorder patients and controls.

Navon et al. (1998) used FISH to localize the KCNN3 gene to 1q21, noting also the alignment of an STS previously mapped to this region with the KCNN3 gene. They observed an association with the larger CAG repeat within this gene in Israeli Jewish schizophrenia patients compared to controls.

Frebourg et al. (1998) found no evidence for association between the expanded allele and schizophrenia in 20 families with clinical evidence for anticipation or in 58 sporadic schizophrenia patients.

Austin et al. (1999) mapped this gene, which they referred to as human KCa3 (hKCa3), to 1q21 by radiation hybrid analysis. In the families from the National Institute of Mental Health (NIMH) Schizophrenia Genetics Initiative, they compared transmission to discordant sibs and parental transmission to affected offspring. Overall, there was no convincing evidence that KCNN3 CAG lengths differed between schizophrenics and controls. There was also no evidence of excessive parental transmission of long CAG repeat alleles to affected offspring.

Bond et al. (2000) targeted the SK3 gene by homologous recombination for the insertion of a gene switch that permitted experimental regulation of SK3 expression while retaining normal SK3 promoter function. An absence of SK3 did not present overt phenotypic consequences. However, SK3 overexpression induced abnormal respiratory responses to hypoxia and compromised parturition, presumably by effects on uterine contraction. Both conditions were corrected by silencing the gene. Bond et al. (2000) concluded that their results implicate SK3 channels as potential therapeutic targets for disorders such as sleep apnea or sudden infant death syndrome and for regulating uterine contractions during labor.

Modulates/Modulating

As used herein the terms “modulates”/“modulating” preferably mean any one or more of: adversely affecting, decreasing, removing, inhibiting, antagonising, blocking or down regulating T-type channel activity and/or SK (preferably SK3) channel activity and/or the coupling thereof.

Blocks/Blocking

In a preferred aspect, the agent blocks T-type channel activity and/or SK3 channel activity and/or the coupling thereof. As used herein the terms “blocks”/“blocking” include partial or complete blocking. In one preferred aspect, the terms preferably mean at least substantially blocks.

The agent may be capable of reducing the level or rate of ion transport through an ion channel (especially of a calcium or potassium ion channel). Thus, agents include, but are not limited to, agents capable of causing an open ion channel to become closed so that no more ions of a given type pass through the channel when the channel is in the closed state.

By blocking either the T-type calcium channel or the SK (preferably SK3) potassium channel (or both together) success has now been achieved by the present inventors in causing the cells to switch from pacemaker to bursting mode. This was completely unexpected, because prior to the present invention it was thought that activity of the calcium channel led to bursting. However the present inventors have shown that uses the opposite is true for dopaminergic neurons—i.e. that activity of the calcium channel inhibits bursting. This leads to important medical/clinical applications as discussed below.

Dopamine

Neural transmitters are chemicals in the brain that are used to send messages from one brain cell to another. Neurotransmitters bind to special receptor proteins in the membranes of nerve cells, like a lock in a key, triggering a chemical reaction within the cell. Dopamine is an example of a central nervous system (CNS) neurotransmitter which is a catecholamine belonging to a class of biogenic amine neurotransmitters, along with norepinephrine, serotonin, and histamine. The catecholomines (particularly dopamine and serotonin) are involved in the control of movement; mood; attention; and possibly, certain endocrine, cardiovascular, and stress responses. Imbalances in neurotransmitter production have been implicated in a variety of mental and physical disorders, such as Parkinson's disease (PD). It is thus desirable to diagnose and monitor such imbalances and to monitor the effectiveness of drugs and substances that affect brain chemistry.

Dopamine is a neurotransmitter that participates in a variety of different functions mediated by the nervous system, including vision, movement, and behavior (see generally Cooper et al., 1978, The Biochemical Basis of Neuropharmacology, 3d ed., Oxford University Press, New York, pp, 161-195). The diverse physiological actions of dopamine are in turn mediated by its interaction with five of the basic types of G protein-coupled receptors, D1, D2, D3, D4 and D5 which respectively stimulate and inhibit the enzyme adenylyl cyclase (Kebabian & Calne, 1979, Nature 277: 93-96). Alterations in the number or activity of these receptors may be a contributory factor in disease states such as Parkinson's disease (a movement disorder) and schizophrenia (a behavioral disorder).

Dopamine Release

In one aspect the present invention provides the use of an agent capable of affecting (in particular causing a change in) the firing mode of a dopaminergic neuron, or of preventing a change that would otherwise occur, in the manufacture of a medicament for treating a disorder that is alleviated by a change in dopamine release.

The change in dopamine release may be an increase or decrease in dopamine release, relative to what would occur in the absence of the agent.

Preferably there is an absolute increase or decrease in dopamine release (e.g. an increase or decrease of at least 5%, 10%, 25% or 50%). Desirably this may be achieved over a sustained period—e.g. over at least 1 hour, or at least 24 hours.

Switching

According to one aspect of the present invention there is provided the use of an agent capable of causing a dopaminergic neuron to enter bursting mode and/or of preventing a dopaminergic neuron from leaving bursting mode, in the manufacture of a medicament for treating a disorder that is alleviated by increased dopamine release.

Desirably the agent causes a switch from a non-bursting mode (e.g. pacemaker mode) to bursting mode.

The present invention further provides the use of an agent capable of reducing calcium efflux through a calcium channel of a dopaminergic neuron and/or of reducing potassium efflux through a potassium channel of a dopaminergic neuron in the manufacture of a medicament for treating a disorder that is alleviated by increased dopamine release.

Preferably the agent acts by reducing calcium efflux through a T-type calcium channel and/or by reducing potassium efflux through an SK3 potassium channel.

The calcium and potassium channels are preferably channels of a dopaminergic neuron, more preferably of a Substantia nigra dopaminergic neuron.

Various disorders can be alleviated (either directly or indirectly) by increased dopamine release from the aforesaid neurons.

They include neurodegenerative disorders, such as Parkinson's Disease.

If desired, a combination of an agent acting to reduce calcium efflux through a T-type calcium channel and of another agent acting to reduce potassium efflux through an SK (preferably SK3) potassium channel may be used. The agents may be administered via a single composition or may be administered separately (e.g. sequentially). They may be provided in a kit and may be in separate containers.

In some cases, following administration of one or more agents agent causing increased dopamine release from dopaminergic neurons, it may be found that dopamine levels are undesirably high or likely to become so. An agent causing decreased dopamine release may then be provided. A kit may be provided comprising, in separate containers, both an agent capable of causing increased dopamine release and an agent capable of causing reduced dopamine release. This can be useful in regulating dopamine levels.

Disorders

Examples of disorders include stroke, chronic and acute pain, cardiac conditions such as hypertension and cardiac arrhythmias and neurodegenerative disorders.

Neurodegenerative Disorders

In a preferred aspect of the present invention the agent is used to treat a neurodegenerative disorder. Examples of neurodegenerative disorders include Parkinson's disease, Schizophrenia, Huntingdon's disease, Gilles de la Tourette syndrome, Lesch-Nyham syndrome, migraine, epilepsy, psychoses, depression, apnea and Alzheimer's disease.

Preferably the disorder is Parkinson's disease or Schizophrenia.

More preferably the disorder is Parkinson's disease.

Parkinson's Disease

Parkinson's disease is characterized by a progressive degeneration of the dopaminergic nigrostriatal pathways in the brain. Parkinsons Disease is a disturbance of voluntary movement in which muscles become stiff and sluggish, movement becomes clumsy and difficult and uncontrollable rhythmic twitching of groups of muscles produces characteristic shaking or tremor. The condition is believed to be caused by a degeneration of pre-synaptic dopaminergic neurones in the brain. The absence of adequate release of the chemical transmitter dopamine during neuronal activity thereby leads to the Parkinsonian symptomatology. One of the most widely used treatments for Parkinsonism is administration of L-DOPA, a precursor of dopamine which acts indirectly by replacing the missing dopamine. An alternative form of therapy is to administer postsynaptic dopamine agonists, for example ergot alkaloids such as bromocriptine.

Biochemical studies carried out in the 1960's lead to the discovery of the fundamental role played in the pathogenesis of this disease by the deficit of neurotransmitters, and particularly of dopamine. This important advance in the comprehension of the neurochemical bases of the pathology brought about a drastic change in the pharmacological approach to the disease, and lead to the introduction in the therapy of an immediate biologic precursor of dopamine, (−)-3-(3,4-dihydroxyphenyl)-L-alanine, more commonly known as levodopa. The use of levodopa lead to a dramatic improvement in the treatment of parkinsonism and is widely used in the treatment of this disease.

Schizophrenia

Schizophrenia is a serious disease affecting one percent of the entire global population including about three million Americans. The annual cost of this disorder to the United Sates alone due to loss of employment, hospitalizations, medications, and the like exceeds 60 billion dollars annually and its toll in human suffering is shown by the ten to thirteen percent suicide rate for people who have the disease (American Psychiatric Association Public Information Online [1998] http://www.psych.org). The symptoms of schizophrenia can be grouped into three separate categories. These are (1) positive symptoms related to hallucinations and reality distortion; (2) disorganized symptoms characterized by attentional impairment and thought disorder; and (3) negative symptoms such as apathy and loss of verbal fluency (O'Donnell, P. O. and Grace, A. A. [1998], “Dysfunctions in multiple interrelated systems as the neurobiological bases of schizophrenic symptom clusters,” Schizophrenia Bull., 24(2):267-283). A long history of research has demonstrated the efficacy of D2 receptor antagonism in the alleviation of positive and disorganized symptoms (Gray, J. A. [1998], “Integrating schizophrenia,” Schizophrenia Bull., 24(2): 249-266). Persistence of negative symptoms often continues, even following neuroleptic treatment (Arndt, S. et al. [1995], “A longitudinal study of symptom dimensions in schizophrenia,” Arch. Gen. Psychiatry, 52:352-359). The stability of negative symptoms has been, by some, attributed to the neuroleptic medications themselves (Carpenter, W. T. [1997], “The risk of medication-free research,” Schizophrenia Bull., 23(1): 11-18). Dysfunction of the limbic-cortical system may be implicated in all three types of symptoms. Reduced excitory glutamatergic inputs from the hippocampus and other limbic structures to the ventral striatum may be implicated in positive symptoms of psychosis and thought disorganization, and negative symptoms are likely to result from abnormal functioning of frontal lobe structures, e.g. those that receive connections from limbic structures, and/or anatomical irregularities (Csemansky, J. G. and Bardgett, M. E. [1998], “Limbic-Cortical Neuronal Damage and the Pathophysiology of Schizophrenia,” Schizophrenia Bull. 24(2):231-248).

Excess dopamine production is implicated in schizophrenia. The dopamine hypothesis of schizophrenia associates the disease with increased activity in dopaminergic neurons. Schizophrenic symptoms may be caused by an abnormal dopaminergic state brought about by a primary limbic-cortical lesion and deficits in glutamatergic inputs to the ventral striatum. (Csemansky, J. G. and Bardgett, M. E. [1998], supra.). Radiotracer studies have shown elevated D2 dopamine receptor levels in schizophrenic patients with increases in striatal dopamine receptors sometimes many times increased over normal values. (Seeman, P. et al. [1993], “Dopamine D2 receptors elevated in schizophrenia,” Nature, 365:441-445; Tune, L. E. et al. [1993], “Dopamine D2 Receptor Density Estimates in Schizophrenia: A Positron Emission Tomography Study with¹¹ C-N-Methylspiperone,” Psychiatry Research 49:219-237.) Pharmacologically-invoked dopamine release is estimated to be 300% higher than normal levels. (Breier, A. et al. [1997], “Schizophrenia is associated with elevated amphetamine-induced synaptic dopamine concentration: evidence from a novel positron emission tomography method,” Proc. Nat'l Acad. Sci., 94(6):2569-2574.) Dopamine projections from the substantia nigra modulate striatal neuronal activity via dopamine D1 and D2 receptors. (Egan et al. [1997], “Treatment of Tardive Dyskinesia,” Schizophrenia Bull. 23(4):583-609).

One of the strongest pieces of evidence for a dopamine disturbance in schizophrenia arises from the ability of D2 receptor antagonists to alleviate schizophrenic symptoms. Effective antipsychotics acting on D2 receptors, including “typical” antipsychotics such as haloperidol and “a typical” antipsychotics such as clozapine, result in disruptions of the dopamine system. Long-term haloperidol treatment reduces the activity of dopamine cells in the substantia nigra. Clozapine reduces the activity of dopamine cells in mesolimbic/mesocortical cells in the ventral tegmental area that projects to the limbic system. (O'Donnell, P. and Grace, A. A. [1998], “Dysfunctions in Multiple Interrelated Systems as the Neurobiological Bases of Schizophrenic Symptom Clusters,” Schizophrenia Bull. 24(2):267-284.)

Past research has demonstrated a prominent role for dopamine and D2 receptors in the manifestation of psychosis, progression and complications of this disorder. More recent research has uncovered a multitude of abnormalities of the dopamine system itself and in its relation to other neurotransmitter systems in schizophrenia. A review of these studies will convey a general understanding of other more subtle symptoms involved in schizophrenia which manifest from excessive stimulation of other than D2 dopamine receptors.

The five distinct dopamine receptors have been clustered into two families: the D1-like dopamine receptors consist of the D1 and D5 receptors; and the D2-like dopamine receptors consist of the D2, D3 and D4 receptors, the latter having high affinities for a number of antipsychotic drugs. (Damask, S. P. et al. [1996], “Differential effects of clozapine and haloperidol on dopamine receptor mRNA expression in rat striatum and cortex,” Molecular Brain Res. 41:241-249.) D4 receptors have been found to be elevated in schizophrenia. (Seeman, P. et al. [1993], “Dopamine D4 receptors elevated in schizophrenia,” Nature 365:441-445.) The “typical” antipsychotics that are highly effective in reducing hallucinations and delusions are selective antagonists of D2 receptors. The “a typical” antipsychotics, to which negative symptoms such as affective flattening, and lack of motivation respond, show affinity for both D1 and D2 receptors. (Swerdlow, Neal R. and Geyer, Mark A., “Using an Animal Model of Deficient Sensorimotor Gating to Study the Pathophysiology and New Treatments of Schizophrenia,” Schizophrenia Bulletin 24(2):285-301; Benes, F. M., “Model Generation and Testing to Probe Neural Circuitry in the Cingulate Cortex of Postmortem Schizophrenic Brain,” Schizophrenia Bull. 24(2):219-230.) The D₁ receptor is broadly distributed, while the D₅ receptor is restricted to expression in the hippocampus, thalamus and hypothalamus in the rodent. D₂, D₃ and D₄ have high affinities for dopaminergic antagonist drugs. The D₂ receptor appears to be expressed in most dopaminoceptive regions of the brain including motor and limbic structures. The D₃ and D₄ receptors are enriched in subcortical limbic system components.

Schizophrenia is treated chiefly with dopamine antagonists.

Targets

In one aspect of the present invention, a T-type channel and/or an SK (preferably SK3) channel may be used as a target in screens to identify agents capable of modulating T-type channel and/or an SK (preferably SK3) channel activity and/or the coupling thereof. By way of example, an T-type channel and/or an SK (preferably SK3) channel activity may be used as a target in screens to identify agents capable of modulating T-type channel and/or an SK (preferably SK3) channel activity and/or the coupling thereof such as, for example, decreasing the Ca²⁺ influx and/or the K⁺ efflux.

For convenience, the term “target” as used herein includes a reference to a T-type channel and/or an SK (preferably SK3) channel and/or the coupling thereof.

In this regard, the target may comprise known amino acid sequences or known nucleotide sequence encoding same or a variant, homolgue, derivative or fragment thereof which is prepared by recombinant and/or synthetic means or an expression entity comprising same.

The target may be in an isolated form and/or a purified form. The target may be present in or on a cell or a tissue.

Agent

The agent of the present invention is capable of affecting (in particular causing a change in) the firing mode of a dopaminergic neuron, or of preventing a change that would otherwise occur.

For some embodiments the agent is capable of causing a dopaminergic neuron to enter bursting mode and/or of preventing it from leaving bursting mode, in the manufacture of a medicament for treating a disorder that is alleviated by increased dopamine release.

For some embodiments the agent is capable of affecting (in particular causing a change of) calcium efflux through a calcium channel of a dopaminergic neuron and/or of potassium efflux through a potassium channel of a dopaminergic neuron in the manufacture of a medicament for treating a disorder that is alleviated by a change in dopamine release.

For some embodiments the agent is capable of reducing calcium efflux through a calcium channel of a dopaminergic neuron and/or of reducing potassium efflux through a potassium channel of a dopaminergic neuron in the manufacture of a medicament for treating a disorder that is alleviated by increased dopamine release.

Preferably the calcium channel is a T-type calcium channel and/or the potassium channel is an SK (preferably SK3) potassium channel.

As used herein, the term “agent” includes any entity capable of modulating a T-type channel and/or an SK (preferably SK3) channel activity and/or the coupling thereof By way of example, the agent of the present invention can include but is not limited to a blocker of modulating T-type channel and/or an SK (preferably SK3) channel activity and/or the coupling thereof. The agent may also be an antagonist.

If desired a combination of an agent acting to increase calcium efflux through a T-type calcium channel and of another agent acting to increase potassium efflux through an SK (preferably SK3) potassium channel can be used, although this is not essential. The agents may administered via a single composition or may be administered separately (e.g. sequentially). They may be provided in a kit and may be in separate containers.

As used herein, the term “agent” includes, but is not limited to, a compound which may be obtainable from or produced by any suitable source, whether natural or not.

The agent may be a polypeptide, such as one that has been identified by screening. If so, then a skilled person will appreciate a wide range of additional agents can be provided that may also useful. These include variants of the polypeptide having one or more amino acid changes relative to said polypeptide (including fragments); nucleic acids encoding such a polypeptide or variant, vectors or cells comprising such a nucleic acid; and agents capable of increasing expression or activity of the polypeptide or variant.

The agent may be designed or obtained from a library of compounds which may comprise peptides, as well as other compounds, such as small organic molecules and particularly new lead compounds. By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetics, a derivatised agent, a peptide cleaved from a whole protein, or a peptides synthesised synthetically (such as, by way of example, either using a peptide synthesizer or by recombinant techniques or combinations thereof, a recombinant agent, an antibody, a natural or a non-natural agent, a fusion protein or equivalent thereof and mutants, derivatives or combinations thereof. The agent may even be an a T-type channel and/or an SK (preferably SK3) channel activity and/or a mimetic of the coupling thereof or an amino acid sequence comprising same or a nucleotide sequence encoding same or a variant, homologue or derivative thereof or a functional equivalent thereof (such as a mimetic) or a combination of agents as outlined above.

The agent of the present invention may also be capable of displaying one or more other beneficial functional properties.

Preferably the agent may selectively agonise, and/or selectively upregulate or selectively inhibit a suitable target.

For some applications, preferably the agent has an EC₅₀ value of less than 300 nM, 250 nM, 200 nM, 150 nM, preferably less than about 100 nM, preferably less than about 75 nM, preferably less than about 50 nM, preferably less than about 25 nM, preferably less than about 20 nM, preferably less than about 15 nM, preferably less than about 10 nM, preferably less than about 5 nM.

For some applications, preferably the agent has at least about a 25, 50, 75, 100 fold selectivity to the desired target, preferably at least about a 150 fold selectivity to the desired target, preferably at least about a 200 fold selectivity to the desired target, preferably at least about a 250 fold selectivity to the desired target, preferably at least about a 300 fold selectivity to the desired target, preferably at least about a 350 fold selectivity to the desired target.

As used herein, the term “agent” may be a single entity or it may be a combination of agents. The agent can be an amino acid sequence or a chemical derivative thereof. The substance may even be an organic compound or other chemical. The agent may even be a nucleotide sequence—which may be a sense sequence or an anti-sense sequence. The agent may even be an antibody.

If the agent is an organic compound then for some applications that organic compound will typically comprise one or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen.

The agent may contain halo groups. Here, “halo” means fluoro, chloro, bromo or iodo.

The agent may contain one or more of alkyl, alkoxy, alkenyl, alkylene and alkenylene groups—which may be unbranched- or branched-chain.

An agent may act directly or indirectly to provide a desired effect. For example, a cell, vector or nucleic acid may be provided to a patient in order to increase levels of a desired polypeptide (including providing a polypeptide that is not present in the patient). The polypeptide may itself provide the desired effect or may act upon one or more other moieties to provide the desired effect.

In some cases an agent may act by inactivating a gene or of preventing or reducing expression at the RNA or polypeptide level. For example “knock-out” techniques may be used to render certain genes non-functional. Antisense techniques may be used to block RNA production or translation.

In other cases an agent may act by activating a gene already present in an individual (e.g. by providing a suitable promoter, enhancer or other regulatory region), by increasing expression at the RNA or polypeptide level, or by introducing an additional gene or a gene product thereof. It will therefore be appreciated that a wide range of agents can be used. Some of them may be introduced by techniques involving gene therapy.

Gene therapy techniques include introducing a nucleic acid into a patient by any appropriate means. A nucleic acid may be included in a cell or vector (e.g. a retroviral or non-retroviral vector), although this is not essential. It may be used to combine with nucleic acid in a host (e.g. via homologous or non-homologous recombination) or may remain separate from the host nucleic acid (e.g. as an episome). Gene therapy techniques are disclosed, for example, in U.S. Pat. No. 5,399,346, in WO93/09222, in U.S. Pat. No. 5,371,015, etc. Of course, non gene-therapy techniques may be used and may often be preferred.

An agent of the present invention may be provided in substantially pure or substantially isolated form. It may be provided in the form of a pharmaceutically acceptable composition—e.g admixed with a suitable pharmaceutically acceptable carrier, diluent, or excipient.

Agents that can be used to provide increased dopamine release include agents capable of causing a dopaminergic neuron to enter bursting mode and/or of preventing it from leaving bursting mode.

They include calcium and/or potassium channel blockers, preferably blockers of T-type calcium channels and/or of SK (preferably SK3) potassium channels, most preferably blockers of such channels in the Substantia nigra.

Various T-type calcium channel blockers and/or SK (preferably SK3) channel blockers can be identified by screening, as described later.

Various T-type calcium channel blockers are known—such as Mibefradil and/or nickel ions.

Other types of putative calcium channel blockers are available. Examples of which are typically dihydropyridines. Examples of dihydropyridines include nifedipine, nitrendipine, nicardipine, nimodipine, niludipine, riodipine (ryosidine) felodipine, darodipine, isradipine, (+)Bay K 8644, (−)202-791, (+)H 160/Sl, PN 200-110 and nisoldipine. Other examples of the calcium channel blocker include Kurtoxin, benzothiazepine, such as diltiazem (dilzem) and TA 3090 and phenylalkylamine, such as verapamil (isoptin), desmethoxyverapamil, methoxy verapamil (D-600, gallopamil or (−)D-888), prenylamine, fendiline, terodiline, caroverine, perhexiline.

Background teachings on calcium channel blockers has been presented by Victor A. McKusick et al on http://www3.ncbi.nlm.nih.gov/Omim/searchomim.htm. The following information concerning calcium channel blockers has been extracted from that source.

U.S. Pat. No. 5,646,149 describes calcium antagonists of the formula A-Y-B wherein B contains a pipelazine or piperidine ring directly linked to Y. An essential component of these molecules is represented by A, which must be an antioxidant; the pipeazine or piperidine itself is said to be important. The exemplified compounds contain a benzhydril substituent, based on known calcium channel blockers (see below). U.S. Pat. No. 5,703,071 discloses compounds said to be useful in treating ischemic diseases. A mandatory portion of the molecule is a tropolone residue; among the substituents permitted are piperazine derivatives, including their benzhydril derivatives. U.S. Pat. No. 5,428,038 discloses compounds which are said to exert a neural protective and antiallergic effect. These compounds are coumarin dervatives which may include derivatives of piperazine and other six-membered heterocycles. A permitted substituent on the heterocycle is diphenylhydroxymethyl. Thus, approaches in the art for various indications which may involve calcium channel blocking activity have employed compounds which incidentally contain piperidine or piperazine moieties substituted with benzhydril but mandate additional substituents to mantain functionality.

Certain compounds containing both benzhydril moieties and piperidine or piperazine are known to be calcium channel antagonists and neuroleptic drugs. For example, Gould, R. J. et al. Proc Natl Acad Sci USA (1983) 80:5122-5125 describes antischizophrenic neuroleptic drugs such as lidoflaine, fluspirilene, pimozide, clopimozide, and penfluridol. It has also been that fluspirilene binds to sites on L-type calcium channels (King, V. K. et al. J Biol Chem (1989) 264:5633-5641) as well as blockdng N-type calcium cuerent (Grantn C. J. et al. Brit J Pharmacol (1944) 111:483-488). In addition, lomerizine, as marketed by Kenebo K K, is a known calcium channel blocker. A review of publications concerning lomeriine is found in Dooley, D., Current Opinion in CPNS Investigational Drugs (1999)

Pharmaceutically Acceptable Salt

The agent may be in the form of—and/or may be administered as—a pharmaceutically acceptable salt—such as an acid addition salt or a base salt—or a solvate thereof, including a hydrate thereof. For a review on suitable salts see Berge et al, J. Pharm. Sci., 1977, 66, 1-19.

Typically, a pharmaceutically acceptable salt may be readily prepared by using a desired acid or base, as appropriate. The salt may precipitate from solution and be collected by filtration or may be recovered by evaporation of the solvent.

Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts.

Suitable base salts are formed from bases which form non-toxic salts and examples are the sodium, potassium, aluminium, calcium, magnesium, zinc and diethanolamine salts.

Polymorphic Form(s)/Asymmetric Carbon(s)

The agent of the present invention may exist in polymorphic form.

The agent of the present invention may contain one or more asymmetric carbon atoms and therefore exists in two or more stereoisomeric forms. Where an agent contains an alkenyl or alkenylene group, cis (E) and trans (Z) isomerism may also occur. The present invention includes the individual stereoisomers of the agent and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the agent or a suitable salt or derivative thereof. An individual enantiomer of a compound of the agent may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

Isotopic Variations

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, 35S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

Pro-Drug

It will be appreciated by those skilled in the art that the agent of the present invention may be derived from a prodrug. Examples of prodrugs include entities that have certain protected group(s) and which may not possess pharmacological activity as such, but may, in certain instances, be administered (such as orally or parenterally) and thereafter metabolised in the body to form the agent of the present invention which are pharmacologically active.

Pro-Moiety

It will be further appreciated that certain moieties known as “pro-moieties”, for example as described in “Design of Prodrugs” by H. Bundgaard, Elsevier, 1985 (the disclosure of which is hereby incorporated by reference), may be placed on appropriate functionalities of the agents. Such prodrugs are also included within the scope of the invention.

Antagonist

In one embodiment of the present invention, preferably the agent is selected from the group consisting of an antagonist, a partial antagonist and a competitive antagonist of a T-type channel and/or an SK (preferably SK3) channel and/or the coupling thereof.

An antagonist of a given moiety may inhibit one or more activities of that moiety (e.g. all activities). It may, for example, bind in a competitive or non-competitive manner to the moiety or to something with which the moiety interacts (e.g. binds).

Binding Agents

The agent may be associated with or may be a binding agent.

Here, binding agents may be useful as channel blockers in accordance with the present invention. For example they may block a channel by binding to a part thereof or to another moiety operatively associated with the channel.

An example of a binding agent is an antibody or a fragment thereof that is specific for a T-type channel and/or an SK (preferably SK3) channel.

A further type of binding agent that can be used in the present invention is a lectin. Lectins are carbohydrate-binding proteins of non-immune (e.g. plant) origin (see e.g. the discussion of lectins by Deutscher in Methods in Enzymology, Guide to Protein Purification, 182 (1990)). Different lectins can be used to select particular glycoproteins based upon the presence of particular carbohydrate moieties (e.g. sialic acid, galactose, mannose, fucose, N-acetyl glucosamine, N-acetyl galactosamine, etc). In some cases a plurality of different lectins may be used—e.g. if a glycoprotein is known to include three different sugars, then three different lectins may be used to purify it. They may be used sequentially (e.g. in sequential affinity columns).

Another type of binding agent is a ligand or a part thereof that binds to a polypeptide of the present invention or to a moiety with which said polypeptide interacts (e.g. binds). The ligand or part thereof may be provided in immobilised or non-immobilised (soluble) form.

It will be appreciated that many different types of binding agent can be used in the present invention.

Antibodies

In one embodiment of the present invention, the agent of the present invention may be an antibody. In addition, or in the alternative, the target of the present invention may be an antibody.

Antibodies may be produced by standard techniques, such as by immunisation with the substance of the invention or by using a phage display library.

For the purposes of this invention, the term “antibody”, unless specified to the contrary, includes but is not limited to, polyclonal, monoclonal, chimeric, single chain, Fab fragments and fragments produced by a Fab expression library. Such fragments include fragments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. Furthermore, the antibodies and fragments thereof may be humanised antibodies. Neutralizing antibodies, i.e., those which inhibit biological activity of the substance polypeptides, are especially preferred for diagnostics and therapeutics.

If polyclonal antibodies are desired, a selected mammal (e.g., mouse, rabbit, goat, horse, etc.) is immunised with an immunogenic polypeptide bearing a epitope(s) obtainable from an identified agent and/or substance of the present invention. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance polypeptide is administered to immunologically compromised individuals for the purpose of stimulating systemic defence.

Serum from the immunised animal is collected and treated according to known procedures. If serum containing polyclonal antibodies to an epitope obtainable from an identifed agent and/or substance of the present invention contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against epitopes obtainable from an identifed agent and/or substance of the present invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against orbit epitopes can be screened for various properties; i.e., for isotype and epitope affinity.

Monoclonal antibodies to the substance and/or identified agent of the present invention may be prepared using any technique which provides for the production of antibody molecules by continuous cell lines in culture. These include, but are not limited to, the hybridoma technique originally described by Koehler and Milstein (1975 Nature 256:495-497), the human B-cell hybridoma technique (Kosbor et al (1983) Immunol Today 4:72; Cote et al (1983) Proc Natl Acad Sci 80:2026-2030) and the EBV-hybridoma technique (Cole et al (1985) Monoclonal Antibodies and Cancer Therapy, Alan R Liss Inc, pp 77-96). In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454). Alternatively, techniques described for the production of single chain antibodies (U.S. Pat. No. 4,946,779) can be adapted to produce the substance specific single chain antibodies.

Antibodies, both monoclonal and polyclonal, which are directed against epitopes obtainable from an identifed agent and/or substance of the present invention are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the substance and/or agent against which protection is desired. Techniques for raising anti-idiotype antibodies are known in the art. These anti-idiotype antibodies may also be useful in therapy.

Antibodies may also be produced by inducing in vivo production in the lymphocyte population or by screening recombinant immunoglobulin libraries or panels of highly specific binding reagents as disclosed in Orlandi et al (1989, Proc Natl Acad Sci 86: 3833-3837), and Winter G and Milstein C (1991; Nature 349:293-299).

Antibody fragments which contain specific binding sites for the substance may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse W D et al (1989) Science 256:1275-1281).

Chemical Synthesis Methods

Typically the agent of the present invention will be prepared by chemical synthesis techniques.

The agent of the present invention or variants, homologues, derivatives, fragments or mimetics thereof may be produced using chemical methods to synthesize the agent in whole or in part. For example, peptides can be synthesized by solid phase techniques, cleaved from the resin, and purified by preparative high performance liquid chromatography (e.g., Creighton (1983) Proteins Structures And Molecular Principles, W H Freeman and Co, New York N.Y.). The composition of the synthetic peptides may be confirmed by amino acid analysis or sequencing (e.g., the Edman degradation procedure; Creighton, supra).

Direct synthesis of the agent or variants, homologues, derivatives, fragments or mimetics thereof can be performed using various solid-phase techniques (Roberge J Y et al (1995) Science 269: 202-204) and automated synthesis may be achieved, for example, using the ABI 431 A Peptide Synthesizer (Perkin Elmer) in accordance with the instructions provided by the manufacturer. Additionally, the amino acid sequences comprising the agent or any part thereof, may be altered during direct synthesis and/or combined using chemical methods with a sequence from other subunits, or any part thereof, to produce a variant agent, such as, for example, a variant a T-type channel and/or a variant SK (preferably SK3) channel.

In an alternative embodiment of the invention, the coding sequence of the agent or variants, homologues, derivatives, fragments or mimetics thereof may be synthesized, in whole or in part, using chemical methods well known in the art (see Caruthers M H et al (1980) Nuc Acids Res Symp Ser 215-23, Horn T et al (1980) Nuc Acids Res Symp Ser 225-232).

Mimetic

As used herein, the term “mimetic” relates to any chemical which includes, but is not limited to, a peptide, polypeptide, antibody or other organic chemical which has the same qualitative activity or effect as a reference entity.

Derivative

The term “derivative” or “derivatised” as used herein includes chemical modification of an agent. Illustrative of such chemical modifications would be replacement of hydrogen by a halo group, an alkyl group, an acyl group or an amino group.

Chemical Modification

In one embodiment of the present invention, the agent may be a chemically modified agent. The chemical modification of an agent of the present invention may either enhance or reduce hydrogen bonding interaction, charge interaction, hydrophobic interaction, Van Der Waals interaction or dipole interaction between the agent and the target.

In one aspect, the identified agent may act as a model (for example, a template) for the development of other compounds.

Pharmaceutical Compositions

The present invention also provides a pharmaceutical composition comprising a therapeutically effective amount of the agent of the present invention and a pharmaceutically acceptable carrier, diluent or excipients (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, the agents of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

Pharmaceutical compositions of the present invention may be provided in controlled release form. This can be achieved by providing a pharmaceutically active agent in association with a substance that degrades under physiological conditions in a predetermined manner. Degradation may be enzymatic or may be pH-dependent.

Pharmaceutical compositions may be deigned to pass across the blood brain barrier (BBB). For example, a carrier such as a fatty acid, inositol or cholesterol may be selected that is able to penetrate the BBB. The carrier may be a substance that enters the brain through a specific transport system in brain endothelial cells, such as insulin-like growth factor I or II. The carrier may be coupled to the active agent or may contain/be in admixture with the active agent. Liposomes can be used to cross the BBB. WO-A-91/04014 describes a liposome delivery system in which an active agent can be encapsulated/embedded and in which molecules that are normally transported across the BBB (e.g. insulin or insulin-like growth factor I or II) are present on the liposome outer surface. Liposome delivery systems are also discussed in U.S. Pat. No. 4,704,355.

Different drug delivery systems may be used to administer pharmaceutical compositions of the present invention, depending upon the desired route of administration. Drug delivery systems are described, for example, by Langer (Science 249:1527-1533 (1991)) and by Illum and Davis (Current Opinions in Biotechnology 2: 254-259 (1991)).

In a preferred embodiment, the asents of the present invention are delivered systemically (such as orally, buccally, sublingually), more preferably orally.

Hence, preferably the agent is in a form that is suitable for oral delivery.

Administration

The term “administered” includes delivery by viral or non-viral techniques. Viral delivery mechanisms include but are not limited to adenoviral vectors, adeno-associated viral (AAV) vectos, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

The agents of the present invention may be administered alone but will generally be administered as a pharmaceutical composition—e.g. when the agent is in admixture with a suitable pharmaceutical excipient, diluent or carrier selected with regard to the intended route of administration and standard pharmaceutical practice.

For example, the agent can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, via the pensis, vaginal, epidural, sublingual.

It is to be understood that not all of the agent need be administered by the same route. Likewise, if the composition comprises more than one active component, then those components may be administered by different routes.

If the agent of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the agent; and/or by using infusion techniques.

For parenteral administration, the agent is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the agent of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the agent of the present invention can be administered in the form of a suppository or pessary, or it may be applied topically in the form of a gel, hydrogel, lotion, solution, cream, ointment or dusting powder. The agent of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compositions of the present invention may be administered by direct injection.

For some applications, preferably the agent is administered orally.

For some applications, preferably the agent is administered topically.

Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The agent and/or the pharmaceutical composition of the present invention may be administered in accordance with a regimen of from 1 to 10 times per day, such as once or twice per day.

For oral and parenteral administration to human patients, the daily dosage level of the agent may be in single or divided doses.

Depending upon the need, the agent may be administered at a dose of from 0.01 to 30 mg/kg body weight, such as from 0.1 to 10 mg/kg, more preferably from 0.1 to 1 mg/kg body weight. Naturally, the dosages mentioned herein are exemplary of the average case. There can, of course, be individual instances where higher or lower dosage ranges are merited.

Formulation

The agents of the present invention may be formulated into a pharmaceutical composition, such as by mixing with one or more of a suitable carrier, diluent or excipient, by using techniques that are known in the art.

Individual

As used herein, the term “individual” refers to vertebrates, particularly members of the mammalian species. The term includes but is not limited to domestic animals, sports animals, primates and humans.

Treatment

It is to be appreciated that all references herein to treatment include curative, palliative and prophylactic treatment.

Pharmaceutical Combinations

In general, the agent may be used in combination with one or more other pharmaceutically active agents. The other agent is sometimes referred to as being an auxiliary agent.

Bioavailability

Preferably, the compounds of the invention (and combinations) are orally bioavailable. Oral bioavailablity refers to the proportion of an orally administered drug that reaches the systemic circulation. The factors that determine oral bioavailability of a drug are dissolution, membrane permeability and metabolic stability. Typically, a screening cascade of firstly in vitro and then in vivo techniques is used to determine oral bioavailablity.

Dissolution, the solubilisation of the drug by the aqueous contents of the gastrointestinal tract (GIT), can be predicted from in vitro solubility experiments conducted at appropriate pH to mimic the GIT. Preferably the compounds of the invention have a minimum solubility of 50 mcg/ml. Solubility can be determined by standard procedures known in the art such as described in Adv. Drug Deliv. Rev. 23, 3-25, 1997.

Membrane permeability refers to the passage of the compound through the cells of the GIT. Lipophilicity is a key property in predicting this and is defined by in vitro Log D_{7.4} measurements using organic solvents and buffer. Preferably the compounds of the invention have a Log D_{7.4} of −2 to +4, more preferably −1 to +2. The log D can be determined by standard procedures known in the art such as described in J. Pharm. Pharmacol. 1990, 42:144.

Cell monolayer assays such as CaCO₂ add substantially to prediction of favourable membrane permeability in the presence of efflux transporters such as p-glycoprotein, so-called caco-2 flux. Preferably, compounds of the invention have a caco-2 flux of greater than 2×10⁻⁶ cms⁻¹, more preferably greater than 5×10⁻⁶ cms⁻¹. The caco flux value can be determined by standard procedures known in the art such as described in J. Pharm. Sci, 1990, 79, 595-600.

Metabolic stability addresses the ability of the GIT or the liver to metabolise compounds during the absorption process: the first pass effect. Assay systems such as microsomes, hepatocytes etc are predictive of metabolic liability. Preferably the compounds of the Examples show metabolic stablity in the assay system that is commensurate with an hepatic extraction of less then 0.5. Examples of assay systems and data manipulation are described in Curr. Opin. Drug Disc. Devel., 201, 4, 36-44, Drug Met. Disp., 2000, 28, 1518-1523.

Because of the interplay of the above processes further support that a drug will be orally bioavailable in humans can be gained by in vivo experiments in animals. Absolute bioavailability is determined in these studies by administering the compound separately or in mixtures by the oral route. For absolute determinations (% absorbed) the intravenous route is also employed. Examples of the assessment of oral bioavailability in animals can be found in Drug Met. Disp., 2001, 29, 82-87; J. Med Chem, 1997, 40, 827-829, Drug Met. Disp., 1999, 27, 221-226.

Diagnostic Kits

The present invention also includes a diagnostic composition or diagnostic methods or kits for (i) detection and measurement of a T-type channel and/or an SK (preferably SK3) channel activity and/or the coupling thereof in biological fluids and tissue; and/or (ii) localization of a T-type channel and/or an SK (preferably SK3) channel activity and/or the coupling thereof in tissues; and/or for (iii) the detection of a predisposition to a neurodegenarative condition, such as Parkinson's disease. In this respect, the composition or kit will comprise an entity that is capable of indicating the presence of one or more—or even the absence of one or more—targets, such as a T-type channel and/or an SK (preferably SK3) channel activity and/or the coupling thereof in a test sample. Preferably, the test sample is obtained from the Substantia nigra.

By way of example, the diagnostic composition may comprise any one of the nucleotide sequences mentioned herein or a variant, homologue, fragment or derivative thereof, or a sequence capable of hybridising to all or part of any one of the nucleotide sequence.

In one embodiment, the present invention provides a method, wherein said method utilises a nucleic acid probe or primer to determine whether or not said individual has a genetic defect affecting the structure and/or function of a T-type calcium channel and/or an SK (preferably SK3) potassium channel and/or the coupling thereof.

By way of example, the method may use a binding agent capable of binding to an epitope or other structural moiety in order to determine whether or not said individual has a defect affecting the structure of a T-type calcium channel and/or an SK (preferably SK3) potassium channel, or of a moiety operatively associated therewith.

The present invention also provides a diagnostic kit comprising a nucleic acid probe or primer or a binding agent.

The present invention also provides a kit comprising a detectable signal (e.g. a fluorescent label, a radioactive label) and/or means for providing a detectable change when the kit is in use (e.g. for providing an enzyme-catalysed change). The kit may comprise instructions for use in diagnosing a disorder in accordance with the present invention.

Probes

The diagnostic compositions and/or kits of the present invention may comprise probes such as nucleic acid hybridisation or PCR probes which are capable of detecting polynucleotide sequences, including genomic sequences, encoding a target coding region, such as an T-type channel and/or an SK (preferably SK3) channel coding region or closely related molecules, such as alleles. The specificity of the probe, i.e., whether it is derived from a highly conserved, conserved or non-conserved region or domain, and the stringency of the hybridisation or amplification (high, intermediate or low) will determine whether the probe identifies only naturally occurring target coding sequence, or related sequences. Probes for the detection of related nucleic acid sequences are selected from conserved or highly conserved nucleotide regions of target family members and such probes may be used in a pool of degenerate probes. For the detection of identical nucleic acid sequences, or where maximum specificity is desired, nucleic acid probes are selected from the non-conserved nucleotide regions or unique regions of the target polynucleotides. As used herein, the term “non-conserved nucleotide region” refers to a nucleotide region that is unique to a target coding sequence disclosed herein and does not occur in related family members.

PCR as described in U.S. Pat. No. 4,683,195, U.S. Pat. No. 4,800,195 and U.S. Pat. No. 4,965,188 provides additional uses for oligonucleotides based upon target sequences. Such oligomers are generally chemically synthesized, but they may be generated enzymatically or produced from a recombinant source. Oligomers generally comprise two nucleotide sequences, one with sense orientation (5′->3′) and one with antisense (3′<-5′) employed under optimised conditions for identification of a specific gene or condition. The same two oligomers, nested sets of oligomers, or even a degenerate pool of oligomers may be employed under less stringent conditions for detection and/or quantification of closely related DNA or RNA sequences.

The nucleic acid sequence for a target can also be used to generate hybridisation probes as previously described, for mapping the endogenous genomic sequence. The sequence may be mapped to a particular chromosome or to a specific region of the chromosome using well known techniques. These include in situ hybridisation to chromosomal spreads (Verma et al (1988) Human Chromosomes: A Manual of Basic Techniques, Pergamon Press, New York City), flow-sorted chromosomal preparations, or artificial chromosome constructions such as YACs, bacterial artificial chromosomes (BACs), bacterial PI constructions or single chromosome cDNA libraries.

In situ hybridisation of chromosomal preparations and physical mapping techniques such as linkage analysis using established chromosomal markers are invaluable in extending genetic maps. Examples of genetic maps can be found in Science (1995; 270:410f and 1994; 265:1981f). Often the placement of a gene on the chromosome of another mammalian species may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once a disease or syndrome has been crudely localised by genetic linkage to a particular genomic region any sequences mapping to that area may represent associated or regulatory genes for further investigation. The nucleotide sequence of the subject invention may also be used to detect differences in the chromosomal location due to translocation, inversion, etc. between normal, carrier or affected individuals.

Recombinant Methods

Typically the agent of the present invention is prepared by recombinant DNA techniques.

In one embodiment, preferably the target is a T-type channel and/or an SK (preferably SK3) channel activity and/or the coupling thereof.

For some aspects, such as the assay methods, preferably the T-type channel and/or an SK (preferably SK3) channel is prepared by recombinant DNA techniques.

Amino Acid Sequences

As used herein, the term “amino acid sequence” refers to peptide, polypeptide sequences, protein sequences or portions thereof.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

The amino acid sequence may be prepared isolated from a suitable source, or it may be made synthetically or it may be prepared by use of recombinant DNA techniques.

In one aspect, the present invention provides an amino acid sequence that is capable of acting as a target in an assay for the identification of one or more agents and/or derivatives thereof capable of affecting the amino acid sequence in order to modulate a T-type channel and/or an SK (preferably SK3) channel activity and/or the coupling thereof.

Preferably the target is a T-type channel and/or an SK (preferably SK3) channel activity and/or the coupling thereof.

Preferably, the T-type channel and/or an SK (preferably SK3) channel is isolated and/or purified.

The T-type channel and/or an SK (preferably SK3) channel may be in a substantially isolated form. It will be understood that the T-type channel and/or an SK (preferably SK3) channel may be mixed with carriers or diluents which will not interfere with the intended purpose of the channel and still be regarded as substantially isolated. The T-type channel and/or an SK (preferably SK3) channel may also be in a substantially purified form, in which case it will generally comprise the T-type channel and/or an SK (preferably SK3) channel in a preparation in which more than 90%, e.g. 95%, 98% or 99% of the preparation will be the channel.

Variants/Homologues/Derivatives of Amino Acid Sequences

It is possible to use known amino acid sequences for the T-type channel and/or an SK (preferably SK3) channel. However, the present invention also includes the use of homologous sequences obtained from any source and for example, synthetic peptides, as well as variants or derivatives thereof.

Thus, the present invention covers variants, homologues or derivatives of the amino acid sequences presented herein, as well as variants, homologues or derivatives of the nucleotide sequence coding for those amino acid sequences.

In the context of the present invention, a homologous sequence is taken to include an amino acid sequence which is at least 75, 85 or 90% identical, preferably at least 95 or 98% identical at the amino acid level over at least, for example, the known amino acid sequences. In particular, homology should typically be considered with respect to those regions of the sequence known to be essential for channel activity rather than non-essential neighbouring sequences. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” aligunent. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al 1984, Nucleic Acids Research 12:387). Examples of other software than can perform sequence comparisons include, but are not limited to, the BLAST package (see Ausubel et al 1999 ibid—Chapter 18), FASTA (Atschul et al 1990, J. Mol. Biol., 403-410) and the GENEWORKS suite of comparison tools. Both BLAST and FASTA are available for offline and online searching (see Ausubel et al 1999 ibid, pages 7-58 to 7-60). However it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nlm.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The terms “variant” or “derivative” in relation to the amino acid sequences of the present invention includes any substitution of, variation of, modification of, replacement of, deletion of or addition of one (or more) amino acids from or to the sequence providing the resultant amino acid sequence has a T-type channel and/or an SK (preferably SK3) channel activity, preferably having at least the same T-type channel and/or an SK (preferably SK3) channel activity as the known amino acid sequences.

By way of example, a known sequence may be modified for use in the present invention. Typically, modifications are made that maintain the binding specificity of the sequence. Amino acid substitutions may be made, for example from 1, 2 or 3 to 10 or 20 substitutions provided that the modified sequence retains the required channel activity. Amino acid substitutions may include the use of non-naturally occurring analogues.

The channel of the present invention may also have deletions, insertions or substitutions of amino acid residues which produce a silent change and result in a functionally equivalent channel. Deliberate amino acid substitutions may be made on the basis of similarity in polarity, charge, solubility, hydrophobicity, hydrophilicity, and/or the amphipathic nature of the residues as long as the activity of the channel is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Preferably, the isolated channel and/or purified channel and/or non-native channel is prepared by use of recombinant techniques.

Nucleotide Sequence

As used herein, the term “nucleotide sequence” is synonymous with the term “polynucleotide”.

The nucleotide sequence may be DNA or RNA of genomic or synthetic or of recombinant origin. The nucleotide sequence may be double-stranded or single-stranded whether representing the sense or antisense strand or combinations thereof.

For some applications, preferably, the nucleotide sequence is DNA.

For some applications, preferably, the nucleotide sequence is prepared by use of recombinant DNA techniques (e.g. recombinant DNA).

For some applications, preferably, the nucleotide sequence is cDNA.

For some applications, preferably, the nucleotide sequence may be the same as the naturally occurring form.

In one aspect, the present invention provides a nucleotide sequence encoding a substance capable of acting as a target in an assay (such as a yeast two hybrid assay) for the identification of one or more agents and/or derivatives thereof capable of modulating a T-type channel and/or an SK (preferably SK3) channel activity and/or the coupling thereof.

In one aspect of the present invention, the nucleotide sequence encodes a T-type channel and/or an SK (preferably SK3) channel.

It will be understood by a skilled person that numerous different nucleotide sequences can encode the same channel of the present invention as a result of the degeneracy of the genetic code. In addition, it is to be understood that skilled persons may, using routine techniques, make nucleotide substitutions that do not affect the channel encoded by the nucleotide sequence of the present invention to reflect the codon usage of any particular host organism in which the channel of the present invention is to be expressed. The terms “variant”, “homologue” or “derivative” in relation to the known nucleotide sequences include any substitution of, variation of, modification of, replacement of, deletion of or addition of one (or more) nucleic acid from or to the sequence providing the resultant nucleotide sequence encoding the channel has T-type channel and/or an SK (preferably SK3) channel activity, preferably having at least the same channel activity as the known sequences.

As indicated above, with respect to sequence homology, preferably there is at least 75%, more preferably at least 85%, more preferably at least 90% homology to the known sequences. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

The present invention also encompasses nucleotide sequences that are capable of hybridising selectively to the sequences presented herein, or any variant, fragment or derivative thereof, or to the complement of any of the above. Nucleotide sequences are preferably at least 15 nucleotides in length, more preferably at least 20, 30, 40 or 50 nucleotides in length.

Hybridisation

The term “hybridization” as used herein shall include “the process by which a strand of nucleic acid joins with a complementary strand through base pairing” as well as the process of amplification as carried out in polymerase chain reaction (PCR) technologies.

Nucleotide sequences of the invention capable of selectively hybridising to the nucleotide sequences presented herein, or to their complement, will be generally at least 75%, preferably at least 85 or 90% and more preferably at least 95% or 98% homologous to the corresponding complementary nucleotide sequence presented herein over a region of at least 20, preferably at least 25 or 30, for instance at least 40, 60 or 100 or more contiguous nucleotides. Preferred nucleotide sequences of the invention will comprise regions homologous to the nucleotide sequence set out in SEQ ID No 2 of the sequence listings of the present invention preferably at least 80 or 90% and more preferably at least 95% homologous to the nucleotide sequence set out in SEQ ID No 2 of the sequence listings of the present invention.

The term “selectively hybridizable” means that the nucleotide sequence, when used as a probe, is used under conditions where a target nucleotide sequence of the invention is found to hybridize to the probe at a level significantly above background. The background hybridization may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC {1×SSC=0.15 M NaCl, 0.015 M Na₃ Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Nucleotide sequences which are not 100% homologous to the sequences of the present invention but fall within the scope of the invention can be obtained in a number of ways. Other variants of the sequences described herein may be obtained for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the nucleotide sequence set out in SEQ ID No 2 of the sequence listings of the present invention under conditions of medium to high stringency. Similar considerations apply to obtaining species homologues and allelic variants of the amino acid and/or nucleotide sequences of the present invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences, such as the nucleotide sequence set out in SEQ ID No 2 of the sequence listings of the present invention. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the T-type channel and/or an SK (preferably SK3) channel encoded by the nucleotide sequences.

The nucleotide sequences of the present invention may be used to produce a primer, e.g. a PCR primer, a primer for an alternative amplification reaction, a probe e.g. labelled with a revealing label by conventional means using radioactive or non-radioactive labels, or the nucleotide sequences may be cloned into vectors. Such primers, probes and other fragments will be at least 15, preferably at least 20, for example at least 25, 30 or 40 nucleotides in length, and are also encompassed by the term nucleotide sequence of the invention as used herein.

The nucleotide sequences such as a DNA polynucleotides and probes according to the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Due to the inherent degeneracy of the genetic code, other DNA sequences which encode substantially the same or a functionally equivalent amino acid sequence, may be used to clone and express a T-type channel and/or an SK (preferably SK3) channel. As will be understood by those of skill in the art, for certain expression systems, it may be advantageous to produce the T-type channel and/or the SK (preferably SK3) channel—encoding nucleotide sequences possessing non-naturally occurring codons. Codons preferred by a particular prokaryotic or eukaryotic host (Murray E et al (1989) Nuc Acids Res 17:477-508) can be selected, for example, to increase the rate of the channel expression or to produce recombinant RNA transcripts having desirable properties, such as a longer half-life, than transcripts produced from naturally occurring sequence.

Vector

In one embodiment of the present invention, an agent of the present invention or an T-type channel and/or an SK (preferably SK3) channel may be administered directly to an individual.

In another embodiment of the present invention, a vector comprising a nucleotide sequence encoding an agent of the present invention or an T-type channel and/or an SK (preferably SK3) channel is administered to an individual.

Preferably the recombinant channel is prepared and/or delivered to a target site using a genetic vector.

As it is well known in the art, a vector is a tool that allows or faciliates the transfer of an entity from one environment to another. In accordance with the present invention, and by way of example, some vectors used in recombinant DNA techniques allow entities, such as a segment of DNA (such as a heterologous DNA segment, such as a heterologous cDNA segment), to be transferred into a host and/or a target cell for the purpose of replicating the vectors comprising the nucleotide sequences of the present invention and/or expressing the proteins of the invention encoded by the nucleotide sequences of the present invention. Examples of vectors used in recombinant DNA techniques include but are not limited to plasmids, chromosomes, artificial chromosomes or viruses.

The term “vector” includes expression vectors and/or transformation vectors.

The term “expression vector” means a construct capable of in vivo or in vitrolex vivo expression.

The term “transformation vector” means a construct capable of being transferred from one species to another.

“Naked DNA”

The vectors comprising nucleotide sequences encoding an agent of the present invention or a channel of the present invention for use in treating neurodegenerative disorders, such as Parkinson's disease may be administered directly as “a naked nucleic acid construct”, preferably further comprising flanking sequences homologous to the host cell genome.

As used herein, the term “naked DNA” refers to a plasmid comprising a nucleotide sequences encoding an agent of the present invention or a channel of the present invention together with a short promoter region to control its production. It is called “naked” DNA because the plasmids are not carried in any delivery vehicle. When such a DNA plasmid enters a host cell, such as a eukaryotic cell, the proteins it encodes (such as an agent of the present invention or a T-type channel and/or an SK (preferably SK3) channel) are transcribed and translated within the cell.

Non-Viral Delivery

Alternatively, the vectors comprising nucleotide sequences of the present invention or an agent of the present invention may be introduced into suitable host cells using a variety of non-viral techniques known in the art, such as transfection, transformation, electroporation and biolistic transformation.

As used herein, the term “transfection” refers to a process using a non-viral vector to deliver a gene to a target mammalian cell.

Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology 1996 14; 556), multivalent cations such as spermine, cationic lipids or polylysine, 1,2,-bis (oleoyloxy)-3-(trimethylammonio) propane (DOTAP)-cholesterol complexes (Wolff and Trubetskoy 1998 Nature Biotechnology 16: 421) and combinations thereof.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Viral Vectors

Alternatively, the vectors comprising an agent of the present invention or nucleotide sequences of the present invention may be introduced into suitable host cells using a variety of viral techniques which are known in the art, such as for example infection with recombinant viral vectors such as retroviruses, herpes simplex viruses and adenoviruses.

Preferably the vector is a recombinant viral vectors. Suitable recombinant viral vectors include but are not limited to adenovirus vectors, adeno-associated viral (AAV) vectors, herpes-virus vectors, a retroviral vector, lentiviral vectors, baculoviral vectors, pox viral vectors or parvovirus vectors (see Kestler et al 1999 Human Gene Ther 10(10):1619-32). In the case of viral vectors, delivery of the nucleotide sequence encoding the channel is mediated by viral infection of a target cell.

Targeted Vector

The term “targeted vector” refers to a vector whose ability to infect/transfect/transduce a cell or to be expressed in a host and/or target cell is restricted to certain cell types within the host organism, usually cells having a common or similar phenotype.

Replication Vectors

The nucleotide sequences encoding an agent of the present invention or the channel of the present invention may be incorporated into a recombinant replicable vector. The vector may be used to replicate the nucleotide sequence in a compatible host cell. Thus in one embodiment of the present invention, the invention provides a method of making the channel of the present invention by introducing a nucleotide sequence of the present invention into a replicable vector, introducing the vector into a compatible host cell, and growing the host cell under conditions which bring about replication of the vector. The vector may be recovered from the host cell.

Expression Vector

Preferably, an agent of the present invention or a nucleotide sequence of present invention which is inserted into a vector is operably linked to a control sequence that is capable of providing for the expression of the coding sequence, such as the coding sequence of the channel of the present invention by the host cell, i.e. the vector is an expression vector. An agent of the present invention or a channel produced by a host recombinant cell may be secreted or may be contained intracellularly depending on the sequence and/or the vector used. As will be understood by those of skill in the art, expression vectors containing an agent of the present invention or the channel coding sequences can be designed with signal sequences which direct secretion of the agent of the present invention or the channel coding sequences through a particular prokaryotic or eukaryotic cell membrane.

Expression in vitro

The vectors of the present invention may be transformed or transfected into a suitable host cell and/or a target cell as described below to provide for expression of an agent of the present invention or a channel of the present invention. This process may comprise culturing a host cell and/or target cell transformed with an expression vector under conditions to provide for expression by the vector of a coding sequence encoding an agent of the present invention or the channel and optionally recovering the expressed agent of the present invention or channel. The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. The expression of an agent of the present invention or an channel of the invention may be constitutive such that they are continually produced, or inducible, requiring a stimulus to initiate expression. In the case of inducible expression, production of an agent of the present invention or a channel can be initiated when required by, for example, addition of an inducer substance to the culture medium, for example dexamethasone or IPTG.

Fusion Proteins

The channel or an agent of the present invention may be expressed as a fusion protein to aid in extraction and purification and/or delivery of the agent of the present invention or the channel to an individual and/or to facilitate the development of a screen for agents capable of modulating channel activity. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the activity of the target.

The fusion protein may comprise an antigen or an antigenic determinant fused to the substance of the present invention. In this embodiment, the fusion protein may be a non-naturally occurring fusion protein comprising a substance which may act as an adjuvant in the sense of providing a generalised stimulation of the immune system. The antigen or antigenic determinant may be attached to either the amino or carboxy terminus of the substance.

In another embodiment of the invention, the amino acid sequence may be ligated to a heterologous sequence to encode a fusion protein. For example, for screening of peptide libraries for agents capable of affecting the substance activity, it may be useful to encode a chimeric substance expressing a heterologous epitope that is recognized by a commercially available antibody.

Host Cells

A wide variety of host cells can be employed for expression of the nucleotide sequences encoding the agent—such as an agent of the present invention or an channel of the present invention. These cells may be both prokaryotic and eukaryotic host cells. Suitable host cells include bacteria such as E. coli, yeast, filamentous fungi, insect cells, mammalian cells, typically immortalized, e.g., mouse, CHO, human and monkey cell lines and derivatives thereof.

Examples of suitable expression hosts within the scope of the present invention are fungi such as Aspergillus species (such as those described in EP-A-0184438 and EP-A-0284603) and Trichoderma species; bacteria such as Bacillus species (such as those described in EP-A-0134048 and EP-A-0253455), Streptomyces species and Pseudomonas species; and yeasts such as Kluyveromyces species (such as those described in EP-A-0096430 and EP-A-0301670) and Saccharomyces species. By way of example, typical expression hosts may be selected from Aspergillus niger, Aspergillus niger var. tubigenis, Aspergillus niger var. awamori, Aspergillus aculeatis, Aspergillus nidulans, Aspergillus orvzae, Trichoderma reesei, Bacillus subtilis, Bacillus licheniformis, Bacillus amyloliquefaciens, Kluyveromyces lactis and Saccharomyces cerevisiae.

The use of suitable host cells—such as yeast, fungal and plant host cells—may provide for post-translational modifications (e.g. myristoylation, glycosylation, truncation, lapidation and tyrosine, serine or threonine phosphorylation) as may be needed to confer optimal biological activity on recombinant expression products of the present invention.

Preferred host cells are able to process the expression products to produce an appropriate mature polypeptide. Examples of processing includes but is not limited to glycosylation, ubiquitination, disulfide bond formation and general post-translational modification.

Screens

The present invention encompasses a range of screening methods.

Screening can be performed in vitro. This may be done for example by using neurons or parts thereof. For example calcium or potassium ion channels or membranes comprising such channels may be used. The calcium and potassium channels are preferably channels of a dopaminergic neuron, more preferably of a Substantia nigra dopaminergic neuron. Desirably they are T-type calcium channels and SK (preferably SK3) potassium channels.

Alternatively, animal models or computer models may be used. Non-human animals (e.g. primates) already possessing the calcium or potassium channels described herein may be used. Alternatively, transgenic nonhuman animals (e.g. rodents) capable of expressing the calcium or potassium ion channels may be produced. Techniques for producing transgenic animals are well known and are described e.g. in U.S. Pat. No. 4,870,009 and U.S. Pat. No. 4,873,191. For example, a nucleic acid encoding a desired polypeptide may be microinjected into a pronucleus of a fertilised oocyte. The oocyte may then be allowed to develop in a pseudopregnant female foster animal. The animal resulting from development of the oocyte can be tested (e.g. with antibodies) to determine whether or not it expresses the polypeptide. Alternatively, it can be tested with a probe to determine if it has a transgene. A transgenic animal can be used as a founder animal, which may be bred from in order to produce further transgenic animals. Two transgenic animals may be crossed. For example, in some cases transgenic animals may be haploid for a given gene and it may be desired to try to provide a diploid offspring via crossing. A transgenic animal may be cloned, e.g. by using the procedures set out in WO-A-97/07668 and WO-A-97/07699 (see also Nature 385:810-813 (1997)). Thus a quiescent cell can be provided and combined with an oocyte from which the nucleus has been removed combined. This can be achieved using electrical discharges. The resultant cell can be allowed to develop in culture and can then be transferred to a pseudopregnant female.

Calcium and/or potassium efflux through ion channels of animals models can be assayed, as can changes in neuronal firing mode.

Computer models can also be used. They include models of the channels as described above, particularly models predicting the effect of agents on the opening or closing of said channels and/or of changes in firing mode. Computer models may also be used to identify binding sites for binding agents. Computer-generated models include computer generated images. The model may be two or three dimensional, although this is not essential. It may comprise a plurality of co-ordinates (e.g. it may be a crystallographic image, such as an X-ray cystallographic image). It may be arranged to be rotatable or otherwise movable to enable different views to be taken.

Test agents capable of modulating the channel activity of targets may be screened in assays which are well known in the art. Screening may be carried out, for example in vitro, in cell culture, and/or in vivo. Biological screening assays may be based on but not limited to channel activity-based response models, binding assays (which measure how well an agent modulates channel activity), and bacterial, yeast and animal cell lines. The assays can be automated for high capacity-high throughput screening (HTS) in which large numbers of compounds can be tested to identify compounds with the desired channel modulating activity (see, for example WO 84/03564). Once an agent capable of modulating the channel activity—such as by modulating the opening time probability of the channel—has been identified, further steps may be carried out either to select and/or to modify compounds and/or to modify existing compounds, to improve the channel activity modulation capability.

Specific screens may be based upon mode of action of dopaminergic neuron

By way of example, one method of the present invention comprises providing an agent and determining whether or not it is capable of causing a change in the firing mode of a dopaminergic neuron, or of preventing a change that would otherwise occur. For example, the agent may cause the dopaminergic neuron to enter bursting mode or may prevent it from leaving bursting mode.

By way of further example, a further method comprises providing an agent and determining whether or not it is capable of reducing calcium efflux through a calcium channel or of reducing potassium efflux through a potassium channel. This can be used to identify channel blockers.

The channel is preferably a channel of a dopaminergic neuron, more preferably of a Substantia nigra dopaminergic neuron. Desirably the channel is a T-type calcium channel or an SK (preferably SK3) potassium channel.

As indicated above, screening can be performed by using individual neurons or parts thereof. For example, suction may be used to attach a micropipette to a part of a membrane. Current through ion channels in the membrane can then be recorded. If desired, a patch of membrane may be removed with the micropipette and used for screening (e.g. for measuring calcium or potassium flux). The advantage of this technique is that the ionic composition either side of the membrane can be readily adjusted. Membrane patches comprising only a single ion channel or only a few ion channels can therefore be obtained and used in this manner.

By way of further example, screens may be based upon binding studies. Here, binding agents to calcium or potassium channels or to moieties operatively associated with them (e.g. moieties capable of causing opening or closing of channels) may be useful as channel activators or blockers. Binding studies are therefore useful in identifying agents that may then be subjected to further screening as discussed above. Such studies include providing a binding agent and determining whether or not it binds to an ion channel or to a moiety operatively associated with the channel. If desired, the binding agent may be labelled to aid in identification.

In addition to various screening methods of the present invention, an agent identified or identifiable by such a screening method (of whatever nature) is within the scope of the present invention. This may itself be useful as a therapeutic agent or may be used in a drug development program leading to the provision of a therapeutic agent.

Medical uses of such therapeutic agents are within the scope of the present invention as are the drug development programs themselves and pharmaceutical compositions comprising such agents. A drug development program may, for example, involve taking an agent identified or identifiable by a screening method of the present invention, optionally modifying it (e.g. modifying its structure and/or providing a novel composition comprising said moiety) and performing further studies (e.g. toxicity studies and/or studies on activity, structure or function). Trials may be performed on non-human animals and may eventually be performed on humans. Such trials will generally include determining the effect(s) of different dosage levels. Drug development programs may utilise computers to analyse moieties identified by screening (e.g. to predict structure and/or function, to identify possible agonists or antagonists, to search for other moieties that may have similar structures or functions, etc.). Computers set up to perform such analyses are within the scope of the present invention, as are drugs developed based upon such analyses and uses thereof.

As indicated, any one or more of appropriate targets—such as an amino acid sequence and/or nucleotide sequence—may be used for identifying an agent capable of modulating T-type channel activity and/or SK (preferably SK3) channel activity and/or the coupling thereof in any of a variety of drug screening techniques. The target employed in such a test may be free in solution, affixed to a solid support, borne on a cell surface, or located intracellularly. The abolition of target activity or the formation of binding complexes between the target and the agent being tested may be measured.

Techniques for drug screening may be based on the method described in Geysen, European Patent Application 84/03564, published on Sep. 13, 1984. In summary, large numbers of different small peptide test compounds are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in a drug screening techniques. Alternatively, non-neutralising antibodies can be used to capture the peptide and immobilise it on a solid support.

This invention also contemplates the use of competitive drug screening assays in which neutralising antibodies capable of binding a target specifically compete with a test compound for binding to a target.

Another technique for screening provides for high throughput screening (HTS) of agents having suitable binding affinity to the substances and is based upon the method described in detail in WO 84/03564.

It is expected that the assay methods of the present invention will be suitable for both small and large-scale screening of test compounds as well as in quantitative assays.

Thus, the present invention provides a method of identifying agents that selectively modulating channel activity in the Substantia nigra of an individual, the method comprising contacting a suitable target from (or obtainable from) the Substantia nigra of an individual and then measuring the channel activity and/or extent of dopamine release.

The present invention also relates to a method of identifying agents that modulate the channel activity the method comprising contacting a suitable target with the agent and then measuring the activity and/or levels of expression of the channel.

The present invention also relates to a method of identifying agents that selectively modulate the channel activity the method comprising contacting a suitable target with the agent and then measuring the activity and/or levels of expression of the channel.

Assay Methods

The diagnostic compositions and/or methods and/or kits may be used in the following techniques which include but are not limited to; competitive and non-competitive assays, radioimmunoassay, bioluminescence and chemiluminescence assays, fluorometric assays, sandwich assays, immunoradiometric assays, dot blots, enzyme linked assays including ELISA, microtiter plates, antibody coated strips or dipsticks for rapid monitoring of urine or blood, immunohistochemistry and immunocytochemistry. By way of example, an immunohistochemistry kit may also be used for localization of channel activity in Substantia nigra. This immunohistochemistry kit permits localization of a channel in tissue sections and cultured cells using both light and electron microscopy which may be used for both research and clinical purposes. Such information may be useful for diagnostic and possibly therapeutic purposes in the detection and/or prevention and/or treatment of a neurodegenerative disorders, such as Parkinoson's disease. For each kit the range, sensitivity, precision, reliability, specificity and reproducibility of the assay are established. Intraassay and interassay variation is established at 20%, 50% and 80% points on the standard curves of displacement or activity.

Diagnostic Testing

In order to provide a basis for the diagnosis of disease, normal or standard values from a target should be established. This may be accomplished by combining body fluids or cell extracts taken from normal subjects, either animal or human, with, for example, an antibody to a target under conditions suitable for complex formation which are well known in the art. The amount of standard complex formation may be quantified by comparing it to a dilution series of positive controls where a known amount of antibody is combined with known concentrations of a purified target. Then, standard values obtained from normal samples may be compared with values obtained from samples from subjects potentially affected by a neurodegenerative disorder. Deviation between standard and subject values establishes the presence of the disease state.

A target itself, or any part thereof, may provide the basis for a diagnostic and/or a prophylactic and/or therapeutic compound. For diagnostic purposes, target polynucleotide sequences may be used to detect and quantify gene expression in conditions, disorders or diseases in which neurodegenerative disorder may be implicated.

The target encoding polynucleotide sequence may be used for the diagnosis of a neurodegenerative disorder resulting from expression of the target. For example, polynucleotide sequences encoding a target may be used in hybridisation or PCR assays of tissues from biopsies or autopsies or biological fluids, to detect abnormalities in target expression. The form of such qualitative or quantitative methods may include Southern or northern analysis, dot blot or other membrane-based technologies; PCR technologies; dip stick, pin or chip technologies; and ELISA or other multiple sample formal technologies. All of these techniques are well known in the art and are in fact the basis of many commercially available diagnostic kits.

Such assays may be tailored to evaluate the efficacy of a particular therapeutic treatment regime and may be used in animal studies, in clinical trials, or in monitoring the treatment of an individual patient. In order to provide a basis for the diagnosis of disease, a normal or standard profile for target expression should be established. This is accomplished by combining body fluids or cell extracts taken from normal subjects, either animal or human, with the target or a portion thereof, under conditions suitable for hybridisation or amplification. Standard hybridisation may be quantified by comparing the values obtained for normal subjects with a dilution series of positive controls run in the same experiment where a known amount of purified target is used. Standard values obtained from normal samples may be compared with values obtained from samples from subjects potentially affected by a disorder or disease related to expression of the target coding sequence. Deviation between standard and subject values establishes the presence of the disease state. If disease is established, an existing therapeutic agent is administered, and treatment profile or values may be generated. Finally, the assay may be repeated on a regular basis to evaluate whether the values progress toward or return to the normal or standard pattern. Successive treatment profiles may be used to show the efficacy of treatment over a period of several days or several months.

Thus, in one aspect, the present invention relates to the use of a target polypeptide, or variant, homologue, fragment or derivative thereof, to produce anti-target antibodies which can, for example, be used diagnostically to detect and quantify target levels in neurodegenerative disorder states.

The present invention further provides diagnostic assays and kits for the detection of a target in cells and tissues comprising a purified target which may be used as a positive control, and anti-target antibodies. Such antibodies may be used in solution-based, membrane-based, or tissue-based technologies to detect any disease state or condition related to the expression of target protein or expression of deletions or a variant, homologue, fragment or derivative thereof.

The diagnostic compositions and/or kits comprising these entites may be used for a rapid, reliable, sensitive, and specific measurement and localization of appropriate channel activity in appropriate tissue extracts.

Thus, in addition to the medical uses discussed herein, the present invention can be used in diagnosis. For example, a binding agent as discussed above may be used to determine whether or not a patient has an epitope or other structure that is not present in a healthy individual but is present in an individual with a disorder as described herein. Alternatively, the binding agent may be used to identify an epitope or other structure that is not present in an individual with the disorder, but is present in a healthy individual. The epitope or other structure may, for example, be part of an SK (preferably SK3) or T-type channel, or may be part of a moiety that is operatively associated with the channel (e.g. it may be part of a moiety that causes channel opening or closing).

A nucleic acid molecule may be used in attempted hybridisation studies to determine whether or not a patient carries a genetic defect (e.g. a defective gene) that may give rise to one or more of the disorders discussed above. Preferably the genetic defect affects the structure and/or function of an SK (preferably SK3) channel, a T-type channel, or a moiety operatively associated therewith (e.g. a moiety that causes channel opening or closing). The nucleic acid may be provided as a probe or primer (e.g. in a diagnostic kit). It may be labelled. The probe or primer may be used for PCR-based amplification. It may also be used for reverse PCR. Hybridisation studies may be performed under stringent conditions. Preferably these are sufficiently stringent to distinguish between single nucleotide changes so as to identify point mutations. Hybridisation conditions are discussed in detail at pp 1.101-1.110 and 11.45-11.61 of Sambrook et al [Molecular Cloning, 2nd Edition, Cold Spring Harbor Laboratory Press (1989)]. One example of stringent hybridisation conditions involves using a pre-washing solution of 5×SSC, 0.5% SDS, 1.0 mM EDTA (pH 8.0) and attempting hybridisation overnight at 55° C. using 5×SSC. However, there are many other possibilities. Some of these are listed in Table 1 of WO-A-98/45435, for example. Hybridisation can be followed by washes of increasing stringency. Thus initial washes may be under conditions of low stringency, but these can be followed with higher stringency washes, up to the stringency of the conditions under which hybridisation was performed.

Reporters

A wide variety of reporters may be used in the assay methods (as well as screens) of the present invention with preferred reporters providing conveniently detectable signals (eg. by spectroscopy). By way of example, a reporter gene may encode an enzyme which catalyses a reaction which alters light absorption properties.

Examples of reporter molecules include but are not limited to β-galactosidase, invertase, green fluorescent protein, luciferase, chloramphenicol, acetyltransferase, β-glucuronidase, exo-glucanase and glucoamylase. Alternatively, radiolabelled or fluorescent tag-labelled nucleotides can be incorporated into nascent transcripts which are then identified when bound to oligonucleotide probes.

In one preferred embodiment, the production of the reporter molecule is measured by the enzymatic activity of the reporter gene product, such as β-galactosidase.

A variety of protocols for detecting and measuring the expression of the target, such as by using either polyclonal or monoclonal antibodies specific for the protein, are known in the art. Examples include enzyme-linked immunosorbent assay (ELISA), radioimmunoassay (RIA) and fluorescent activated cell sorting (FACS). A two-site, monoclonal-based immunoassay utilising monoclonal antibodies reactive to two non-interfering epitopes on the target is preferred, but a competitive binding assay may be employed. These and other assays are described, among other places, in Hampton R et al (1990, Serological Methods, A Laboratory Manual, APS Press, St Paul Minn.) and Maddox D E et al (1983, J Exp Med 15 8:1211).

A wide variety of labels and conjugation techniques are known by those skilled in the art and can be used in various nucleic and amino acid assays. Means for producing labelled hybridisation or PCR probes for detecting the target polynucleotide sequences include oligolabelling, nick translation, end-labelling or PCR amplification using a labelled nucleotide. Alternatively, the target coding sequence, or any portion of it, may be cloned into a vector for the production of an mRNA probe. Such vectors are known in the art, are commercially available, and may be used to synthesize RNA probes in vitro by addition of an appropriate RNA polymerase such as T7, T3 or SP6 and labelled nucleotides.

A number of companies such as Pharmacia Biotech (Piscataway, N.J.), Promega (Madison, Wis.), and US Biochemical Corp (Cleveland, Ohio) supply commercial kits and protocols for these procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241. Also, recombinant immunoglobulins may be produced as shown in U.S. Pat. No. 4,816,567.

Additional methods to quantify the expression of a particular molecule include radiolabeling (Melby P C et al 1993 J Immunol Methods 159:235-44) or biotinylating (Duplaa C et al 1993 Anal Biochem 229-36) nucleotides, coamplification of a control nucleic acid, and standard curves onto which the experimental results are interpolated. Quantification of multiple samples may be speeded up by running the assay in an ELISA format where the oligomer of interest is presented in various dilutions and a spectrophotometric or calorimetric response gives rapid quantification.

Although the presence/absence of marker gene expression suggests that the gene of interest is also present, its presence and expression should be confirmed. For example, if the nucleotide sequence is inserted within a marker gene sequence, recombinant cells containing the same may be identified by the absence of marker gene function. Alternatively, a marker gene can be placed in tandem with a target coding sequence under the control of a single promoter. Expression of the marker gene in response to induction or selection usually indicates expression of the target as well.

Alternatively, host cells which contain the coding sequence for the target and express the target coding regions may be identified by a variety of procedures known to those of skill in the art. These procedures include, but are not limited to, DNA-DNA or DNA-RNA hybridisation and protein bioassay or immunoassay techniques which include membrane-based, solution-based, or chip-based technologies for the detection and/or quantification of the nucleic acid or protein.

Animal Models

In vivo models may be used to investigate and/or design therapies or therapeutic agents to treat neurodegenerative disorders, such as Parkinson's disease. The models could be used to investigate the effect of various tools/lead compounds on a variety of parameters which indicate dopamine release.

The invention further provides transgenic nonhuman animals capable of expressing the nucleotide sequence encoding the channel of the present invention or a variant, homologue, derivative or fragment thereof and/or a transgenic nonhuman animal having one or more nucleotide sequence encoding the channel of the present invention or a variant, homologue, derivative or fragment thereof inactivated. Expression of such a nucleotide sequence is usually achieved by operably linking the nucleotide sequence to a promoter and optionally an enhancer, and microinjecting the construct into a zygote. See Hogan et al., “Manipulating the Mouse Embryo, A Laboratory Manual,” Cold Spring Harbor Laboratory. Inactivation of such a nucleotide sequence may be achieved by forming a transgene in which a cloned nucleotide sequence is inactivated by insertion of a positive selection marker. See Capecchi, Science 244, 1288-1292 (1989). The transgene is then introduced into an embryonic stem cell, where it undergoes homologous recombination with an endogenous variant gene. Mice and other rodents are preferred animals. Such animals provide screens and/or screening systems for identifying agents capable of modulating channel activity.

Substantially Pure/Substantially Isolated

The term “substantially pure form” is used to indicate that a given component is present at a high level. The component is desirably the predominant component present in a composition. Preferably it is present at a level of more than 30%, of more than 50%, of more than 75%, of more than 90%, or even of more than 95%, said level being determined on a dry weight/dry weight basis with respect to the total composition under consideration. At very high levels (e.g. at levels of more than 90%, of more than 95% or of more than 99%) the component may be regarded as being in “isolated form”. Biologically active substances of the present invention (including polypeptides, nucleic acid molecules, binding agents, moieties identified/identifiable via screening, etc.) may be provided in a form that is substantially free of one or more contaminants with which the substance might otherwise be associated. Thus for example they may be substantially free of one or more potentially contaminating polypeptides and/or nucleic acid molecules. They may be provided in a form that is substantially free of other cell components (e.g. of cell membranes, of cytoplasm, etc.). When a composition is substantially free of a given contaminant, the contaminant will be at a low level (e.g. at a level of less than 10%, less than 5%, or less than 1% on the dry weight/dry weight basis set out above)

Alleviated

This term indicates that a subject—such as a patient—will receive a beneficial effect from a given treatment. The term is not restricted to complete cures or preventions of diseases or disorders, but includes any beneficial effect in respect of reducing deleterious symptoms of a disease or the rate of progression of a disease.

### SUMMARY

We have found that preferential coupling of small-conductance, calcium-activated potassium (SK) channels to T-type calcium channels prevents bursting in dopaminergic midbrain neurons.

Thus, in the light of our work, it is now known that Ca influx into T-type channels specifically activates SK (preferably SK3) channels which in turn specifically stimulate K efflux which in turn keep neurons in pace maker mode.

Thus, by blocking any one of these steps/modes of action it should be possible to decrease or prevent the stimulation of K efflux which in turn would cause the neurons to enter the burst mode and, in doing so, cause an increase in dopamine release.

The increase in dopamine release could be utilised in a number of clinical/medical fields—such as in the therapy of schizophrenia and Parkinson's disease.

Thus, aspects of the present invention relate to:

Use of an agent in the manufacture of a medicament, wherein said agent is capable of causing a dopaminergic neuron to enter bursting mode and/or of preventing it from leaving bursting mode.

Use of an agent in the manufacture of a medicament, wherein said agent is capable of affecting (in particular causing a change in) the firing mode of a dopaminergic neuron that causes a beneficial change in dopamine release from said dopaminergic neuron.

Use of an agent in the manufacture of a medicament, wherein said agent is capable of affecting (in particular causing a change in) the firing mode of a dopaminergic neuron that causes a beneficial increase in dopamine release from said dopaminergic neuron.

Use of an agent in the manufacture of a medicament, wherein said agent is capable of causing a dopaminergic neuron to enter bursting mode and/or of preventing it from leaving bursting mode, wherein said agent modulates: a T-type channel and/or an SK (preferably SK3) channel and/or the coupling of a T-type channel with an SK (preferably SK3) channel.

Use of an agent in the manufacture of a medicament, wherein said agent is capable of affecting (in particular causing a change in) the firing mode of a dopaminergic neuron that causes a beneficial change in dopamine release from said dopaminergic neuron, wherein said agent modulates: a T-type channel and/or an SK (preferably SK3) channel and/or the coupling of a T-type channel with an SK (preferably SK3) channel.

Use of an agent in the manufacture of a medicament, wherein said agent is capable of affecting (in particular causing a change in) the firing mode of a dopaminergic neuron that causes a beneficial increase in dopamine release from said dopaminergic neuron, wherein said agent modulates: a T-type channel and/or an SK (preferably SK3) channel and/or the coupling of a T-type channel with an SK (preferably SK3) channel.

Use of an agent in the manufacture of a medicament, wherein said agent is capable of causing a dopaminergic neuron to enter bursting mode and/or of preventing it from leaving bursting mode, wherein said agent blocks: a T-type channel and/or an SK (preferably SK3) channel and/or the coupling of a T-type channel with an SK (preferably SK3) channel.

Use of an agent in the manufacture of a medicament, wherein said agent is capable of affecting (in particular causing a change in) the firing mode of a dopaminergic neuron that causes a beneficial change in dopamine release from said dopaminergic neuron, wherein said agent blocks: a T-type channel and/or an SK (preferably SK3) channel and/or the coupling of a T-type channel with an SK (preferably SK3) channel.

Use of an agent in the manufacture of a medicament, wherein said agent is capable of affecting (in particular causing a change in) the firing mode of a dopaminergic neuron that causes a beneficial increase in dopamine release from said dopaminergic neuron, wherein said agent blocks: a T-type channel and/or an SK (preferably SK3) channel and/or the coupling of a T-type channel with an SK (preferably SK3) channel.

A method of treatment comprising administering to a subject in need of same an agent, wherein said agent is capable of causing a dopaminergic neuron to enter bursting mode and/or of preventing it from leaving bursting mode.

A method of treatment comprising administering to a subject in need of same an agent, wherein said agent is capable of causing a change in the firing mode of a dopaminergic neuron that causes a beneficial change in dopamine release from said dopaminergic neuron.

A method of treatment comprising administering to a subject in need of same an agent, wherein said agent is capable of affecting (in particular causing a change in) the firing mode of a dopaminergic neuron that causes a beneficial increase in dopamine release from said dopaminergic neuron.

A method of treatment comprising administering to a subject in need of same an agent, wherein said agent is capable of causing a dopaminergic neuron to enter bursting mode and/or of preventing it from leaving bursting mode, wherein said agent modulates: a T-type channel and/or an SK (preferably SK3) channel and/or the coupling of a T-type channel with an SK (preferably SK3) channel.

A method of treatment comprising administering to a subject in need of same an agent, wherein said agent is capable of affecting (in particular causing a change in) the firing mode of a dopaminergic neuron that causes a beneficial change in dopamine release from said dopaminergic neuron, wherein said agent modulates: a T-type channel and/or an SK (preferably SK3) channel and/or the coupling of a T-type channel with an SK (preferably SK3) channel.

A method of treatment comprising administering to a subject in need of same an agent, wherein said agent is capable of affecting (in particular causing a change in) the firing mode of a dopaminergic neuron that causes a beneficial increase in dopamine release from said dopaminergic neuron, wherein said agent modulates: a T-type channel and/or an SK (preferably SK3) channel and/or the coupling of a T-type channel with an SK (preferably SK3) channel.

A method of treatment comprising administering to a subject in need of same an agent, wherein said agent is capable of causing a dopaminergic neuron to enter bursting mode and/or of preventing it from leaving bursting mode, wherein said agent blocks: a T-type channel and/or an SK (preferably SK3) channel and/or the coupling of a T-type channel with an SK (preferably SK3) channel.

A method of treatment comprising administering to a subject in need of same an agent, wherein said agent is capable of affecting (in particular causing a change in) the firing mode of a dopaminergic neuron that causes a beneficial change in dopamine release from said dopaminergic neuron, wherein said agent blocks: a T-type channel and/or an SK (preferably SK3) channel and/or the coupling of a T-type channel with an SK (preferably SK3) channel.

A method of treatment comprising administering to a subject in need of same an agent, wherein said agent is capable of affecting (in particular causing a change in) the firing mode of a dopaminergic neuron that causes a beneficial increase in dopamine release from said dopaminergic neuron, wherein said agent blocks: a T-type channel and/or an SK (preferably SK3) channel and/or the coupling of a T-type channel with an SK (preferably SK3) channel.

An assay method for identifying and/or improving the effect (such as by using the assay in a drug development program to improve efficacy) of an agent, wherein said agent is capable of causing a dopaminergic neuron to enter bursting mode and/or of preventing it from leaving bursting mode.

An assay method for identifying and/or improving the effect of an agent, wherein said agent is capable of affecting (in particular causing a change in) the firing mode of a dopaminergic neuron that causes a beneficial change in dopamine release from said dopaminergic neuron.

An assay method for identifying and/or improving the effect of an agent, wherein said agent is capable of affecting (in particular causing a change in) the firing mode of a dopaminergic neuron that causes a beneficial increase in dopamine release from said dopaminergic neuron.

An assay method for identifying and/or improving the effect of an agent, wherein said agent is capable of causing a dopaminergic neuron to enter bursting mode and/or of preventing it from leaving bursting mode, wherein said agent modulates: a T-type channel and/or an SK (preferably SK3) channel and/or the coupling of a T-type channel with an SK (preferably SK3) channel.

An assay method for identifying and/or improving the effect of an agent, wherein said agent is capable of affecting (in particular causing a change in) the firing mode of a dopaminergic neuron that causes a beneficial change in dopamine release from said dopaminergic neuron, wherein said agent modulates: a T-type channel and/or an SK (preferably SK3) channel and/or the coupling of a T-type channel with an SK (preferably SK3) channel.

An assay method for identifying and/or improving the effect of an agent, wherein said agent is capable of affecting (in particular causing a change in) the firing mode of a dopaminergic neuron that causes a beneficial increase in dopamine release from said dopaminergic neuron, wherein said agent modulates: a T-type channel and/or an SK (preferably SK3) channel and/or the coupling of a T-type channel with an SK (preferably SK3) channel.

An assay method for identifying and/or improving the effect of an agent, wherein said agent is capable of causing a dopaminergic neuron to enter bursting mode and/or of preventing it from leaving bursting mode, wherein said agent blocks: a T-type channel and/or an SK (preferably SK3) channel and/or the coupling of a T-type channel with an SK (preferably SK3) channel.

An assay method for identifying and/or improving the effect of an agent, wherein said agent is capable of affecting (in particular causing a change in) the firing mode of a dopaminergic neuron that causes a beneficial change in dopamine release from said dopaminergic neuron, wherein said agent blocks: a T-type channel and/or an SK (preferably SK3) channel and/or the coupling of a T-type channel with an SK (preferably SK3) channel.

An assay method for identifying and/or improving the effect of an agent, wherein said agent is capable of affecting (in particular causing a change in) the firing mode of a dopaminergic neuron that causes a beneficial increase in dopamine release from said dopaminergic neuron, wherein said agent blocks: a T-type channel and/or an SK (preferably SK3) channel and/or the coupling of a T-type channel with an SK (preferably SK3) channel.

An assay method for identifying and/or improving the effect (such as by using the assay in a drug development program to improve efficacy) of an agent, wherein said assay utilises: a T-type channel and/or an SK (preferably SK3) channel and/or the coupling of a T-type channel with an SK (preferably SK3) channel, wherein said agent is capable of causing a dopaminergic neuron to enter bursting mode and/or of preventing it from leaving bursting mode.

An assay method for identifying and/or improving the effect of an agent, wherein said assay utilises: a T-type channel and/or an SK (preferably SK3) channel and/or the coupling of a T-type channel with an SK (preferably SK3) channel, wherein said agent is capable of affecting (in particular causing a change in) the firing mode of a dopaminergic neuron that causes a beneficial change in dopamine release from said dopaminergic neuron.

An assay method for identifying and/or improving the effect of an agent, wherein said assay utilises: a T-type channel and/or an SK (preferably SK3) channel and/or the coupling of a T-type channel with an SK (preferably SK3) channel, wherein said agent is capable of affecting (in particular causing a change in) the firing mode of a dopaminergic neuron that causes a beneficial increase in dopamine release from said dopaminergic neuron.

An assay method for identifying and/or improving the effect of an agent, wherein said assay utilises: a T-type channel and/or an SK (preferably SK3) channel and/or the coupling of a T-type channel with an SK (preferably SK3) channel, wherein said agent is capable of causing a dopaminergic neuron to enter bursting mode and/or of preventing it from leaving bursting mode, wherein said agent modulates: a T-type channel and/or an SK (preferably SK3) channel and/or the coupling of a T-type channel with an SK (preferably SK3) channel.

An assay method for identifying and/or improving the effect of an agent, wherein said assay utilises: a T-type channel and/or an SK (preferably SK3) channel and/or the coupling of a T-type channel with an SK (preferably SK3) channel, wherein said agent is capable of affecting (in particular causing a change in) the firing mode of a dopaminergic neuron that causes a beneficial change in dopamine release from said dopaminergic neuron, wherein said agent modulates: a T-type channel and/or an SK (preferably SK3) channel and/or the coupling of a T-type channel with an SK (preferably SK3) channel.

An assay method for identifying and/or improving the effect of an agent, wherein said assay utilises: a T-type channel and/or an SK (preferably SK3) channel and/or the coupling of a T-type channel with an SK (preferably SK3) channel, wherein said agent is capable of affecting (in particular causing a change in) the firing mode of a dopaminergic neuron that causes a beneficial increase in dopamine release from said dopaminergic neuron, wherein said agent modulates: a T-type channel and/or an SK (preferably SK3) channel and/or the coupling of a T-type channel with an SK (preferably SK3) channel.

An assay method for identifying and/or improving the effect of an agent, wherein said assay utilises: a T-type channel and/or an SK (preferably SK3) channel and/or the coupling of a T-type channel with an SK (preferably SK3) channel, wherein said agent is capable of causing a dopaminergic neuron to enter bursting mode and/or of preventing it from leaving bursting mode, wherein said agent blocks: a T-type channel and/or an SK (preferably SK3) channel and/or the coupling of a T-type channel with an SK (preferably SK3) channel.

An assay method for identifying and/or improving the effect of an agent, wherein said assay utilises: a T-type channel and/or an SK (preferably SK3) channel and/or the coupling of a T-type channel with an SK (preferably SK3) channel, wherein said agent is capable of causing a change in the firing mode of a dopaminergic neuron that causes a beneficial change in dopamine release from said dopaminergic neuron, wherein said agent blocks: a T-type channel and/or an SK (preferably SK3) channel and/or the coupling of a T-type channel with an SK (preferably SK3) channel.

An assay method for identifying and/or improving the effect of an agent, wherein said assay utilises: a T-type channel and/or an SK (preferably SK3) channel and/or the coupling of a T-type channel with an SK (preferably SK3) channel, wherein said agent is capable of affecting (in particular causing a change in) the firing mode of a dopaminergic neuron that causes a beneficial increase in dopamine release from said dopaminergic neuron, wherein said agent blocks: a T-type channel and/or an SK (preferably SK3) channel and/or the coupling of a T-type channel with an SK (preferably SK3) channel.

As indicated earlier, the agent can be a mixture of suitable agents.

Highly preferred aspects of the present invention relate to:

Use of an agent in the manufacture of a medicament, wherein said agent is capable of causing a dopaminergic neuron to enter bursting mode and/or of preventing it from leaving bursting mode, wherein said agent modulates: a T-type channel and/or an SK3 channel and/or the coupling of a T-type channel with an SK3 channel.

Use of an agent in the manufacture of a medicament, wherein said agent is capable of affecting (in particular causing a change in) the firing mode of a dopaminergic neuron that causes a beneficial change in dopamine release from said dopaminergic neuron, wherein said agent modulates: a T-type channel and/or an SK3 channel and/or the coupling of a T-type channel with an SK3 channel.

Use of an agent in the manufacture of a medicament, wherein said agent is capable of affecting (in particular causing a change in) the firing mode of a dopaminergic neuron that causes a beneficial increase in dopamine release from said dopaminergic neuron, wherein said agent modulates: a T-type channel and/or an SK3 channel and/or the coupling of a T-type channel with an SK3 channel.

Use of an agent in the manufacture of a medicament, wherein said agent is capable of causing a dopaminergic neuron to enter bursting mode and/or of preventing it from leaving bursting mode, wherein said agent blocks: a T-type channel and/or an SK3 channel and/or the coupling of a T-type channel with an SK3 channel.

Use of an agent in the manufacture of a medicament, wherein said agent is capable of affecting (in particular causing a change in) the firing mode of a dopaminergic neuron that causes a beneficial change in dopamine release from said dopaminergic neuron, wherein said agent blocks: a T-type channel and/or an SK3 channel and/or the coupling of a T-type channel with an SK3 channel.

Use of an agent in the manufacture of a medicament, wherein said agent is capable of affecting (in particular causing a change in) the firing mode of a dopaminergic neuron that causes a beneficial increase in dopamine release from said dopaminergic neuron, wherein said agent blocks: a T-type channel and/or an SK3 channel and/or the coupling of a T-type channel with an SK3 channel.

A method of treatment comprising administering to a subject in need of same an agent, wherein said agent is capable of causing a dopaminergic neuron to enter bursting mode and/or of preventing it from leaving bursting mode.

A method of treatment comprising administering to a subject in need of same an agent, wherein said agent is capable of affecting (in particular causing a change in) the firing mode of a dopaminergic neuron that causes a beneficial change in dopamine release from said dopaminergic neuron.

A method of treatment comprising administering to a subject in need of same an agent, wherein said agent is capable of affecting (in particular causing a change in) the firing mode of a dopaminergic neuron that causes a beneficial increase in dopamine release from said dopaminergic neuron.

A method of treatment comprising administering to a subject in need of same an agent, wherein said agent is capable of causing a dopaminergic neuron to enter bursting mode and/or of preventing it from leaving bursting mode, wherein said agent modulates: a T-type channel and/or an SK3 channel and/or the coupling of a T-type channel with an SK3 channel.

A method of treatment comprising administering to a subject in need of same an agent, wherein said agent is capable of affecting (in particular causing a change in) the firing mode of a dopaminergic neuron that causes a beneficial change in dopamine release from said dopaminergic neuron, wherein said agent modulates: a T-type channel and/or an SK3 channel and/or the coupling of a T-type channel with an SK3 channel.

A method of treatment comprising administering to a subject in need of same an agent, wherein said agent is capable of affecting (in particular causing a change in) the firing mode of a dopaminergic neuron that causes a beneficial increase in dopamine release from said dopaminergic neuron, wherein said agent modulates: a T-type channel and/or an SK3 channel and/or the coupling of a T-type channel with an SK3 channel.

A method of treatment comprising administering to a subject in need of same an agent, wherein said agent is capable of causing a dopaminergic neuron to enter bursting mode and/or of preventing it from leaving bursting mode, wherein said agent blocks: a T-type channel and/or an SK3 channel and/or the coupling of a T-type channel with an SK3 channel.

A method of treatment comprising administering to a subject in need of same an agent, wherein said agent is capable of affecting (in particular causing a change in) the firing mode of a dopaminergic neuron that causes a beneficial change in dopamine release from said dopaminergic neuron, wherein said agent blocks: a T-type channel and/or an SK3 channel and/or the coupling of a T-type channel with an SK3 channel.

A method of treatment comprising administering to a subject in need of same an agent, wherein said agent is capable of affecting (in particular causing a change in) the firing mode of a dopaminergic neuron that causes a beneficial increase in dopamine release from said dopaminergic neuron, wherein said agent blocks: a T-type channel and/or an SK3 channel and/or the coupling of a T-type channel with an SK3 channel.

An assay method for identifying and/or improving the effect (such as by using the assay in a drug development program to improve efficacy) of an agent, wherein said agent is capable of causing a dopaminergic neuron to enter bursting mode and/or of preventing it from leaving bursting mode.

An assay method for identifying and/or improving the effect of an agent, wherein said agent is capable of affecting (in particular causing a change in) the firing mode of a dopaminergic neuron that causes a beneficial change in dopamine release from said dopaminergic neuron.

An assay method for identifying and/or improving the effect of an agent, wherein said agent is capable of affecting (in particular causing a change in) the firing mode of a dopaminergic neuron that causes a beneficial increase in dopamine release from said dopaminergic neuron.

An assay method for identifying and/or improving the effect of an agent, wherein said agent is capable of causing a dopaminergic neuron to enter bursting mode and/or of preventing it from leaving bursting mode, wherein said agent modulates: a T-type channel and/or an SK3 channel and/or the coupling of a T-type channel with an SK3 channel.

An assay method for identifying and/or improving the effect of an agent, wherein said agent is capable of affecting (in particular causing a change in) the firing mode of a dopaminergic neuron that causes a beneficial change in dopamine release from said dopaminergic neuron, wherein said agent modulates: a T-type channel and/or an SK3 channel and/or the coupling of a T-type channel with an SK3 channel.

An assay method for identifying and/or improving the effect of an agent, wherein said agent is capable of affecting (in particular causing a change in) the firing mode of a dopaminergic neuron that causes a beneficial increase in dopamine release from said dopaminergic neuron, wherein said agent modulates: a T-type channel and/or an SK3 channel and/or the coupling of a T-type channel with an SK3 channel.

An assay method for identifying and/or improving the effect of an agent, wherein said agent is capable of causing a dopaminergic neuron to enter bursting mode and/or of preventing it from leaving bursting mode, wherein said agent blocks: a T-type channel and/or an SK3 channel and/or the coupling of a T-type channel with an SK3 channel.

An assay method for identifying and/or improving the effect of an agent, wherein said agent is capable of affecting (in particular causing a change in) the firing mode of a dopaminergic neuron that causes a beneficial change in dopamine release from said dopaminergic neuron, wherein said agent blocks: a T-type channel and/or an SK3 channel and/or the coupling of a T-type channel with an SK3 channel.

An assay method for identifying and/or improving the effect of an agent, wherein said agent is capable of affecting (in particular causing a change in) the firing mode of a dopaminergic neuron that causes a beneficial increase in dopamine release from said dopaminergic neuron, wherein said agent blocks: a T-type channel and/or an SK3 channel and/or the coupling of a T-type channel with an SK3 channel.

An assay method for identifying and/or improving the effect (such as by using the assay in a drug development program to improve efficacy) of an agent, wherein said assay utilises: a T-type channel and/or an SK3 channel and/or the coupling of a T-type channel with an SK3 channel, wherein said agent is capable of causing a dopaminergic neuron to enter bursting mode and/or of preventing it from leaving bursting mode.

An assay method for identifying and/or improving the effect of an agent, wherein said assay utilises: a T-type channel and/or an SK3 channel and/or the coupling of a T-type channel with an SK3 channel, wherein said agent is capable of affecting (in particular causing a change in) the firing mode of a dopaminergic neuron that causes a beneficial change in dopamine release from said dopaminergic neuron.

An assay method for identifying and/or improving the effect of an agent, wherein said assay utilises: a T-type channel and/or an SK3 channel and/or the coupling of a T-type channel with an SK3 channel, wherein said agent is capable of affecting (in particular causing a change in) the firing mode of a dopaminergic neuron that causes a beneficial increase in dopamine release from said dopaminergic neuron.

An assay method for identifying and/or improving the effect of an agent, wherein said assay utilises: a T-type channel and/or an SK3 channel and/or the coupling of a T-type channel with an SK3 channel, wherein said agent is capable of causing a dopaminergic neuron to enter bursting mode and/or of preventing it from leaving bursting mode, wherein said agent modulates: a T-type channel and/or an SK3 channel and/or the coupling of a T-type channel with an SK3 channel.

An assay method for identifying and/or improving the effect of an agent, wherein said assay utilises: a T-type channel and/or an SK3 channel and/or the coupling of a T-type channel with an SK3 channel, wherein said agent is capable of affecting (in particular causing a change in) the firing mode of a dopaminergic neuron that causes a beneficial change in dopamine release from said dopaminergic neuron, wherein said agent modulates: a T-type channel and/or an SK3 channel and/or the coupling of a T-type channel with an SK3 channel.

An assay method for identifying and/or improving the effect of an agent, wherein said assay utilises: a T-type channel and/or an SK3 channel and/or the coupling of a T-type channel with an SK3 channel, wherein said agent is capable of affecting (in particular causing a change in) the firing mode of a dopaminergic neuron that causes a beneficial increase in dopamine release from said dopaminergic neuron, wherein said agent modulates: a T-type channel and/or an SK3 channel and/or the coupling of a T-type channel with an SK3 channel.

An assay method for identifying and/or improving the effect of an agent, wherein said assay utilises: a T-type channel and/or an SK3 channel and/or the coupling of a T-type channel with an SK3 channel, wherein said agent is capable of causing a dopaminergic neuron to enter bursting mode and/or of preventing it from leaving bursting mode, wherein said agent blocks: a T-type channel and/or an SK3 channel and/or the coupling of a T-type channel with an SK3 channel.

An assay method for identifying and/or improving the effect of an agent, wherein said assay utilises: a T-type channel and/or an SK3 channel and/or the coupling of a T-type channel with an SK3 channel, wherein said agent is capable of affecting (in particular causing a change in) the firing mode of a dopaminergic neuron that causes a beneficial change in dopamine release from said dopaminergic neuron, wherein said agent blocks: a T-type channel and/or an SK3 channel and/or the coupling of a T-type channel with an SK3 channel.

An assay method for identifying and/or improving the effect of an agent, wherein said assay utilises: a T-type channel and/or an SK3 channel and/or the coupling of a T-type channel with an SK3 channel, wherein said agent is capable of affecting (in particular causing a change in) the firing mode of a dopaminergic neuron that causes a beneficial increase in dopamine release from said dopaminergic neuron, wherein said agent blocks: a T-type channel and/or an SK3 channel and/or the coupling of a T-type channel with an SK3 channel.

As indicated earlier, the agent can be a mixture of suitable agents.

In a highly preferred aspect, the agent is for the treatment of Parkinson's disease.

### EXAMPLES

The invention will now be further described only by way of example in which reference is made to the following Figures:

### FIGURES

FIG. 1 which shows a series of graphs;

FIG. 2 which shows a series of graphs;

FIG. 3 which shows a series of graphs;

FIG. 4 which shows a series of graphs; and

FIG. 5 which shows a series of graphs.

In more detail:

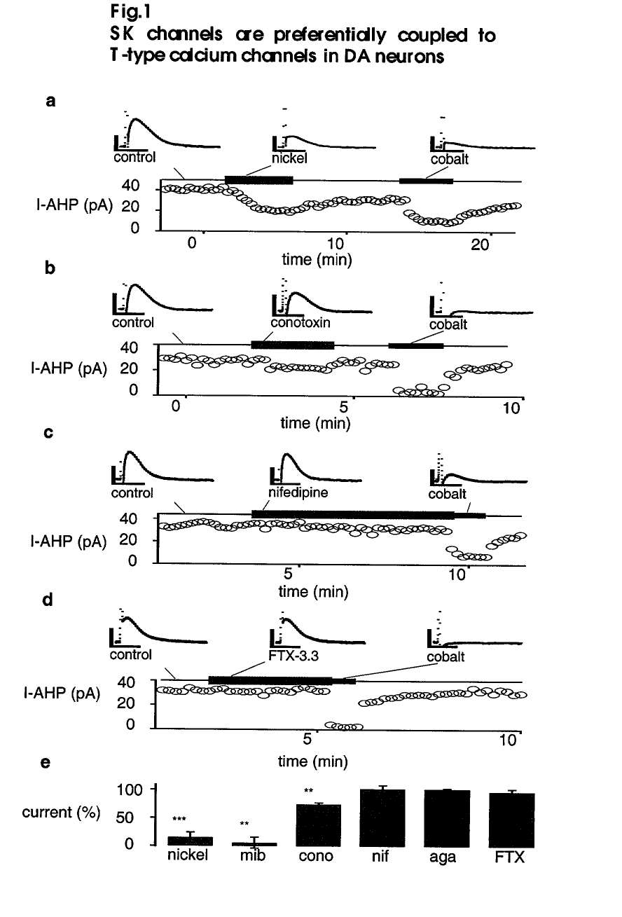

FIG. 1. Sensitivity of SK mediated AHP currents to inhibitors of voltage-sensitive calcium channels (T-type, L-type, N-type, P/Q-type). AHP currents (I-AHPs) were evoked by 20 ms hybrid clamp depolarizations using the perforated patch-clamp configuration (recording potential −60 mV). (a) Low micromolar concentrations (100 μM) of nickel (T-type) reversibly inhibited most of the cobalt-sensitive I-AHP. (b) Conotoxin GVIA (1 μM, N-type) reversibly reduced a minor part of the cobalt-sensitive I-AHP. (c) Nifedipine (10 μM, L-type) did not affect I-AHPs. (d) FTX-3.3 (1 μM, P/Q-type) had no effect on I-AHPs (see also agatoxin, e). (e) The summary of experiments as in (a-d) shows that hybrid-clamp evoked, cobalt-sensitive I-AHPs were preferentially activated via calcium channels sensitive to low micromolar (100 μM) nickel (85±9%, n=13) and mibefradil (94±10%, n=6), most likely T-type channels. A minor contribution of N-type channels was detected (1 μM conotoxin GVIA, 26±3%, n=4). L-type and P/Q-type did not significantly contribute to SK channel activation (10 μM nifedipine: residual current 102±6%, n=6; 1 μM FTX-3.3: residual current 97±5%, n=5; 0.1 μM agatoxin TK: residual current 101±1%, n=3). AHP current amplitudes were normalized to cobalt-sensitive component in each individual experiment exept for mibefradil were the mean value of cobalt block was used (1 mM, residual current 29±4%, n=29). Scale bars in (a-d) 0.3 s and 20 pA.

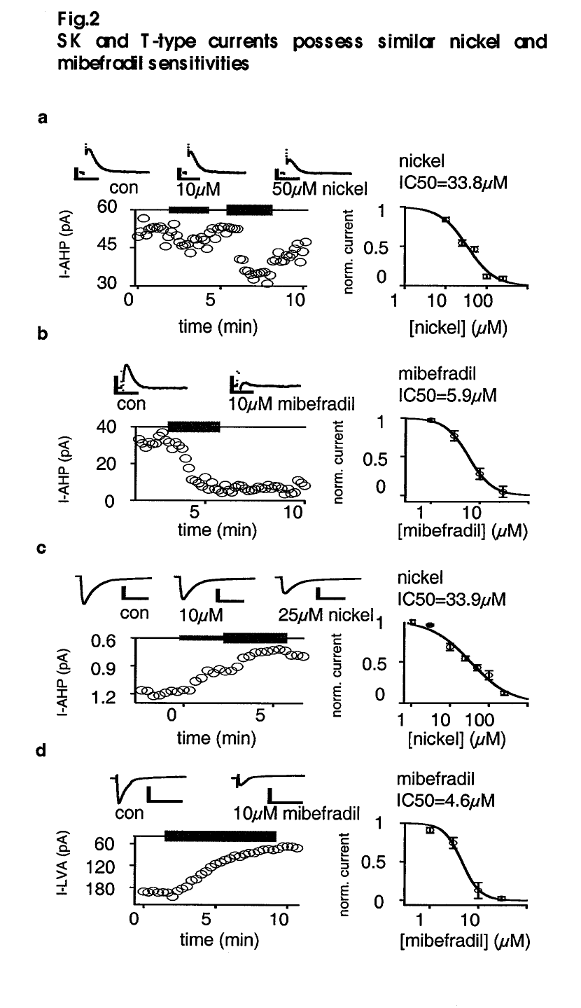

FIG. 2. SK mediated AHP currents and T-type channels possess very similar nickel and mibefradil sensitivities in dopaminergic neurons. (a) AHP currents (I-AHPs) evoked by 20 ms hybrid clamp depolarizations using the perforated patch-clamp configuration (recording potential −60 mV). Nickel reduced most of the cobalt-sensitive (see FIG. 1) I-AHP in a concentration dependent manner. The mean dose-response for nickel I-AHP inhibition was described by a Hill function with an IC₅₀ of 33.8 μM and a Hill coefficient of 1.3 (n=30). (b) Mibefradil inhibited a major component of the I-AHP irreversibly. The mean dose-response for mibefradil I-AHP inhibition was described by a Hill function with an IC₅₀ of 5.9 μM (Hill coef. 1.8, n=19). (c) Low voltage-activated calcium currents (I-LVA) evoked by depolarizations to −50 mV from a holding potential of −100 mV were recorded using standard whole-cell recordings (block of other currents see methods). Nickel reduced the I-LVA in a concentration dependent manner. The mean dose-response for nickel I-LVA inhibition was described by a Hill function with an IC₅₀ of 33.9 μM (Hill coef. 0.9, n=26). (b) Mibefradil inhibited the I-LVA irreversibly. The mean dose-response for mibefradil I-LVA inhibition was described by a Hill function with an IC₅₀ of 4.6 μM (Hill coef. 2.3, n=18). Scale bars 0.3 s, 20 pA in (a-b) and 0.3 s, 500 pA in (c-d).

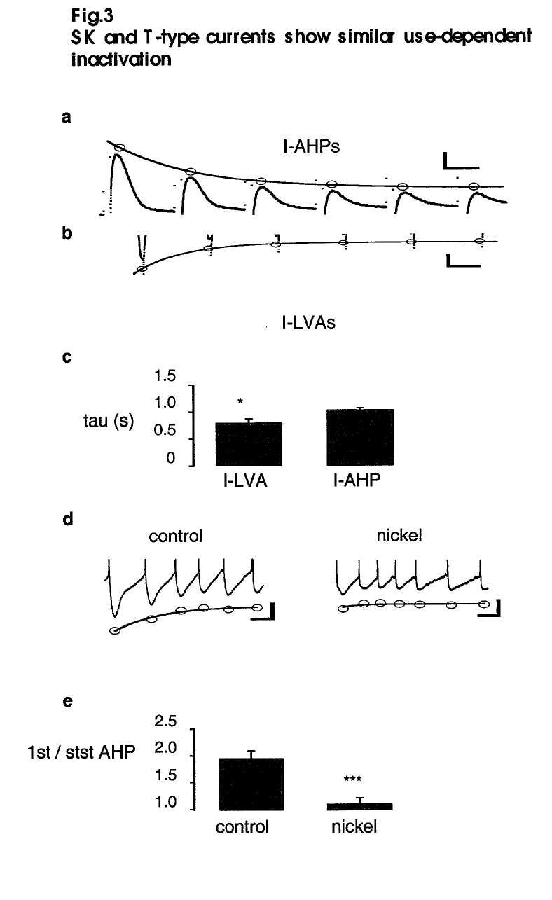

FIG. 3. Use-dependent inactivation of SK and T-type currents display similar kinetics. (a) AHP currents (I-AHP) evoked with hybrid-clamp depolarizations (100 ms, +60 mV) at a frequency of 1 Hz using the standard whole-cell configuration (recording potential −80 mV). Successive AHP currents decreased reaching a steady state level at 38% of the initial amplitude. The time constant of cumulative inactivation was 1.26 s. Scale bars 0.5 s and 50 pA. (b) Recording of low voltage-activated currents (I-LVA) evoked by the same voltage pulse protocol as in (a) using the standard whole-cell configuration and calcium channel recording solutions (see methods). Successive activation at 1 Hz lead to a decrease of I-LVAs reaching a steady state level of 42%. The time constant of use-dependent inactivation was 0.77 s. Scale bars 0.5 s and 200 pA. (c) Time constants (tau) of cumulative inactivation determined by experiments as in (a-b). Both, LVA and AHP currents had cumulative inactivation time constants in the range of one second: I-LVA, 0.80±0.07 s (n=10) and I-AHP, 1.04±0.03 s (n=61, p<0.05). (d) Perforated current-clamp recording of a train of action potentials evoked by injections of 10 pA for 4s from a hyperpolarized membrane potential of −80 mV. At the onset of depolarization AHPs were large and decreased with successive action potentials to a steady state (stst). Application of nickel (250 μM) decreased AHP amplitudes and the effect of cumulative inactivation. Note that the control rate of cumulative AHP inactivation (tau=1.10 s) was similar to time constants determined for I-AHPs and I-LVAs (see above). Scale bars 0.5 s and 10 mV. (e) The summary of experiments as in (d) shows that AHPs were reduced 2-fold under control conditions (2.0±0.1, n=8) whereas the effect was abolished by nickel application (1.1±0.1, n=8, p<0.0005).

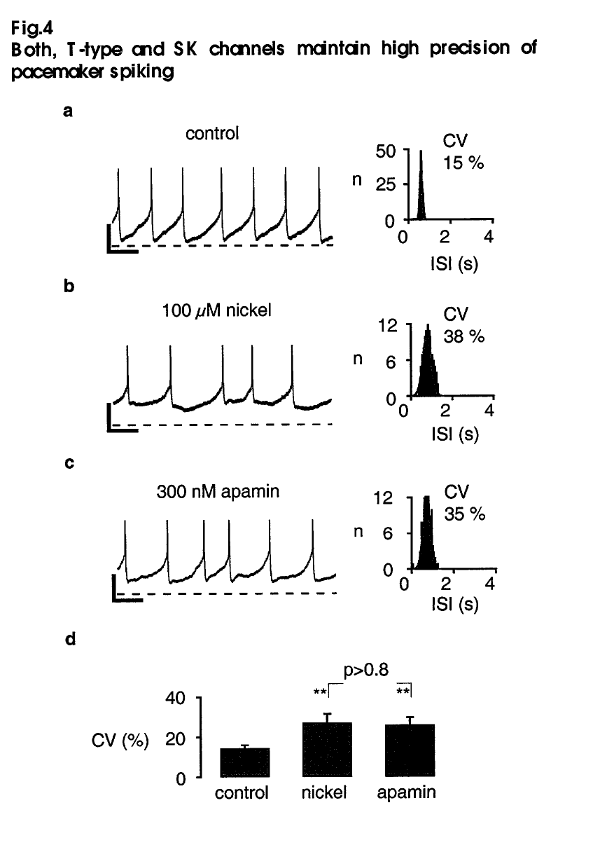

FIG. 4. Nickel-sensitive T-type and apamin-sensitive SK channels maintain the high precision of pacemaker spiking in dopaminergic neurons. (a-c) Perforated current-clamp recordings during control (a), 100 μM nickel (b) and 300 nM apamin (c) application. Left panels show a 4 s recording trace representative of 5 min recording for each condition. Interspike interval (ISI) frequency distributions are displayed in the right panels for each recording. As a measure of pacemaker precision the coefficient of variation (CV) was calculated from the gaussian fit of ISI histograms. Dotted line −50 mV. Scale bars 0.5 s and 10 mV. Action potentials are truncated at −20 mV. (a) During control conditions pacemaker spiking was very regular with a CV of 15%. (b) Application of 100 μM nickel reversibly rendered pacemaker spiking irregular (CV=38%). (c) Application of 300 nM apamin did increase the irregularity of pacemaker spiking to a similar degree (CV=35%). (d) The summary of experiments as in (a-c) shows that nickel (100 μM) and apamin (300 nM) decreased the pacemaker precision (p<0.005 respectively) to a similar extend (p>0.8). Mean CVs: control 14±2% (n=11), nickel 27±5% (n=11) and apamin 26±4% (n=11).

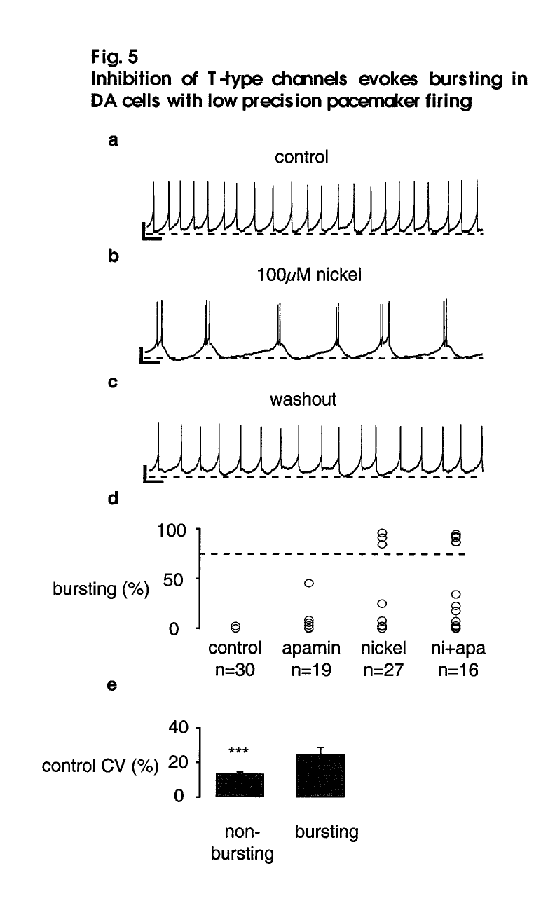

FIG. 5. Inhibition of T-type channels evokes bursting in a subpopulation of dopaminergic midbrain neurons. (a-c) Perforated current-clamp recordings during control (a), nickel (b) and washout (c) conditions. A 20 s recording trace representative of 5 min recording is shown for each condition. As a measure of pacemaker precision the coefficient of variation (CV) was calculated from gaussian fits of ISI histograms (not shown, see FIG. 4). Dotted line −50 mV. Scale bars 1 s and 10 mV. (a) This neuron showed pacemaker spiking at the lower end of firing precision (CV=20%, compare FIG. 4). (b) Application of 100 μM nickel switched the firing pattern from pacemaker to bursting with 2-3 closly spaced action potentials alternating with long inter burst intervals. (e) Upon washout of nickel the firing pattern returned to (irregular) pacemaker spiking. (d) Firing patterns during perforated patch-clamp recordings as in (a-b) and FIG. 4 were assessed by a burst detection program (see methods) that counted action potentials involved in bursts normalized to the total number of action potentials per recording (bursting in %, e.g. 85% bursting for the recording shown in c). Dotted line indicates 75% bursting. Under control conditions the bursting value was 0% (n=30). Application of 300 nM apamin did not significantly change the bursting value (3±2%, n=19) although one cell showed an increased value (45%) due to short periods of “burst-like” pattern. Inhibition of T-type channels with 100 μM nickel significantly increased the bursting value to 12±6% (n=27, p<0.05) and 3 cells displayed bursting values above 84%. The combination of nickel and apamin application (ni+apa) was most effective in switching from pacemaker to bursting behaviour increasing the mean bursting value to 34±10% (n=16, p<0.0005) with 5 neurons reaching bursting values of more than 86%. (e) Differential effects of T-type channel inhibition on firing patterns were asociated to pacamaker precision under control conditions (compare this figure and FIG. 4). Control CV values were correlated with the effect of nickel and apamin application on firing patterns of respective cells: neurons that were converted to bursting had significantly higher CV values (25+4%, n=6) compared to cells that became irregular upon nickel (or nickel+apamin) application (14±1%, n=22, p<0.0005).

Experimental Section A

Methods

Slice Preparation

Young (10-14d) C57B1/6J mice were killed by cervical dislocation in accordance with the Animals (Scientific Procedures) Act, 1986 (UK). Brains were immersed in ice-cold, artificial cerebrospinal fluid (ACSF) containing (in mM) 125 NaCl, 25 NaHCO₃, 2.5 KCl, 1.25 NaH₂PO₄, 2 CaCl₂, 2 MgCl₂ and 25 Glucose. Coronal brain slices (250 μm) were cut and held in ACSF bubbled with 95% O₂/5% CO₂ at room temperature (21-24° C.).

Electrophysiological Recordings

Neurons of the substantia nigra were visualized by infrared differential interference contrast video microscopy. Recordings were carried out at room temperature (except see below) using an EPC-9 patch-clamp amplifier and the software PULSE+PULSEFIT. To elicit and record AHP currents (I-AHPs) under voltage-clamp conditions short, unclamped (hybrid) depolarizations were used to evoke voltage-activated calcium influx. We have previously shown that I-AHPs of DA neurons are mainly mediated by apamin-sensitive SK currents using standard whole-cell recordings and 100 ms hybrid pulses to +60 mV from a holding/recording potential of −80 mV (used here only in FIG. 3a) (1).

In the present study, we aimed to simulate single action potentials and limit I-AHP rundown by using the perforated configuration (see below), 20 ms hybrid pulses and a recording/holding potential of −60 mV.

For current clamp and hybrid clamp recordings (except see above) the perforated whole-cell configuration was used (11). Patch pipettes were tip filled with a solution containg (in mM) 140 KMeSO₄, 5 KCl, 10 HEPES, 0.1 EGTA, 2 MgCl₂, pH 7.4. Amphotericin B (stock 100 mg/ml DMSO) was dilituted to 0.4 mg/ml in the same solution and backfilled into patch pipettes. Following gigaseal formation the perforation was monitored in the attached configuration untill a stable level of action potentials or I-AHPs was reached. Recorded cells were perfused locally with application solution containing (in mM) 145 NaCl, 2.5 KCl, 10 HEPES, 2 CaCl₂, 2 MgCl₂, 25 glucose, 0.05 picrotoxin, 0.05 kynurenic acid, pH 7.4. Current-clamp recordings were conducted either at room temperature or at 36-37° C. As there was no significant difference in spiking patterns data were pooled for the analysis shown in FIG. 5.

Low voltage-activated calcium channels were recorded in the standard whole-cell configuration and patch pipettes filled with a solution containing (in mM) 140 TEACl, 10 HEPES, 10 EGTA, 2 MgCl₂, pH 7.3. Recorded neurons were locally perfused with an application solution containing (in mM) 145 TEACl, 2.5 CsCl, 10 HEPES, 2 CaCl₂, 2 MgCl₂, 25 glucose, pH 7.4. Additionally, the control solution contained 50 μM picrotoxin, 5 μM kynurenic acid, 0.5 μM tetrodotoxin, 4 mM 4-AP, 10 μM nifedipine, 10 nM ω-conotoxin. Drugs were diluted in the respective application solutions and applied locally (<50 μm) at 50-100 μl/min under visual control.

Analysis

For analysis and plotting the software IgorPro was used. Shown voltage-clamp traces were filtered at 100 Hz and averaged from 3-10 traces at steady state levels. For spiking pattern analysis a burst detector was programmed in IgorPro. The burst detector compared all interspike intervals (ISIs) of a trace with its mean spiking rate and detected the coincidence of a short ISI (<0.5*mean rate) with a long ISI (>1.25*mean rate) within 2-7 consecutive spikes and marked it as a “burst”. Intervals within the burst were additionally tested by a poisson surprise mechanism that compared ISIs with the poisson distribution of all ISIs of a recording (12). The spikes within bursts were summed up and normalized to the total number of spikes in the trace. All values are mean±S.E.M. Significance values are p<0.05 (*).

Experimental Discussion

Introduction

Dopaminergic midbrain (DA) neurons are important for movement, cognition, reward and are implicated in disorders such as schizophrenia and Parkinson's disease. We have recently shown that SK3, a member of the small-conductance, calcium-activated potassium channel family, controls the frequency and precision of pacemaker spiking in DA neurons of the substantia nigra (SN), but not the ventral tegmental area (1). The aim of the present study was to determine the calcium source of SK channel activation in SN DA neurons of mice. SK channels are assumed to be activated via voltage-sensitive calcium channels (VSCCs) and in hippocampal neurons it may be specifically the L-type VSCC that activates SK channels (2). All major VSCC types (L-type, N-type, P/Q-type and T-type) have been show to be present in DA neurons and are therefore potential calcium sources for SK channel activation (3, 4). Alternatively (or as an amplifying mechanism) calcium release from internal stores may activate SK channels (5). We recorded SK-mediated afterhyperpolarization currents (I-AHP) while applying different VSCC blockers to determine the calcium source of SK channel activation using the perforated patch-clamp technique and hybrid clamp protocols.

Our results demonstrate that SK channels are preferentially activated by T-type calcium channels.

In vivo, DA SN neurons show two different spontaneous modes of action potential firing, single spike firing and burst firing of closely spaced action potentials separated by long pauses (6). However, DA neurons recorded in brain slices only display regular, pacemaker spiking. This, together with the ability of NMDA receptor activation to produce burst firing (7), is why it is generally assumed that burst firing is a synaptic mechanism (8). However, it has also been suggested that burst firing may be an intrinsic mechanism as the SK selective blocker apamin produced burst firing in some rat brain slice recordings of DA cells (9). In agreement with the latter hypothesis our present results suggest that blocking T-type channels, the major calcium source of SK channel activation (see above) can induce in vitro bursting. This indicates that, in contrast to thalamocortical neurons where T-type channels promote bursting (10), the role of T-type channels is reversed in DA neurons by their functional coupling to SK channels.

Summary

Our results suggest that SK channels are activated preferentially via T-type channels in mouse SN DA neurons:

1. Application of T-type channel blockers during perforated patch-clamp recording of hybrid pulse evoked AHP currents reduced 85-94% of the calcium-sensitive I-AHP, whereas other VSCC blockers either had no effect (L-type, P/Q-type) or blocked only minor components (26%, N-type).

2. Quantitative pharmacology of I-AHP and LVA currents shows that SK and T-type currents possess very similar sensitivities for mibefradil and nickel.

3. SK and T-type currents display a similar use-dependent inactivation that defines temporal AHP behaviour.

4. SK and T-type channels are equally important to maintain high precision of pacemaker spiking in DA neurons, as shown with perforated current clamp recordings.

Inhibition of T-type channels evokes bursting in a subpopulation of DA neurons. This indicates a bursting mechanism different from the one described in other vertebrate brain neurons. Our results suggest that T-type calcium and small-conductance, calcium-activated potassium (SK) channels constitute a functional signalling complex in mouse DA neurons which stabilizes pacemaker spiking and prevents burst firing.

Experimental Section B

Introduction

Dopaminergic (DA) midbrain neurones play an essential role in a variety of brain functions such as voluntary movement, working memory, and reward (Goldman-Rakic, 1999; Kitai et al., 1999; Spanagel and Weiss, 1999). In addition, they are intimately involved in neuropsychiatric and neurological disorders such as schizophrenia, drug addiction, and Parkinson's disease (Dunnett and Bjorklund, 1999; Verhoeff, 1999; Svensson, 2000). Since these brain functions and diseases are associated with anatomically distinct DA neurone subpopulations, it will be important to understand the function of distinct mesencephalic DA neurones under physiological and pathophysiological conditions. In vivo DA neurones exert their function by integrating synaptic inputs with their prominent intrinsic pacemaker activity to generate patterns of electrical activity that control dendritic and synaptic dopamine release. The intrinsic pacemaker of DA neurones, which is preserved in in vitro brain slice preparations, is likely to be orchestrated by a specific set of coexpressed ion channels. Previous studies have highlighted a number of ionic conductances that influence pacemaker activity, but the molecular identity of the respective ion channels was unknown. Also, most in vitro electrophysiological studies (Grace and Onn, 1989; Lacey et al., 1989; Yung et al., 1991; Richards et al., 1997) have considered DA midbrain neurones as a single population with homogeneous properties regardless of their anatomical diversity. In contrast, DA midbrain neurones respond in a heterogeneous, but topographically ordered fashion to the neurodegenerative process in Parkinson's disease and its animal models, where some DA subpopulations are highly vulnerable while others survive (German et al., 1996; Damier et al., 1999b; Betarbet et al., 2000). Although significant progress has been made by identification of mutant genes in familiar young-onset forms of PD (Hattori et al., 2000), it is still unknown how the well documented mitochondrial dysfunction initiates selective DA neurodegeneration (Kosel et al., 1999) and why there are dramatic differences in the survival rate of distinct DA subpopulations (Damier et al., 1999a). Thus, the research programme had two main targets. First, to define the molecular identities and functional roles of ion channels in DA pacemaker control on the background of the anatomical diversity of DA midbrain neurones. Secondly, to define, on a molecular level, pathophysiological mechanisms of neurodegeneration with respect to the differential vulnerability of DA midbrain neurones.

Molecular Physiology of Pacemaker Control in DA Midbrain Neurones

Ion channels that are active in the subthreshold range are good candidates for tuning the pacemaker frequency and precision in DA midbrain neurones (Grace, 1991; Amini et al., 1999; Kitai et al., 1999). The action potential in DA neurones initiates—at least in DA SN neurones—a prominent, calcium-dependent afterhyperpolarisation that is followed by a spontaneous slow pacemaker-depolarisation that leads the membrane potential back to threshold. As the first of these two phases, which constitute the interspike interval, is sensitive to calcium and apamin, we studied function as well as mRNA and protein expression of the small-conductance calcium-activated potassium (SK1-4) channel family (Bond et al., 1999). We defined the biophysical and quantitative pharmacological properties of native SK channels in DA neurones, which were consistent with our multiplex and semi-quantitative single-cell RT-PCR results and immunocytochemcial studies using SK antibodies. All sets of data agree that homomeric SK3 channels constitute the native apamin-sensitive K channels in DA SN neurones. Additional evidence came from our combined electrophysiological and semi-quantitative laser-confocal SK3 immunohistochemical experiments that showed a correlation between native SK current amplitudes and relative SK3 immunosignal intensities on the level of individual DA neurones. Furthermore, we demonstrated that SK3 channels control frequency (in a frequency-dependent manner) and precision of interspike intervals in DA SN neurones. Interestingly, a different, highly irregular discharging population of DA neurones in the VTA possessed a significantly lower density of SK3 channels and this small channel pool is not involved in pacemaker frequency control or stabilisation (Wolfart et al., 2001). The SK3-dependent afterhyperpolarisation is followed by an almost linear depolarisation back to the firing threshold. We found evidence that during this phase the inactivation of A-type potassium channels as well as the activity of hyperpolarisation-activated cyclic-nucleotide gated (Ih) channels both contribute to frequency control in DA SN neurones (Liss et al., 2001; Neuhoff et al., 2001). In DA SN neurones, which express a high density of Ih channels, selective Ih inhibition by 30 μM ZD7288 decreased the pacemaker frequency by about 50%. To understand the molecular composition of the Ih channel, we investigated the single-cell mRNA expression profiles of their candidate genes HCN1-4 in DA neurones (Franz et al., 2000). Using single-cell RT-multiplexPCR protocols, we showed that the slow-gating Ih phenotype in DA, and also in thalamocortical neurones, was correlated with the coexpression of HCN2, HCN3, and HCN4. In contrast, HCN1, which was expressed in hippocampal and cortical neurones with fast-gating Ih currents was consistently absent in DA SN neurones. To determine the functional stoichiometry of somato-dendritic Ih channels in DA neurones, further quantitative single-cell RT-PCR and immunocytochemical experiments are necessary.

Quantitative Real-Time Single-Cell RT-PCR: Linear Correlation Between Pacemaker Frequency and Ion Channel Transcript Number

The single-cell RT-PCR expression profiles for A-type K channel candidate genes were less complex. In DA SN neurones that displayed fast-inactivating A-type K currents with varying amplitudes but homogeneous biophysical gating properties, which was verified in nucleated outside-out patches, only the long splice variant of Kv4.3 of all candidate genes tested was expressed. The pharmacological and biophysical properties of native A-currents in DA SN neurones were consistent with the expression of Kv4 channels. The presence of Kv4.3 protein in TH-positive DA SN neurones was confirmed by immunohistochemistry. We observed that the variations of firing frequency within the physiological range of 0.5-5 Hz were highly correlated to variations of Kv4.3 channel density and selective inhibition of Kv4.3 channels by heteropodatoxin (HpTx3) increased the firing frequency. We developed a novel quantitative single-cell RT-PCR assay based on real-time fluorescence PCR (TaqMan primer/hybridisation probe assay) and demonstrated that the number of active Kv4.3 channels is not only tightly associated with the pacemaker frequency of individual DA SN neurones, but is also highly correlated with the number of Kv4.3L (long splice variant) mRNA molecules. Thus, the variation in Kv4.3L transcription is sufficient to explain the full spectrum of spontaneous pacemaker frequencies in identified DA SN neurones. This linear coupling between Kv4.3L mRNA abundance, A-type channel density and pacemaker frequency suggests a surprisingly simple molecular mechanism how DA SN neurones tune their variable firing rates by transcriptional control of a single ion channel gene (Liss et al., 2001)

The Functional Topography of DA Midbrain Neurones

The functional diversity of dopaminergic midbrain neurones both in SN and VTA was also studied. Combining electrophysiological analysis with cell labelling for morphological and neurochemical characterisation, we identified four DA midbrain subpopulations that possess significant differences in pacemaker activity and modes of subthreshold integration. The distribution of these DA neurone subpopulations is highly topographically organised and correlates with differential expression of the calcium-binding protein, calbindin D₂₈-K. We demonstrated that the large differences in Ih channel density are one key determinant for these DA subpopulations. It results in significant differences in temporal subthreshold integration and determines whether DA neurones increase or decrease their post-inhibitory excitability. Moreover, in only one of the DA subpopulations, the classical calbindin-negative SN neurones, are Ih channels directly involved in pacemaker control. We have also identified functionally relevant differences in the properties of other subthreshold channels—in particular A-type and SK channels—in these anatomically and neurochemically distinct DA subpopulations (Neuhoff et al., 2001; Wolfart et al., 2001). Thus, diversity within the DA system is not restricted to diverging axonal projections and differences in synaptic connectivity, but essentially involves differences between DA neurones in their somato-dendritic domains.

The Role of ATP-Sensitive Potassium (K-ATP) Channels in Differential Vulnerability of DA Midbrain Neurones to Neurodegeneration

We have identified that DA neurones in the substantia nigra (SN) respond differently to low nanomolar concentrations of the complex I inhibitor rotenone. One highly sensitive population of DA SN neurones hyperpolarized and completely lost its spontaneous activity in the presence of rotenone, while another population was not affected. We demonstrated that this differential response was mediated by the selective activation of K-ATP channels. Using single-cell RT-multiplex PCR, we showed that it was correlated to the differential mRNA expression of K-ATP subunits with SUR1/Kir6.2 expression being found in the rotenone-sensitive DA subpopulation (Liss et al., 1999b). Furthermore, in a genetic mouse model of dopaminergic neurodegeneration, the weaver mouse, we identified the gain-of-function cellular phenotype that was constituted by coactivation of mutant, non-selective weaverGIRK2 and K-ATP channels. The latter were partially compensating the weaverGIRK2-induced membrane depolarisation. In contrast to the wildtype mice, we detected only the highly metabolically sensitive SUR1/Kir6.2 K-ATP isoform in the surviving, calbindin-positive DA neurones in the weaver mouse (Liss et al., 1999a). Our results demonstrate for this mouse model of PD that differential expression and tonic functional activation of K-ATP channels play an active role in the cellular phenotype of surviving DA neurones and suggest that differential K-ATP channel expression is a molecular mechanism that contributes to the differential vulnerability of DA neurones. Recently, we studied the functional response to rotenone and also MPP⁺ in control and Kir6.2 knockout mouse. Consistent with our single-cell RT-PCR results, we demonstrated the absence of functional K-ATP channels in DA neurones of Kir6.2 knockout mouse. Moreover, the hyperpolarizing response to rotenone and MPP⁺ was completely lost in DA neurones of Kir6.2 knockout mouse, demonstrating that K-ATP channels constitute a selective and sensitive transducer of mitochondrial dysfunction in a subpopulation of DA neurones (Liss et al., 2000). Its sensitivity both for rotenone (EC₅₀≈15 nM) and MPP⁺ (EC₅₀≈2 μM) are in the range of concentrations, respectively that induces selective DA neurodegeneration in vivo. In this context, it is important to note that not only MPTP, but also a chronic challenge with nanomolar concentrations of rotenone was sufficient to induce selective degeneration of DA neurones (Betarbet et al., 2000). Currently, using a chronic MPTP mouse model of PD, we are investigating the question whether the loss of functional K-ATP channels and thus the membrane hyperpolarisation in response to neurotoxin-induced mitochondrial dysfunction in Kir6.2 knockout mice, affects the extent and pattern of DA neurodegeneration in vivo.

Hypothalamic K-ATP Channels are Essential for Glucose Homeostasis

Although our results provide evidence for a pathophysiological role of K-ATP channels in DA neurones, their physiological functions in neurones remain unclear. We observed in perforated patch-clamp recordings that the discharge rates of a subpopulation of DA neurones were controlled by physiological variations of the external glucose concentrations. We studied the potential role of K-ATP channels in a different brain region that is believed to form an important neural glucose sensor. We demonstrated that ventral-medial hypothalamic neurones like the metabolically sensitive DA subpopulation expressed SUR1/Kir6.2 mediated K-ATP channels that were absent in Kir6.2 KO mice. Moreover, these neurones had also lost their ability to alter their excitability in response to change in external glucose concentrations. In vivo experiments showed that this K-ATP channel-dependent glucose sensing in the hypothalamus was essential for glucagon release, glucose homeostasis and appetite control. Furthermore, this regulation was independent of leptin or NPY pathways controlling the food intake and energy balance (Miki et al., 2001). A specific contribution of glucose sensing in DA neurones needs to be further evaluated, but there is strong genetic evidence from transgenic mouse models that DA signalling is an essential stimulus for food intake (Zhou and Palmiter, 1995; Szczypka et al., 2000).

The Pathophysiology of Guanidinoacetate Methyltransferase (GAMT) Deficiency: the Accumulating Metabolite Guanidinoacetate Activates GABA_{A} Receptors

Human diseases that affect basal ganglia function are not necessarily disorders of advanced age. GAMT deficiency is a recently identified human disease of creatine biosynthesis that manifests postnatally with developmental delay and a chorea-like movement disorder. MRI imaging studies have highlighted selective lesions in the globus pallidus. Both the deficiency of high-energy phosphates in neurones and the neurotoxic action of the accumulating metabolite guanidinoacetate (GAA) are candidate mechanisms for the pathophysiology of this disease. In order to investigate the potential role of GAA accumulation in GAMT deficiency, we analysed the electrophysiological responses induced by GAA application on cultured neurones. GAA evoked GABA_{A} receptor mediated chloride currents with an EC₅₀ of 164 μM. In addition, clinically relevant concentrations of GAA hyperpolarized globus pallidus neurones in mouse brain slices and reduced their spike frequency. The GABA_{A} receptor antagonists picrotoxin and bicuculline blocked these GAA-induced effects. In contrast, millimolar concentrations of creatine had no effect on neuroneal activity. The GABA-mimetic action of GAA in brain and especially in the globus pallidus may be a candidate mechanism explaining the extrapyramidal dysfunction in patients with GAMT deficiency (Neu et al., 2000).

Summary

The aim of this aspect was to understand the genotype-phenotype correlation for single dopaminergic (DA) neurones combining functional electrophysiological with single-cell molecular-biological and immunohistochemical techniques. The two central topics are: a) the characterisation of differentially expressed ion channel genes, which determine the functional differences of DA neurone subpopulations; and b) the identification of differentially expressed ion channel genes involved in the selective vulnerability of degenerating DA neurones in Parkinson's disease.

We have developed and applied single-cell molecular techniques to analyse the mRNA expression profiles of ion channel candidate genes in combination with marker genes in single, functionally characterised DA neurones in mouse midbrain slices. Our protocols allow us a) to qualitatively assess the mRNA expression profiles of several candidate genes in parallel using RT-multiplexPCR techniques, b) to compare the relative abundance of detected mRNA species by serial dilution of single-cell cDNA pools, and c) to quantify the absolute transcript number of a selected target mRNA by quantitative real-time fluorescence RT-PCR. In addition, we have combined the functional characterisation in standard whole-cell and perforated-patch recordings with neurobiotin cell labelling, multi-labelling immunohistochemistry and confocal reconstruction to correlate the functional phenotype of individual DA neurone with its morphology, topographic position in the midbrain, and expression of channel and marker proteins. These studies together with investgations on mutant and knockout mouse models resulted in the following progress:

1. Identification of differential single-cell gene expression of K-ATP channel subunits in determining the metabolic sensitivity of DA neurones (Liss et al., 1999b)

2. Demonstration of the cellular phenotype and the selective expression and activation of SUR1-mediated K-ATP channels in DA neurones of the weaver mouse, a genetic model of Parkinson's disease (Liss et al., 1999a).

3. Identification of the role of differential HCN1 gene expression for the functional properties of neuronal Ih channels in DA neurones and other subcortical and cortical neurones (Franz et al., 2000; Neuhoff et al., 2001).

4. Discovery of the essential role of K-ATP channels in hypothalamic neurones for glucose homeostasis (Miki et al., 2001).

5. Definition of the molecular identity and functional role of calcium-activated SK channels in DA neurones (Wolfart et al., 2001).

6. Demonstration of the functional topography of DA midbrain neurones, where differential Ih channel expression is a key determinant for distinct functional phenotypes of DA neurone subpopulations (Neuhoff et al., 2000, 2001).

7. Development of quantitative real-time RT-PCR protocol on the level of individual neurones and its application to DA SN neurones (Liss et al., 2001).

8. Demonstration that guanidinoacetate (GA) acts via GABA_{A} receptor activation in GA methyltransferase deficiency, (Neu et al., 2000).

Understanding the molecular and cellular basis of electrical activity of DA neurones has important implications for major drug therapies targeting the dopaminergic system in particular in Parkinson's Disease and Schizophrenia. The identification of different DA subpopulations involved in distinct brain functions might be the first step to define novel, highly selective pharmacological approaches to affect selective dopaminergic subsystems. The high incidence of side effects of standard drug regimes in both Parkinson's disease and schizophrenia best illustrates the need for more selective treatment strategies. Understanding the molecular and cellular biology of selective vulnerability of DA neurones might contribute to novel neuroprotective strategies. The definition of distinct functional phenotypes of highly vulnerable and resistant DA neurones facilitates a transcriptome-wide search for differentially expressed genes that control their different fates in Parkinson's disease.

Experimental Section C

The aim of this aspect was to further understand the genotype-phenotype correlation of single dopaminergic (DA) neurones belonging to anatomically and functionally distinct subpopulations in the midbrain. The central aims are: a) the identification and functional characterisation of differentially expressed ion channels and channel-function modulating genes, which determine the discrete functional phenotypes of DA subpopulations; and b) the identification and functional validation of differentially expressed genes responsible for the differential vulnerability of DA neurones in Parkinson's disease and its mouse models.

In particular this aspect addresses the following issues:

I. Quantitative molecular physiology of DA midbrain neurone. What are the key ion channels underlying DA cell functional behaviour in pacemaker generation, frequency control, synaptic input and integration?

II. Molecular anatomy of DA midbrain neurones. What are the molecular and functional differences between DA neuronal populations innervating different brain areas? What is the molecular programme that determines differential axonal targeting of DA neurones?

III. Functional and molecular plasticity of DA midbrain neurones in mouse models of Parkinson's disease. What are the key molecular and functional changes in DA neurones in disease states in particular models of Parkinson's Disease?

IV. Functional genomics of DA neurones in Parkinson's disease. Which differentially expressed genes define the large differences of vulnerability towards neurodegeneration in DA neurones?

Tissues Investigated

The patch-clamp electrophysiology in combination with single-cell molecular-biological and immunohistochemical techniques on living DA neurones are carried out in acute midbrain slices and midbrain slice cultures of wildtype and transgenic mice. Perfused-fixed brains from control, treated and transgenic mice are used for immunohistochemical analysis of protein expression and DA neurone survival in disease models. In addition, laser-based microdissection of single cells or specific cell pools are carried out on fixed brain sections from mouse and post-mortem human brain, the latter from controls and Parkinson's disease patients.

Techniques

Patch-clamp electrophysiology in combination with qualitative and quantitative single-cell real-time RT-PCR and immunohistochemical light and confocal microscopic techniques. Single-cell electroporation for GFP expression labelling in vivo and ribozyme-based gene suppression in brain acute brain slices and slice cultures are used. In addition, antisense (a) RNA amplification and DNA-array based expression profiling are carried out. Laser-based microdissection are used to collect single DA neurones or specifically pre-labelled DA populations from fixed human or mouse brain section. In vivo stereotactic techniques are used in mice for retrograde labelling with fluorescent beads and alternative methods based on GFP expression vectors are evaluated. Finally, a chronic MPTP/probenicid-based mouse model of PD is used and the colony of K-ATP (Kir6.2) knockout mice maintained.

Measurements

Cell-attached, standard and perforated-whole cell patch-clamp recordings are used to study the electrical behaviour (e.g. spontaneous, synaptically evoked, during drug challenge, in disease models) of defined DA neurones, which may in some experiments be fluorescent-bead or GFP-labelled. Cell-attached, standard and perforated-whole cell as well as cell-free patch-clamp recordings are carried out to define the biophysical (permeability, selectivity, gating kinetics, current density, single channel profiles) and pharmacological properties of ion channels and signal transduction cascades of DA neurones. Qualitative mRNA expression profiles of relevant candidate genes are determined by single-cell RT-multiplex PCR. Quantitative expression profiling and genotype-phenotype correlations are carried out by single-cell real-time fluorescent RT-PCR (TaqMan primer/hybridisation probe assays). Localisation, morphology and the neurochemical phenotype of neurones and co-expression and co-localisation of ion channel and other target proteins are analysed by immunohistochemical techniques on the light and confocal microscopic level. Patch-clamp recordings are carried out before and after single-cell electroporation in ribozyme-based gene suppression experiments. Transcriptome-wide single-cell mRNA expression profiles of individual neurones are carried out by aRNA amplification and DNA-array hybridisation. Retrogradely labelled neurones are identified by fluorescence microscopy. Pattern and extent of DA neurodegeneration are determined by standard immunohistochemical and stereological techniques.

Anticipated Implications for Health Care

It is expected that the single-cell based genome-wide expression profiling (see IV) may identify genes involved in PD that constitute novel targets for developing neuroprotective therapies. The results may contribute to a better understanding of the molecular physiology of distinct DA subpopulations involved in PD, schizophrenia and drug abuse and could lead to more selective therapeutic approaches that could minimise the side effects that currently limit drug therapies in PD and schizophrenia.

Introduction

Dopaminergic (DA) midbrain neurones play an essential role in a variety of brain functions such as voluntary movement, working memory, and reward (Goldman-Rakic, 1999; Kitai et al., 1999; Spanagel and Weiss, 1999). In addition, they are intimately involved in neuropsychiatric and neurological disorders such as schizophrenia, drug addiction, and Parkinson's disease (Dunnett and Bjorklund, 1999; Verhoeff, 1999; Svensson, 2000). Since these brain functions and diseases are associated with anatomically distinct DA neurone subpopulations, the question arises whether the functional characteristics of mesencephalic DA neurones are differentiated in accordance to their anatomical subgroups. Within parallel mesostriatal (Maurin et al., 1999), mesolimbic (Groenewegen et al., 1999), and mesocortical networks (Tzschentke, 2001), DA neurones exert their function by integrating synaptic inputs in the context of their intrinsic pacemaker to generate pattern of electrical activity that control dopamine release and their functional effects on their respective target cells. Thus, potential diversity within the DA system might originate both from differences in axonal projections and synaptic connectivity as well as from diverging properties of the somato-dendritic integrator and pacemaker.

Anatomy of Dopaminergic Midbrain Neurones

Dopaminergic midbrain neurones are distributed in three partially overlapping nuclei: the retrorubral area (RRA, A8), substantia nigra (SN, A9), and the ventral tegmental area (VTA, A10), which correspond to different mesotelencephalic projections (Gardner and Ashby, 2000; Joel and Weiner, 2000). Substantia nigra neurones mainly target the dorsal striatum (mesostriatal projection) and are involved in motor function, whereas the neurones of the VTA project predominantly to the ventral striatum e.g. nucleus accumbens (mesolimbic projection) and to prefrontal cortex (mesocortical projection) and are thus associated with limbic functions (Gardner and Ashby, 2000; Joel and Weiner, 2000). Recent tracing studies have revealed a more refined concept of the topographical organisation of mesotelencephalic connections. In the SN, two neurochemically distinct tiers project to and receive input from different neurochemical compartments in the striatum, namely the limbic and sensori-motor regions (Maurin et al., 1999; Haber et al., 2000). Ventral tier DA neurones that do not express the calcium-binding protein calbindin D₂₈-K (calbindin-negative, CB−), project to patch compartments and in turn receive innervation from striatal projection neurones in the matrix. Conversely, calbindin-positive (CB+) dorsal tier DA neurones project to the matrix while receiving input from the limbic patch compartment (Gerfen, 1992; Barrot et al., 2000). Several studies have revealed that the striking differences in the vulnerability of DA neurones to degeneration in both Parkinson's disease and its animal models, are tightly associated with the differential expression of CB. Thus CB+DA neurones are less vulnerable (Liang et al., 1996; Damier et al., 1999a; Gonzalez-Hernandez and Rodriguez, 2000; Tan et al., 2000).

Physiology of Dopaminergic Neurones

In contrast to the well described anatomical and neurochemical organisation much less is known about the electrophysiology within different subpopulations of DA neurones. To date, in vitro electrophysiological studies have considered DA midbrain neurones mainly as a single population (Pucak and Grace, 1994; Kitai et al., 1999). These “classical” DA neurones have a number of features suggestive of the expression of a highly conserved repertoire of ion channels namely, low-frequency pacemaker activity, broad action potentials followed by a pronounced afterhyperpolarisation, a strong sag-component mediated by hyperpolarisation activated channels (Ih channels) and a D2 autoreceptor-mediated hyperpolarisation (Sanghera et al., 1984; Grace and Onn, 1989; Lacey et al., 1989; Richards et al., 1997). However, in vivo studies have highlighted significant differences in discharge rates, burst firing, and autoreceptor control between subgroups of DA midbrain neurones (Chiodo et al., 1984; Greenhoff et al., 1988; Shepard and German, 1988; Paladini et al., 1999). It is not known whether the observed diversity results from the differences in synaptic input, or relates to the differential expression of postsynaptic ion channels that control synaptic integration.

Functional Genomics of Differential Vulnerability of DA Neurones

Research carried out in the previous programme has contributed to our understanding of the diversity of DA neurones. We have identified electrophysiologically distinct DA phenotypes, which are topographically and neurochemically organised thus forming a functional landscape of discrete DA subpopulations (Neuhoff et al., 2000, 2001). In addition, a parallel DA map of equal importance needs to be considered, namely the well described topography of differential vulnerability and subpopulation-selective degeneration of DA neurones in Parkinson's Disease (Damier et al., 1999b) and its animal models (German et al., 1996; Betarbet et al., 2000). Similar to the anatomically organised distribution of different functional DA phenotypes, the pattern of selective vulnerability and degeneration has a defined topography. As we demonstrated, DA neurone subpopulations with large differences in vulnerability to neurodegeneration—for instance comparing calbindin-positive dorsal tier with calbindin-negative ventral tier DA neurones—also possess distinct functional phenotypes (Neuhoff et al., 2000, 2001).

This aspect of the present invention interconnects these distinct anatomical, physiological and pathophysiological maps of DA midbrain neurones on the level of individual neurones. It aims to define the molecular basis and to understand the functional implications of the observed diversity of DA neurones with respect to their electrophysiological function, anatomical connections, and vulnerability to neurodegeneration. Our combination of techniques on the single-cell level is of great advantage as it creates a compendium of single cell genotype-phenotype correlations. Post-genomic biology aimes to define molecular modules that correspond to a particular function as a cluster of co-expressed and co-regulated genes, whose products are likely to physically interact and localise to similar cellular compartments. This aspect addresses the molecular and functional definition of three important modules in DA neurones:

1. the pacemaker generation, intrinsic frequency control, and synaptic integration

2. the selective axonal targeting, branching and connectivity with target cells

3. the differential vulnerability to neurodegeneration in PD and its animal models

It combines two complementary strategies: A “single-gene-single-protein” approach based on previous experimental observations and non-biased large-scale expression profiling to fully capture the molecular diversity of DA neurones.

I. Quantitative Molecular Physiology of DA Midbrain Neurones

1 Pacemaker Frequency Control

Neuronal function is intimately coupled to cell-specific patterns of somatodendritic integration and generation of electrical activity. This cellular excitability is orchestrated in turn by a specific set of coexpressed ion channels. To date, in vitro electrophysiological studies have considered DA midbrain neurones mainly as a single population (Sanghera et al., 1984; Grace and Onn, 1989; Lacey et al., 1989; Richards et al., 1997). We have defined the molecular basis of important components of this cellular pacemaker and shown that SK3, HCN2-4, and Kv4.3L mediated ion channels are involved in controlling its frequency and precision (Franz et al., 2000; Liss et al., 2001; Neuhoff et al., 2001; Wolfart et al., 2001). Importantly, we have also identified functionally relevant differences in densities (Ih, IA, SK) and/or gating properties (IA) of subthreshold channels in anatomically and neurochemically distinct DA subpopulations (Neuhoff et al., 2000, 2001). Patch-clamp techniques with multiplex and quantitative single-cell RT-PCR (Liss et al., 1999b; Liss et al., 2001) and quantitative confocal immunohistochemistry (Wolfart et al., 2001) are used to address the following issues:

Molecular Basis of Differential Ih Channel Density in DA Neurones:

Quantitative single-cell analysis of HCN2, HCN3, HCN4 transcripts and immunocytochemical analysis are carried out with suitable antibodies. These experiments define, which of the coexpressed HCN2-4 subunits are relevant for somato-dendritic Ih channels in DA neurones and whether on a transcriptional level one or more HCN subunits correlate with the large differences in Ih current densities in DA subpopulations that we have identified. Single-cell electroporation (Haas et al., 2001) and ribozyme-based gene suppression techniques (Goodchild, 2000; Liu et al., 2000) for functional validation of the defined HCN genes are used. Ih currents are compared before and after HCN subtype-specific gene suppression in the same neurone. For comparison, heterologous expression of recombinant HCN channels are usefu. The analysis of emerging HCN transgenic or knockout mice may be integrated.

Molecular Basis of Differential A-Type Potassium (Kv4) Channel Gating in DA Neurones:

We have identified a DA subpopulation in the VTA that expresses A-type potassium channels that display five-fold slower inactivation kinetics in whole-cell and outside-out patches compared to those in “classical” DA SN neurones (Neuhoff et al., 2001). Recent studies on recombinant A-channels suggest an important role for Kv4 beta subunits (KChiPs) as gating modifiers of somato-dendritic Kv4 channels (An et al., 2000). Comparative multiplex and quantitative mRNA single-cell expression profiling of the KChiP gene family are carried in the different DA subpopulations. Immunocytochemical analyses are carried out with suitable antibodies. Single-cell electroporation (Haas et al., 2001) and ribozyme-based gene suppression techniques (Goodchild, 2000; Liu et al., 2000) for functional validation of the defined Kv4 and KChiP genes are used. A-type potassium currents are compared before and after Kv4/KChip subtype-specific gene suppression in the same neurone. For comparison, heterologous expression of heteromeric Kv4+ KChiP channels are used.

Metabolic Pacemaker Frequency Control by ATP-Sensitive Potassium (K-ATP) Channels in DA Neurones:

Using a combination of patch-clamp electrophysiology and single-cell RT-PCR and a Kir6.2 knockout mice (Seino et al., 2000) we demonstrated the molecular identity of neuronal K-ATP channels and their essential function in glucose sensing (Miki et al., 2001). The absence of K-ATP channels in DA neurones of Kir6.2 KO mice (Liss et al., 2000) confirmed our previous single-cell RT-PCR study (Liss et al., 1999b). We now have experimental evidence from cell-attached and perforated patch-clamp recordings that the pacemaker frequency of a subpopulation of DA neurones is controlled by physiological variation in the extracellular glucose concentrations. This is of particular interest, as some DA neurones are essential in feeding (Zhou and Palmiter, 1995; Szczypka et al., 2000; Wang et al., 2001) and might directly control cortical microcirculation (Krimer et al., 1998). A combination of perforated-patch clamp recordings to define glucose sensitivity with subsequent cell labelling and immunocytochemistry (Neuhoff et al., 2000; Wolfart et al., 2001) to define the topography of glucose-sensing DA neurones are used. mRNA expression profiling is used to define the set of differentially expressed genes including K-ATP channel subunits that render DA subpopulation glucose-sensitive. The functional role of a presumably mitochondrial K-ATP channel is studied in Kir6.1 knockout mice.

Pacemaker Generation

There is increasing evidence that a calcium-dependent signalling complex is the principal generator of spontaneous oscillatory potentials (SOP) that are necessary for pacemaker discharge (Amini et al., 1999; Wilson and Callaway, 2000). Previous studies in DA neurones implicated both nifedipine- and nickel-sensitive calcium channels in SOP generation (Yung et al., 1991; Nedergaard et al., 1993). Spontaneous calcium-activated potassium (SK) channel-mediated outward currents that are triggered by spontaneous calcium release from intracellular stores have also been described (Seutin et al., 2000). In addition, there is evidence for the presence of Gq-coupled or store-operated channels in DA neurones (Guatteo et al., 1999). However, the concerted interaction of voltage-gated calcium channels and calcium-activated potassium (SK, BK) channels with intracellular calcium pools and their respective calcium release (IP3-receptors, ryanodine receptors) and store-operated channels in the plasma membrane (TRPC1-7) (Harteneck et al., 2000; Strubing et al., 2001) remains to be defined. Furthermore, the molecular identities of the essential players of this signalling complex in DA neurones need to be determined. We propose to study function, expression and localisation of voltage-gated calcium channels, their coupling to BK and SK channels, and the role and molecular identify of intracellular calcium-release channels and store-operated channels. A combination of perforated patch-clamp studies to analyse DA neurones under conditions of intact calcium handling with biophysical and pharmacological studies on voltage-gated calcium channels and store-operated channels is used. As we have established a correlation between distinct functional phenotypes and differential calbindin expression (Neuhoff et al., 2000, 2001), the diversity of DA neurones will again have to be taken into account. On the molecular level, multiplex and quantitative single-cell RT-PCR techniques (Liss et al., 2001) and immunocytochemistry (Wolfart et al., 2001) are applied to define expression and localisation of the components of this signalling complex. Application of confocal calcium imaging techniques is evaluated to see how they could contribute to a better understanding of the spatial and temporal aspects of the calcium signalling complex that forms the generator of spontaneous activity in DA neurones (Wilson and Callaway, 2000).

Functional, Pharmacological and Molecular Characterisation of Voltage-Gated Calcium Channels in DA Neurones:

Perforated- and standard whole-cell as well as nucleated out-side out patch-clamp recordings are carried out to define the biophysical and pharmacological properties of voltage-gated calcium channels (VGCC) in DA neurones (Kang and Kitai, 1993b; Cardozo and Bean, 1995). To associate these functional profiles within distinct DA subpopulations they are also coupled with cell filling and immunocytochemistry. Qualitative multiplex and quantitative single-cell RT-PCR analysis may define the mRNA expression profile/s of (initially) important postsynaptic alpha-subunits (alpha1A-I) in respective DA subpopulation (Catterall, 1998; Lacinova et al., 2000). On the protein level guided by the mRNA expression profiling results, calcium channel recordings is combined with cell-filling and semi-quantitative analysis of immunostaining by alpha-subunit selective antibodies. Selective pharmacological inhibition of specific VGCCs are carried out in perforated-patch current-clamp recordings understand their specific contribution to pacemaker activity in distinct DA subpopulations. As alpha1D is a good candidate for nifedipine-sensitive VGCCs that generate SOPs in DA neurones (Takada et al., 2001), our analysis can be extended to study the DA pacemaker in alpha1D-knockout mice (Platzer et al., 2000).

Electrophysiological Coupling Studies Between Calcium Sources (Channels and Intracellular Pools) and Calcium-Sensors (SK and BK Channels):

In addition to their direct generator role in rhythmogenesis, VGCCs also provide the calcium influx that controls the activity of calcium-sensitive potassium channels like SK and BK (Marrion and Tavalin, 1998). For SK3 channels, we have recently shown that this mechanism is important for frequency and precision control of the DA pacemaker (Wolfart et al., 2001). To assess the functional contribution of large-conductance, calcium- and voltage-activated potassium (BK) channels for pacemaker regulation, a highly selective BK peptide blocker (iberiotoxin) is applied in perforated-patch current-clamp recordings of DA neurones. If interesting functional differences between DA populations are discovered, further mRNA expression studies are initiated. The hybrid-clamp method in perforated patch-clamp recordings to pharmacologically define those VGCCs that provide the calcium source for SK channels are used. Preliminary results suggest highly selective coupling between nickel-sensitive T-type VGCC (alpha1G-I) and SK3 channels, but no major role for L-type, N-type or P/Q-type VGCC channels (Wolfart and Roeper, 2001). These results suggest a high degree of functional specialisation and possibly spatial organisation of VGCC channels in DA neurones. In addition, SK activation might be further boosted by calcium release from intracellular pools mediated by both IP3 and ryanodine receptor calcium channels (Morikawa et al., 2000). The role of calcium pools in frequency and precision control of the DA pacemaker utilising pharmacological tools in perforated-patch recordings is investigated.

Functional Role and Molecular Identity of Store-Operated Channels in DA Neurones?

Based on the previous evidence in DA neurones (Guatteo et al., 1999) and in analogy to other pacemaker systems (Van Goor et al., 1999) store-operated or Gαq-activated channels of the TRPC1-7 gene family (Harteneck et al., 2000) might constitute an important link between intracellular calcium oscillations and membrane excitability. We wish to define their biophysical and pharmacological properties, their regulation by both activation of Gq protein coupled seven-transmembrane receptors and direct store depletion, and their molecular identities by expression profiling of the TRPC gene family. Perforated-patch recordings with unperturbed calcium handling may be essential to investigate a possible functional contribution of TRPC channels to pacemaker activity in DA neurones.

Interaction of Fast and Slow Synaptic Signalling with Pacemaker Activity: Generating Non-Pacemaker Forms of Discharge

The function of DA midbrain neurones is controlled by the interaction of the intrinsic pacemaker mechanism with extrinsic fast (ionotropic) and slow (metabotropic) synaptic input (Kitai et al., 1999). The importance of synaptic integration for DA function maybe best exemplified by the synaptic generation of phasic burst discharge in DA neurones, an activity that dramatically diverges from that of the single spike pacemaker (Overton and Clark, 1997; Lokwan et al., 1999). Burst activity is coupled to dopamine release in a highly non-linear fashion and is believed to be the cellular equivalent for phasic DA signalling in the brain that is critical in reward, cognition and motor learning (Schultz, 1998). How exactly ionotropic GABAergic and glutamatergic synaptic input interacts with slower e.g. muscarinic modulatory inputs and the intrinsic conductances of the pacemaker machinery in DA neurones to generate burst activity is still unclear (Kitai et al., 1999). One facilitating mechanism might be the downregulation of SK channels by modulatory input that—like pharmacological inhibition of SK channels—makes burst firing more likely (Seutin et al., 1993). To understand the mechanisms of this key transition in firing pattern in DA neurones is the central motivation of studying synaptic input of DA neurones. Distinct DA subpopulations in vivo show large differences in their frequency of burst firing (Greenhoff et al., 1988), which might depend both, on differential synaptic input and a different set of somato-dendritic conductances (Neuhoff et al., 2001).

Electrophysiological Characterisation of Glutamatergic and GABAergic Input to Distinct DA Subpopulations:

Based on previous studies of GABAergic (Hausser and Yung, 1994; Tepper et al., 1995; Celada et al., 1999; Schwarzer et al., 2001) and glutamatergic input to DA neurones (Futami et al., 1995; Gotz et al., 1997; Iribe et al., 1999; Chatha et al., 2000), their biophysical and pharmacological properties are studied in different DA subpopulations, which are identified by cell labelling and immunocytochemistry. In light of the different bursting activities of DA neurone populations in vivo (Greenhoff et al., 1988), it may be important to assess whether the DA subpopulations differ in their types of fast synaptic input. In this context, it is important to note that in contrast to NMDAR1, GluR1-4, and GluR7, which are present in all DA midbrain neurones, GluR5 is selectively expressed in SN but absent in VTA DA neurones (Bischoff et al., 1997). Using subunit selective GluR5-antagonists (Bortolotto et al., 1999), the functional difference between glutamatergic input in SN and VTA may be explored.

Functional Interaction of Synaptic Input and Subthreshold Conductances for Timing and Pattern of Discharge in DA Neurones:

There is evidence that synaptically induced action potentials recruit a different set of ion channels compared to those during pacemaker firing (Hausser et al., 2000). In particular, in DA SN neurones these EPSP-mediated action potentials seem to activate less calcium conductances and consequently less calcium-activated potassium channels (Nedergaard, 1999). Subthreshold ion channels like Ih (Magee, 1998, 1999), A-type (Hoffman et al., 1997; Schoppa and Westbrook, 1999) and other potassium channels, sodium and calcium channels have been shown to shape the temporal and spatial integration of synaptic inputs and amplify or filter the inputs in an often non-linear fashion in other neurones (Bennett and Wilson, 1998; Hausser et al., 2001; Stuart and Hausser, 2001; Fricker and Miles, 2002). We have defined significant differences in the repertoire of these channels in distinct DA populations (Neuhoff et al., 2001) they are likely to possess different modes of synaptic integration. Importantly, DA SN neurones possess very efficient dendritic backpropagation of action potentials (Hausser et al., 1995) implicating the somato-dendritic spatial distribution of ion channel species as a significant component. The integration of stimulated and simulated injection of synaptic input are investigated focussing on the conditions of burst initiation combining standard and/or dendritic whole-cell recordings with stimulation of pharmacologically isolated excitatory or inhibitory input (Kitai et al., 1999). We envisage that the construction of computer models of DA neurones with a realistic morphology (Vetter et al., 2001) and biophysically accurate channel mechanisms (Hines and Carnevale, 2000) may help to interpret the experimental data and understand the presumably complex interaction of synaptic and postsynaptic mechanisms in the generation of burst discharge.

Functional Role of Slow Metabotropic Synaptic Mechanisms for Pacemaker and Burst Activity in DA Neurones:

In addition to the interaction of ionotropic synaptic input with somatodendritic ion channels, there is some evidence that the modulation of a subset of these ion channels also plays an important role in pacemaker and burst-activity. It has been noted that selective pharmacological inhibition of SK channels greatly facilitates NMDA-induced burst-like discharge in DA neurones in vitro (Seutin et al., 1993). It has also been suggested that metabotropic receptors like mGluR1 and M1 tune SK activity via controlling intracellular calcium release (Fiorillo and Williams, 1998, 2000). We would like to investigate whether this link is relevant for burst initiation by combining excitatory synaptic stimulation in perforated patch-clamp recordings with pharmacological modulation of muscarinic M1 and mGluR1 receptors. Also, Ih and M-channels are attractive targets for modulation by seven-transmembrane receptors including M1 and 5-HT.

Molecular Anatomy of DA Midbrain Neurones

We have generated a topographic map of distinct DA neuronal phenotypes (Neuhoff et al., 2000, 2001). Based on the well established anatomical and neurochemical topography of dopaminergic projections (Gardner and Ashby, 2000; Joel and Weiner, 2000), our map suggests that the distinct functional DA phenotypes correlate with differential axonal targeting to striatal compartments, nucleus accumbens and prefrontal cortex. However, this possible association needs to be experimentally tested on the single-cell level.

Retrograde Tracing Studies Combined with in vitro Brain Slice Patch-Clamp Recording

To define the axonal target areas of recently identified, functionally and neurochemically distinct DA subpopulations, stereotactically guided retrograde tracer (initially fluorescent beads) are injected in the different target areas of dopaminergic axons starting with the prefrontal cortex, nucleus accumbens (core/shell) and dorsolateral striatum in mice (Mattiace et al., 1989). After 1-2 postoperative days the animals are sacrificed and coronal midbrain slices prepared. Fluorescently labelled individual neurones are identified by fluorescence microscopy. The electrophysiological properties of these DA neurones are studied in patch-clamp recordings and the cells labelled for immunohistochemical processing. This may generate a functional map of DA neurones with identified axonal projections and target the important question to what extent the distinct functional properties of DA neurones (see I & II) are co-segregated with their axonal projections.

In vivo Single-Cell Expression Labelling to Study Functional Properties and Axonal Connectivity of Single-Cell DA Neurones

Although single-cell tracing studies have been carried out by established e.g. juxtacellular methods (Bevan et al., 1998), we see great advantages in introducing GFP-expression vector based i.e. self-amplifying molecular labelling into single DA neurones in vivo. Based on previous transgenic studies (Feng et al., 2000), we can expect a complete but selective labelling of somato-dendritic compartments and the axonal projections of single GFP-expression labelled DA neurones, which may greatly facilitate the detailed anatomical analysis of identified single DA neurones. A a new key technique, the single-cell electroporation-based gene transfer techniques (Haas et al., 2001) may be first established in in vitro brain slices and organotypic slice cultures. Once an optimal electroporation techniques for single DA neurones is established in vitro, it may be possible to apply it in vivo in combination with extracellular or intracellular single-cell recordings. Single-cell in vivo GFP-expression labelling may greatly extend our knowledge of the types of axonal projections and pattern of synaptic connectivity of defined DA subpopulations (e.g. calbindin-positive dorsal tier DA SN neurones). If sucessful, the approach may also be applied to label single neurones in other regions of the basal ganglia

mRNA Expression Profiling for Candidate Genes Controlling the Differential Axonal Targeting and Axonal Branching of DA Neurones

There is convincing evidence that both the selective targeting and branching of axons is orchestrated by a large number of interacting guidance molecules based on attraction and repulsion (Chisholm and Tessier-Lavigne, 1999; Brose and Tessier-Lavigne, 2000). Little is known about the molecular guidance cues that control differential targeting and branching pattern of DA axons to their respective striatal and accumbal compartments and the cortex (Yao et al., 1999). The retrograde tracing studies described in a) may provide information about the functional phenotypes of DA neurones with distinct axonal targets. Based on this information or by direct cytosol harvesting of retrogradely labelled DA neurones, cDNA pools of single DA neurones are generated with defined axonal targets. Single-cell RT-multiplexPCR protocols are generated to probe for the expression profiles for important guidance molecules. These studies include investigation concerning the ephrins and ephrin receptor tyrosine kinase family, as a functional role of differential targeting between mesostriatal and mesoaccumbal DA neurones as well as differential expression of ephrins in striatal matrix and patch compartments has been reported (Janis et al., 1999; Yue et al., 1999). If the initial study is sucessful, the profiling is extended to other main guidance protein families aiming to define differential single-cell mRNA expression profiles that are molecular markers for mesostriatal (patch vs. matrix), mesolimbic (shell vs. core), and mesocortical DA neurones.

Functional and Molecular Plasticity of DA Midbrain Neurones in Mouse Models of Parkinson's Disease

The present invention seeks to provide a precise understanding of the physiology of DA midbrain neurones in the context of their different functional roles in the brain. This knowledge may also provide a detailed framework to identify relevant pathophysiological changes occurring in particular DA subpopulations during brain disease states. These might include altered pacemaker activity, altered synaptic input and integration as well as altered responses to metabolic or modulatory pathways. For a genetic model of dopaminergic neurodegeneration, the weaver mouse, we have demonstrated how dramatic these changes can be (Liss et al., 1999a). Furthermore, the definition of single-cell genotype-phenotype correlations may facilitate the identification of those genes that contribute to the pathophysiology. For the weaver mouse, we have identified a critical role of the SUR1/Kir6.2-mediated K-ATP channel (Liss et al., 1999a). Thus, we believe that a single-cell based analysis of the pathophysiology of brain diseases or chronic conditions that are known to alter DA signalling is a promising strategy to identify relevant cellular mechanisms and correlated genes. This approach would be very useful for mouse models of Parkinson's disease (German et al., 1996), schizophrenia (Mohn et al., 1999; Qiao et al., 2001), and drug addiction (Sora et al., 1998; Zhuang et al., 2001). A chronic neurotoxicological mouse model of PD based on the dopaminergic neurotoxin MPTP (Bezard et al., 1997) can be prepared. Recently improved protocols that show that a chronic MPTP-based mouse model of PD can combine low acute toxicity, selective and progressive DA neurodegeneration, a stable motor phenotype, as well as the formation of inclusion bodies (Petroske et al., 2001) may be used. After establishing the model and the basic analytical tools (i.e. dopamine neurochemistry, TH-immunohistochemistry, stereological non-biased cell counts, rotarod tests), the functional properties of DA neurones in in vitro brain slices during different stages of the PD model in order to identify the chronological order of pathophysiological changes that occur in different types of DA neurones is investigated. Based on previous work (Neuhoff et al., 2000, 2001), the phenotypic plasticity in the four DA population in SN and VTA is compared. The highly vulnerable (ventral tier calbindin-negative DA SN neurones) are compared with the more resistant DA neurones (dorsal tier calbindin-positive DA SN and DA VTA neurones). If functional changes are detected that occur either in degenerating DA neurones or, as compensatory responses, in surviving DA neurones, the genes involved utilising quantitative single-cell mRNA expression profiling and immunocytochemical techniques may be defined. The identification of distinct pathophysiological DA phenotypes that might be correlated with imminent degeneration or compensation and survival are utilised for our comparative genomic approach (see IV).

Based on our previous work (Liss et al., 1999b; Liss et al., 1999a; Liss et al., 2000), we have proposed that differential expression of K-ATP channel subunits is a molecular candidate mechanism for differential vulnerability of DA neurones. For in vivo validation of these concepts, the extent and pattern of DA neurodegeneration in the chronic MPTP mouse model described above comparing the Kir6.2-knockout mouse with wildtyp are studiede. We have already verified the complete loss of plasma-membrane K-ATP channels in DA and hypothalamic neurones (Liss et al., 2000; Miki et al., 2001). If the possible role of Kir6.2-mediated K-ATP channels in selective DA neurodegeneration is confirmed in vivo, a transgenic mouse expressing the K-ATP channel subunit Kir6.2 under a TH promotor is generated and crossed with the Kir6.2 knockout mouse for in vivo rescue experiments (Seino et al., 2000). Also, gene expression in response to mitochondrial dysfunction induced by MPP⁺ or rotenone might be compared in the presence or absence of functional K-ATP channels using DNA array-based methods (see IV) to define the K-ATP channel dependent downstream targets. A knockout mouse model of the presumably mitochondrial K-ATP channel (Kir6.1) may be used for analysis.

Functional Genomics of DA Neurones in Parkinson's Disease

As described above qualitative and quantitative PCR-based single-cell expression profiling techniques is used to identify molecular mechanisms of DA function. Because of the limited number of genes that can be assayed in parallel, their selection has to be directly instructed by experimental observation. However, non-biased wide-scale expression profiling of single cells using DNA array technology, would overcome this limitation. This is a very powerful tool to identify differentially expressed candidate genes in different type of DA neurones that are responsible for their differences in physiology and pathophysiology, in particular the identification of the set of coexpressed genes that render some DA neurones more resistant to neurodegeneration in PD. The limiting factor is not primarily the DNA array technology that is now widely available and well established (Epstein and Butow, 2000; Hughes et al., 2000) but a reliable method of linear amplification of the single cell mRNA or cDNA pool (Kacharmina et al., 1999). With the current array technology, this is necessary to generate sufficient starting material for the hybridisation analysis on DNA arrays (oligonucleotides or cDNA clones) for up to several thousand genes. Recently more reliable single-cell amplification techniques based on several rounds of antisense (a) RNA amplification, as pioneered by Eberwine (Kacharmina et al., 1999) have been reported with a few hundred cells as starting materials (Luo et al., 1999). We have established a reliable protocol for wide-scale expression profiling of single DA neurones. We currently contribute the electrophysiological characterisation, single-cell harvesting, and an optimised protocol for first stand cDNA synthesis, while the company provides their aRNA amplification protocol and performs the DNA array experiments. Our preliminary experiments demonstrated that DNA array results covering >2000 genes from individual DA neurones of the same subpopulation had very similar expression profiles (r>0.9). Based on these findings, experiments are carried out on a wide-scale systematically compare the mRNA expression profiles of different DA subpopulations to identify differentially expressed genes. Initially, calbindin-positive and calbindin negative DA populations may be targeted—as it is possible to identify them on their distinct electrophysiological fingerprint prior to harvesting. This initial set of experiments is likely to identify a number of genes that are differentially expressed in highly vulnerable and resistant DA neurones. In a second step, differential gene expression of DA neurones is investigated with different electrophysiological responses to mitochondrial dysfunction (Liss et al., 1999b; Liss et al., 2000). It is likely that differentially expressed acute response genes contribute to the differential vulnerability of DA neurones. Then the most promising genes are selected and then the validation of their differential expression at the single-cell level is investigated. The type of identified gene (channel, receptor, signal transduction protein, transcription factor, etc.) may predict the strategy for functional analysis.

In addition, the expression of human homologues of the mouse vulnerability genes identified by DNA arrays is investigated. in situ hybridisation, immunocytochemistry and laser-microdissection based RT-PCR analysis (Emmert-Buck et al., 1996; Schutze and Lahr, 1998) of DA neurones from fixed brain sections of control and Parkinsonian brains are used.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be covered by the present invention.

By way of example, it is envisaged that it is now possibe to utilise the findings of the present invention to identify and/or develop agents that are capable of causing a dopaminergic neuron to leave bursting mode and/or of preventing it from entering bursting mode, in the manufacture of a medicament for treating a disorder that is alleviated by reduced dopamine release.

Likewise, it is envisaged that it is now possibe to utilise the findings of the present invention to identify and/or develop agents that are capable of increasing calcium efflux through a calcium channel of a dopaminergic neuron and/or of increasing potassium efflux through a potassium channel of a dopaminergic neuron in the manufacture of a medicament for treating a disorder that is alleviated by reduced dopamine release.

In some cases following administration of one or more agents causing decreased dopamine release from dopaminergic neurons it may be found that dopamine levels are undesirably low or are likely to become so. An agent causing increased dopamine release may then be provided. A kit may be provided comprising, in separate containers, an agent capable of causing increased dopamine release and an agent capable of causing reduced dopamine release. This can be useful in regulating dopamine levels.

It will of course be appreciated that the foregoing discussion in respect of medical uses based upon increased or reduced dopamine release applies mutatis mutandis to methods of treatment. Thus the present invention therefore also includes treatments of the disorders discussed above comprising administering to a human or non-human animal a therapeutically effective amount of one or more agents as described above. Preferably the agents are provided as pharmaceutical compositions.

For these aspects, the above-mentioned general teachings are equally applicable.

### REFERENCE FOR T-TYPE CHANNEL SECTION

Perez-Reyes, E.; Cribbs, L. L.; Daud, A.; Lacerda, A. E.; Barclay, J.; Williamson, M. P.; Fox, M.; Rees, M.; Lee, J. -H.: Molecular characterization of a neuronal low-voltage-activated T-type calcium channel. Nature 391: 896-900, 1998.

### REFERENCES FOR SK3-TYPE CHANNEL SECTION

Austin, C. P.; Holder, D. J.; Ma, L.; Mixson, L. A.; Caskey, C. T.: Mapping of hKCa3 to chromosome 1q21 and investigation of linkage of CAG repeat polymorphism to schizophrenia. Molec. Psychiat. 4: 261-266, 1999.

Bond, C. T.; Sprengel, R.; Bissonnette, J. M.; Kaufmann, W. A.; Pribnow, D.; Neelands, T.; Storck, T.; Baetscher, M.; Jerecic, J.; Maylie, J.; Knaus, H. -G.; Seeburg, P. H.; Adelman, J. P.: Respiration and parturition affected by conditional overexpression of the Ca(2+)-activated K(+) channel subunit, SK3. Science 289: 1942-1946, 2000.

Chandy, K. G.; Fantino, E.; Wittekindt, O.; Kalman, K.; Tong, L. -L.; Ho, T. -H.; Gutman, G. A.; Crocq, M. -A.; Ganguli, R.; Nimgaonkar, V.; Morris-Rosendahl, D. J.; Gargus, J. J. Isolation of a novel potassium channel gene hSKCa3 containing a polymorphic CAG repeat: a candidate for schizophrenia and bipolar disorder? Molec. Psychiat. 3: 32-37, 1998.

Frebourg, T.; Bonnet-Brilhault, F.; Laurent, C.; Campion, D.; Thibaut, F.; Deleuze, J. F.; Petit, M.; Mallet, J.: No evidence for the involvement of the hSKCa3 potassium channel gene in familial and sporadic cases of schizophrenia. (Abstract) Am. J. Hum. Genet. (suppl.) 63: A326 only, 1998.

Kohler, M.; Hirschberg, B.; Bond, C. T.; Kinzie, J. M.; Marrion, N. V.; Maylie, J.; Adelman, J. P.: Small-conductance, calcium-activated potassium channels from mammalian brain. Science 273: 1709-1714, 1996.

Navon, R.; Shamir, E.; Dror, V.; Ghanshani, S.; Litmanovitch, T.; Kimchi, R.; Swartz, M.; Barak, Y.; Fantino, E.; Kalman, K.; Jones, E. G.; Avivi, L.; Chandy, K. G.; Gargus, J. J.; Gutman, G. A.: Strong association between schizophrenia and long CAG repeats in the hKCa3/KCNN3 gene, mapped to lq21, among Israeli Jews. (Abstract) Am. J. Hum. Genet. 63 (suppl.): A337 only, 1998.

Sun, G.; Tomita, H.; Shakkottai, V. G.; Gargus, J. J.: Genomic organization and promoter analysis of human KCNN3 gene. J. Hum. Genet. 46: 463-470, 2001.

Wittekindt, O.; Jauch, A.; Burgert, E.; Scharer, L.; Holtgreve-Grez, H.; Yvert, G.; Imbert, G.; Zimmer, J.; Hoehe, M. R.; Macher, J. -P.; Chiaroni, P.; van Calker, D.; Crocq, M. -A.; Morris-Rosendahl, D. J.: The human small conductance calcium-regulated potassium channel gene (hSKCa3) contains two CAG repeats in exon 1, is on chromosome 1q21.3, and shows a possible association with schizophrenia. Neurogenetics 1: 259-265, 1998.

### REFERENCES FOR DOPAMINERGIC NEURON SECTION A

Barrot M, Calza L, Pozza M, Le Moal M, Piazza P V (2000) Differential calbindin-immunoreactivity in dopamine neurons projecting to the rat striatal complex. Eur J Neurosci 12:4578-4582.

Chiodo L A, Bannon M J, Grace A A, Roth R H, Bunney B S (1984) Evidence for the absence of impulse-regulating somatodendritic and synthesis-modulating nerve terminal autoreceptors on subpopulations of mesocortical dopamine neurons. Neuroscience 12:1-16.

Damier P, Hirsch E C, Agid Y, Graybiel A M (1999a) The substantia nigra of the human brain. I. Nigrosomes and the nigral matrix, a compartmental organization based on calbindin D(28K) immunohistochemistry [see comments]. Brain 122:1421-1436.

Damier P, Hirsch E C, Agid Y, Graybiel A M (1999b) The substantia nigra of the human brain. II. Patterns of loss of dopamine-containing neurons in Parkinson's disease. Brain 122:1437-1448.

Dunnett S B, Bjorklund A (1999) Prospects for new restorative and neuroprotective treatments in Parkinson's disease. Nature 399:A32-39.

Gardner E L, Ashby C R, Jr. (2000) Heterogeneity of the mesotelencephalic dopamine fibers: physiology and pharmacology. Neurosci Biobehav Rev 24:115-118.

Gerfen C R (1992) The neostriatal mosaic: multiple levels of compartmental organization in the basal ganglia. Annu Rev Neurosci 15:285-320.

Goldman-Rakic P S (1999) The physiological approach: functional architecture of working memory and disordered cognition in schizophrenia. Biol Psychiatry 46:650-661.

Gonzalez-Hernandez T, Rodriguez M (2000) Compartmental organization and chemical profile of dopaminergic and GABAergic neurons in the substantia nigra of the rat. J Comp Neurol 421:107-135.

Grace A A, Onn S P (1989) Morphology and electrophysiological properties of immunocytochemically identified rat dopamine neurons recorded in vitro. J Neurosci 9:3463-3481.

Greenhoff J, Ugedo L, Svensson T H (1988) Firing pattern of midbrain dopamine neurons: differences between A9 and A10 cells. Acta Physiol Scand 134:127-132.

Groenewegen H J, Wright C I, Beijer A V, Voorn P (1999) Convergence and segregation of ventral striatal inputs and outputs. Ann N Y Acad Sci 877:49-63.

Haber S N, Fudge J L, McFarland N R (2000) Striatonigrostriatal pathways in primates form an ascending spiral from the shell to the dorsolateral striatum. J Neurosci 20:2369-2382.

Joel D, Weiner I (2000) The connections of the dopaminergic system with the striatum in rats and primates: an analysis with respect to the functional and compartmental organization of the striatum. Neuroscience 96:451-474.

Kitai S T, Shepard P D, Callaway J C, Scroggs R (1999) Afferent modulation of dopamine neuron firing patterns. Curr Opin Neurobiol 9:690-697.

Lacey M G, Mercuri N B, North R A (1989) Two cell types in rat substantia nigra zona compacta distinguished by membrane properties and the actions of dopamine and opioids. J Neurosci 9:1233-1241.

Liang C L, Sinton C M, German D C (1996) Midbrain dopaminergic neurons in the mouse: co-localization with Calbindin-D28K and calretinin. Neuroscience 75:523-533.

Maurin Y, Banrezes B, Menetrey A, Mailly P, Deniau J M (1999) Three-dimensional distribution of nigrostriatal neurons in the rat: relation to the topography of striatonigral projections. Neuroscience 91:891-909.

Paladini C A, Celada P, Tepper J M (1999) Striatal, pallidal, and pars reticulata evoked inhibition of nigrostriatal dopaminergic neurons is mediated by GABA(A) receptors in vivo. Neuroscience 89:799-812.

Pucak M L, Grace AA (1994) Regulation of substantia nigra dopamine neurons. Crit Rev Neurobiol 9:67-89.

Richards C D, Shiroyama T, Kitai S T (1997) Electrophysiological and immunocytochemical characterization of GABA and dopamine neurons in the substantia nigra of the rat. Neuroscience 80:545-557.

Sanghera M K, Trulson M E, German D C (1984) Electrophysiological properties of mouse dopamine neurons: in vivo and in vitro studies. Neuroscience 12:793-801.

Shepard P D, German D C (1988) Electrophysiological and pharmacological evidence for the existence of distinct subpopulations of nigrostriatal dopaminergic neuron in the rat. Neuroscience 27:537-546.

Spanagel R, Weiss F (1999) The dopamine hypothesis of reward: past and current status. Trends Neurosci 22:521-527.

Svensson T H (2000) Dysfunctional brain dopamine systems induced by psychotomimetic NMDA-receptor antagonists and the effects of antipsychotic drugs. Brain Res Bain Res Rev 31:320-329.

Tan Y, Williams E A, Lancia A J, Zahm D S (2000) On the altered expression of tyrosine hydroxylase and calbindin-D 28 kD immunoreactivities and viability of neurons in the ventral tegmental area of Tsai following injections of 6-hydroxydopamine in the medial forebrain bundle in the rat. Brain Res 869:56-68.

Tzschentke T M (2001) Pharmacology and behavioral pharmacology of the mesocortical dopamine system. Prog Neurobiol 63:241-320.

Verhoeff N P (1999) Radiotracer imaging of dopaminergic transmission in neuropsychiatric disorders. Psychopharmacology (Berl) 147:217-249.

### REFERENCES FOR EXPERIMENTAL SECTION A

1. J. Wolfart, H. Neuhoff, O. Franz, J. Roeper, J Neurosci 21, 3443-56. (2001).

2. S. E. Bowden, S. Fletcher, D. J. Loane, N. V. Marrion, J Neurosci 21, RC175. (2001).

3. D. L. Cardozo, B. P. Bean, J Neurophysiol 74, 1137-48 (1995).

4. Y. Kang, S. T. Kitai, Neurosci Res 18, 209-21 (1993b).

5. V. Seutin, F. Mkahli, L. Massotte, A. Dresse, J Neurophysiol 83, 192-7 (2000).

6. A. A. Grace, B. S. Bunney, J Neurosci 4, 2877-90 (1984b).

7. S. W. Johnson, V. Seutin, R. A. North, Science 258, 665-7 (1992).

8. S. T. Kitai, P. D. Shepard, J. C. Callaway, R. Scroggs, Curr Opin Neurobiol 9, 690-7 (1999).

9. P. D. Shepard, B. S. Bunney, Brain Res 463, 380-4 (1988).

10. D. A. McCormick, T. Bal, Annu Rev Neurosci 20, 185-215 (1997).

11. S. J. Korn, R. Horn, J Gen Physiol 94, 789-812. (1989).

12. C. R. Legendy, M. Salcman, J Neurophysiol 53, 926-39 (1985).

### REFERENCES FOR EXPERIMENTAL SECTION B

Amini B, Clark J W, Jr., Canavier C C (1999) Calcium dynamics underlying pacemaker-like and burst firing oscillations in midbrain dopaminergic neurons: a computational study. J Neurophysiol 82:2249-2261.

Betarbet R, Sherer T B, MacKenzie G, Garcia-Osuna M, Panov A V, Greenamyre J T (2000) Chronic systemic pesticide exposure reproduces features of Parkinson's disease. Nat Neurosci 3:1301-1306.

Bond C T, Maylie J, Adelman J P (1999) Small-conductance calcium-activated potassium channels. Ann N Y Acad Sci 868:370-378.

Damier P, Hirsch E C, Agid Y, Graybiel A M (1999a) The substantia nigra of the human brain. II. Patterns of loss of dopamine-containing neurons in Parkinson's disease. Brain 122:1437-1448.

Damier P, Hirsch E C, Agid Y, Graybiel A M (1999b) The substantia nigra of the human brain. I. Nigrosomes and the nigral matrix, a compartmental organization based on calbindin D(28K) immunohistochemistry. Brain 122:1421-1436.

Dunnett S B, Bjorklund A (1999) Prospects for new restorative and neuroprotective treatments in Parkinson's disease. Nature 399:A32-39.

Franz O, Liss B, Neu A, Roeper J (2000) Single-cell mRNA expression of HCN1 correlates with a fast gating phenotype of hyperpolarization-activated cyclic nucleotide-gated ion channels (Ih) in central neurons. Eur J Neurosci 12:2685-2693.

German D C, Nelson E L, Liang C L, Speciale S G, Sinton C M, Sonsalla P K (1996) The neurotoxin MPTP causes degeneration of specific nucleus A8, A9 and A10 dopaminergic neurons in the mouse. Neurodegeneration 5:299-312.

Goldman-Rakic PS (1999) The physiological approach: functional architecture of working memory and disordered cognition in schizophrenia. Biol Psychiatry 46:650-661.

Grace A A (1991) Regulation of spontaneous activity and oscillatory spike firing in rat midbrain dopamine neurons recorded in vitro. Synapse 7:221-234.

Grace A A, Onn S P (1989) Morphology and electrophysiological properties of immunocytochemically identified rat dopamine neurons recorded in vitro. J Neurosci 9:3463-3481.

Hattori N, Shimura H, Kubo S, Wang M, Shimizu N, Tanaka K, Mizuno Y (2000) Importance of familial Parkinson's disease and parkinsonism to the understanding of nigral degeneration in sporadic Parkinson's disease. J Neural Transm Suppl 60:101-116.

Kitai S T, Shepard P D, Callaway J C, Scroggs R (1999) Afferent modulation of dopamine neuron firing patterns. Curr Opin Neurobiol 9:690-697.

Kosel S, Hofhaus G, Maassen A, Vieregge P, Graeber M B (1999) Role of mitochondria in Parkinson disease. Biol Chem 380:865-870.

Lacey M G, Mercuri N B, North R A (1989) Two cell types in rat substantia nigra zona compacta distinguished by membrane properties and the actions of dopamine and opioids. J Neurosci 9:1233-1241.

Liss B, Neu A, Roeper J (1999a) The weaver mouse gain-of-function phenotype of dopaminergic midbrain neurons is determined by coactivation of wvGirk2 and K-ATP channels. J Neurosci 19:8839-8848.

Liss B, Bruns R, Roeper J (1999b) Alternative sulfonylurea receptor expression defines metabolic sensitivity of K-ATP channels in dopaminergic midbrain neurons. Embo J 18:833-846.

Liss B, Franz O, Neuhoff H, Roeper J (2001) Tuning the pacemaker frequency of individual dopaminergic neurons by variable Kv4.3 transcript numbers: a quantitative single-cell real-time PCR study. Soc Neurosci Abstr 27.

Liss B, Neuhoff H, Miki T, Seino S, Roeper J (2000) MPP+ selectively activates SUR1/Kir6.2 K-ATP channels in dopaminergic midbrain neurons: direct evidence from Kir6.2 knockout mouse. Soc Neurosci Abstr 26.

Miki T, Liss B, Minami K, Shiuchi T, Saraya A, Kashima Y, Horiuchi M, Ashcroft F, Minokoshi Y, Roeper J, Seino S (2001) ATP-sensitive potassium channels in hypothalamic neurons are essential for the maintenance of glucose homeostasis. Nature Neuroscience 4:507-512.

Neu A, Neuhoff H, Franzl, Ullrich K, Isbrandt D, Roeper J (2000) New insights in the Pathophysiology of Guanidinoacetate Methyltransferase (GAMT) Deficiency: the accummulating metabolite guanidinoacetate acts as a selective GABA-A receptor agonist. Soc Neurosci Abstr 26.

Neuhoff H, Neu A, Liss B, Roeper J (2000) Molecular and functional topography of mouse midbrain neurons. Soc Neurosci Abstr 26.

Neuhoff H, Neu A, Liss B, Roeper J (2001) Ih and IA channels spape the functional topography of dopaminergic midbrain neurons. Soc Neurosci Abstr 27.

Richards C D, Shiroyama T, Kitai S T (1997) Electrophysiological and immunocytochemical characterization of GABA and dopamine neurons in the substantia nigra of the rat. Neuroscience 80:545-557.

Spanagel R, Weiss F (1999) The dopamine hypothesis of reward: past and current status. Trends Neurosci 22:521-527.

Svensson T H (2000) Dysfunctional brain dopamine systems induced by psychotomimetic NMDA-receptor antagonists and the effects of antipsychotic drugs. Brain Res Bain Res Rev 31:320-329.

Szczypka M S, Rainey M A, Palmiter R D (2000) Dopamine is required for hyperphagia in Lep(ob/ob) mice. Nat Genet 25:102-104.

Verhoeff N P (1999) Radiotracer imaging of dopaminergic transmission in neuropsychiatric disorders. Psychopharmacology (Berl) 147:217-249.

Wolfart J, Neuhoff H, Franz O, Roeper J (2001) Differential expression of the small-conductance, calcium activated potassium channel SK3 is critical for pacemaker control in dopaminergic midbrain neurons. J Neurosci in press.

Yung W H, Hausser M A, Jack J J (1991) Electrophysiology of dopaminergic and non-dopaminergic neurones of the guinea-pig substantia nigra pars compacta in vitro. J Physiol (Lond) 436:643-667.

Zhou Q Y, Palmiter R D (1995) Dopamine-deficient mice are severely hypoactive, adipsic, and aphagic. Cell 83:1197-1209.

### REFERENCES FOR EXPERIMENTAL SECTION C

Amini B, Clark J W, Jr., Canavier C C (1999) Calcium dynamics underlying pacemaker-like and burst firing oscillations in midbrain dopaminergic neurons: a computational study. J Neurophysiol 82:2249-2261.

An W F, Bowlby M R, Betty M, Cao J, Ling H P, Mendoza G, Hinson J W, Mattsson K I, Strassle B W, Trimmer J S, Rhodes K J (2000) Modulation of A-type potassium channels by a family of calcium sensors. Nature 403:553-556.

Barrot M, Calza L, Pozza M, Le Moal M, Piazza P V (2000) Differential calbindin-immunoreactivity in dopamine neurons projecting to the rat striatal complex. Eur J Neurosci 12:4578-4582.

Bennett B D, Wilson C J (1998) Synaptic regulation of action potential timing in neostriatal cholinergic interneurons. J Neurosci 18:8539-8549.

Betarbet R, Sherer T B, MacKenzie G, Garcia-Osuna M, Panov A V, Greenamyre J T (2000) Chronic systemic pesticide exposure reproduces features of Parkinson's disease. Nat Neurosci 3:1301-1306.

Bevan M D, Booth P A, Eaton S A, Bolam J P (1998) Selective innervation of neostriatal interneurons by a subclass of neuron in the globus pallidus of the rat. J Neurosci 18:9438-9452.

Bezard E, Dovero S, Bioulac B, Gross C (1997) Effects of different schedules of MPTP administration on dopaminergic neurodegeneration in mice. Exp Neurol 148:288-292.

Bischoff S, Barhanin J, Bettler B, Mulle C, Heinemann S (1997) Spatial distribution of kainate receptor subunit mRNA in the mouse basal ganglia and ventral mesencephalon. J Comp Neurol 379:541-562.

Bortolotto Z A, Clarke V R, Delany C M, Parry M C, Smolders I, Vignes M, Ho K H, Miu P, Brinton B T, Fantaske R, Ogden A, Gates M, Ornstein P L, Lodge D, Bleakman D, Collingridge G L (1999) Kainate receptors are involved in synaptic plasticity. Nature 402:297-301.

Brose K, Tessier-Lavigne M (2000) Slit proteins: key regulators of axon guidance, axonal branching, and cell migration. Curr Opin Neurobiol 10:95-102.

Cardozo D L, Bean B P (1995) Voltage-dependent calcium channels in rat midbrain dopamine neurons: modulation by dopamine and GABAB receptors. J Neurophysiol 74:1137-1148.

Catterall W A (1998) Structure and function of neuronal Ca2+ channels and their role in neurotransmitter release. Cell Calcium 24:307-323.

Celada P, Paladini C A, Tepper J M (1999) GABAergic control of rat substantia nigra dopaminergic neurons: role of globus pallidus and substantia nigra pars reticulate. Neuroscience 89:813-825.

Chatha B T, Bernard V, Streit P, Bolam J P (2000) Synaptic localization of ionotropic glutamate receptors in the rat substantia nigra. Neuroscience 101:1037-1051.

Chiodo L A, Bannon M J, Grace A A, Roth R H, Bunney B S (1984) Evidence for the absence of impulse-regulating somatodendritic and synthesis-modulating nerve terminal autoreceptors on subpopulations of mesocortical dopamine neurons. Neuroscience 12:1-16.

Chisholm A, Tessier-Lavigne M (1999) Conservation and divergence of axon guidance mechanisms. Curr Opin Neurobiol 9:603-615.

Damier P, Hirsch E C, Agid Y, Graybiel A M (1999a) The substantia nigra of the human brain. I. Nigrosomes and the nigral matrix, a compartmental organization based on calbindin D(28K) immunohistochemistry [see comments]. Brain 122:1421-1436.

Damier P, Hirsch E C, Agid Y, Graybiel A M (1999b) The substantia nigra of the human brain. II. Patterns of loss of dopamine-containing neurons in Parkinson's disease. Brain 122:1437-1448.

Dunnett S B, Bjorklund A (1999) Prospects for new restorative and neuroprotective treatments in Parkinson's disease. Nature 399:A32-39.

Emmert-Buck M R, Bonner R F, Smith P D, Chuaqui R F, Zhuang Z, Goldstein S R, Weiss R A, Liotta L A (1996) Laser capture microdissection. Science 274:998-1001.

Epstein C B, Butow R A (2000) Microarray technology—enhanced versatility, persistent challenge. Curr Opin Biotechnol 11:36-41.

Feng G, Mellor R H, Bernstein M, Keller-Peck C, Nguyen Q T, Wallace M, Nerbonne J M, Lichtman J W, Sanes J R (2000) Imaging neuronal subsets in transgenic mice expressing multiple spectral variants of GFP. Neuron 28:41-51.

Fiorillo C D, Williams J T (1998) Glutamate mediates an inhibitory postsynaptic potential in dopamine neurons. Nature 394:78-82.

Fiorillo C D, Williams J T (2000) Cholinergic inhibition of ventral midbrain dopamine neurons. J Neurosci 20:7855-7860.

Franz O, Liss B, Neu A, Roeper J (2000) Single-cell mRNA expression of HCN1 correlates with a fast gating phenotype of hyperpolarization-activated cyclic nucleotide-gated ion channels (Ih) in central neurons. Eur J Neurosci 12:2685-2693.

Fricker D, Miles R (2002) EPSP amplification and the precision of spike timing in bippocampal neurons. Neuron 28:559-569.

Futami T, Takakusaki K, Kitai S T (1995) Glutamatergic and cholinergic inputs from the pedunculopontine tegmental nucleus to dopamine neurons in the substantia nigra pars compacta. Neurosci Res 21:331-342.

Gardner E L, Ashby C R, Jr. (2000) Heterogeneity of the mesotelencephalic dopamine fibers: physiology and pharmacology. Neurosci Biobehav Rev 24:115-118.

Gariano R F, Tepper J M, Sawyer S F, Young S J, Groves P M (1989) Mesocortical dopaminergic neurons. 1. Electrophysiological properties and evidence for soma-dendritic autoreceptors. Brain Res Bull 22:511-516.

Gerfen C R (1992) The neostriatal mosaic: multiple levels of compartmental organization in the basal ganglia. Annu Rev Neurosci 15:285-320.

German D C, Nelson E L, Liang C L, Speciale S G, Sinton C M, Sonsalla P K (1996) The neurotoxin MPTP causes degeneration of specific nucleus A8, A9 and A10 dopaminergic neurons in the mouse. Neurodegeneration 5:299-312.

Goldman-Rakic P S (1999) The physiological approach: functional architecture of working memory and disordered cognition in schizophrenia. Biol Psychiatry 46:650-661.

Gonzalez-Hernandez T, Rodriguez M (2000) Compartmental organization and chemical profile of dopaminergic and GABAergic neurons in the substantia nigra of the rat. J Comp Neurol 421:107-135.

Goodchild J (2000) Hammerhead ribozymes: biochemical and chemical considerations. Curr Opin Mol Ther 2:272-281.

Gotz T, Kraushaar U, Geiger J, Lubke J, Berger T, Jonas P (1997) Functional properties of AMPA and NMDA receptors expressed in identified types of basal ganglia neurons. J Neurosci 17:204-215.

Grace A A, Onn S P (1989) Morphology and electrophysiological properties of immunocytochemically identified rat dopamine neurons recorded in vitro. J Neurosci 9:3463-3481.

Greenhoff J, Ugedo L, Svensson T H (1988) Firing pattern of midbrain dopamine neurons: differences between A9 and A10 cells. Acta Physiol Scand 134:127-132.

Groenewegen H J, Wright C I, Beijer A V, Voorn P (1999) Convergence and segregation of ventral striatal inputs and outputs. Ann N Y Acad Sci 877:49-63.

Guatteo E, Mercuri N B, Bernardi G, Knopfel T (1999) Group I metabotropic glutamate receptors mediate an inward current in rat substantia nigra dopamine neurons that is independent from calcium mobilization. J Neurophysiol 82:1974-1981.

Haas K, Sin W, Javaherian A, Li Z, Cline H T (2001) Single-cell electroporation for gene transfer in vivo. Neuron 29:583-591.

Haber S N, Fudge J L, McFarland N R (2000) Striatonigrostriatal pathways in primates form an ascending spiral from the shell to the dorsolateral striatum. J Neurosci 20:2369-2382.

Harteneck C, Plant T D, Schultz G (2000) From worm to man: three subfamilies of TRP channels. Trends Neurosci 23:159-166.

Hausser M, Spruston N, Stuart G J (2000) Diversity and dynamics of dendritic signaling. Science 290:739-744.

Hausser M, Major G, Stuart G J (2001) Differential shunting of EPSPs by action potentials. Science 291:138-141.

Hausser M, Stuart G, Racca C, Sakmann B (1995) Axonal initiation and active dendritic propagation of action potentials in substantia nigra neurons. Neuron 15:637-647.

Hausser M A, Yung W H (1994) Inhibitory synaptic potentials in guinea-pig substantia nigra dopamine neurones in vitro. J Physiol 479:401-422.

Hines M L, Carnevale N T (2000) Expanding NEURON's repertoire of mechanisms with NMODL. Neural Comput 12:995-1007.

Hoffman D A, Magee J C, Colbert C M, Johnston D (1997) K+ channel regulation of signal propagation in dendrites of hippocampal pyramidal neurons [see comments]. Nature 387:869-875.

Hughes T R, Marton M J, Jones A R, Roberts C J, Stoughton R, Armour C D, Bennett H A, Coffey E, Dai H, He Y D, Kidd M J, King A M, Meyer M R, Slade D, Lum P Y, Stepaniants S B, Shoemaker D D, Gachotte D, Chakraburtty K, Simon J, Bard M, Friend S H (2000) Functional discovery via a compendium of expression profiles. Cell 102:109-126.

Iribe Y, Moore K, Pang K C, Tepper J M (1999) Subthalamic stimulation-induced synaptic responses in substantia nigra pars compacta dopaminergic neurons in vitro. J Neurophysiol 82:925-933.

Janis L S, Cassidy R M, Kromer L F (1999) Ephrin-A binding and EphA receptor expression delineate the matrix compartment of the striatum. J Neurosci 19:4962-4971.

Joel D, Weiner I (2000) The connections of the dopaminergic system with the striatum in rats and primates: an analysis with respect to the functional and compartmental organization of the striatum. Neuroscience 96:451-474.

Kacharmina J E, Crino P B, Eberwine J (1999) Preparation of cDNA from single cells and subcellular regions. Methods Enzymol 303:3-18.

Kang Y, Kitai S T (1993b) A whole cell patch-clamp study on the pacemaker potential in dopaminergic neurons of rat substantia nigra compacta. Neurosci Res 18:209-221.

Kitai S T, Shepard P D, Callaway J C, Scroggs R (1999) Afferent modulation of dopamine neuron firing patterns. Curr Opin Neurobiol 9:690-697.

Krimer L S, Muly E C, 3rd, Williams G V, Goldman-Rakic P S (1998) Dopaminergic regulation of cerebral cortical microcirculation [see comments]. Nat Neurosci 1:286-289.

Lacey M G, Mercuri N B, North R A (1989) Two cell types in rat substantia nigra zona compacta distinguished by membrane properties and the actions of dopamine and opioids. J Neurosci 9:1233-1241.

Lacinova L, Klugbauer N, Hofmann F (2000) Low voltage activated calcium channels: from genes to function. Gen Physiol Biophys 19:121-136.

Liang C L, Sinton C M, German D C (1996) Midbrain dopaminergic neurons in the mouse: co-localization with Calbindin-D28K and calretinin. Neuroscience 75:523-533.

Liss B, Neu A, Roeper J (1999a) The weaver mouse gain-of-function phenotype of dopaminergic midbrain neurons is determined by coactivation of wvGirk2 and K-ATP channels. J Neurosci 19:8839-8848.

Liss B, Bruns R, Roeper J (1999b) Alternative sulfonylurea receptor expression defines metabolic sensitivity of K-ATP channels in dopaminergic midbrain neurons. Embo J 18:833-846.

Liss B, Franz O, Neuhoff H, Roeper J (2001) Tuning the pacemaker frequency of individual dopaminergic neurons by variable Kv4.3 transcript numbers: a quantitative single-cell real-time PCR study. Soc Neurosci Abstr 27.

Liss B, Neuhoff H, Miki T, Seino S, Roeper J (2000) MPP+ selectively activates SUR1/Kir6.2 K-ATP channels in dopaminergic midbrain neurons: direct evidence from Kir6.2 knockout mouse. Soc Neurosci Abstr 26.

Liu R, Li W, Karin N J, Bergh J J, Adler-Storthz K, Farach-Carson M C (2000) Ribozyme ablation demonstrates that the cardiac subtype of the voltage-sensitive calcium channel is the molecular transducer of 1,25-dihydroxyvitamin D(3)-stimulated calcium influx in osteoblastic cells. J Biol Chem 275:8711-8718.

Lokwan S J, Overton P G, Berry M S, Clark D (1999) Stimulation of the pedunculopontine tegmental nucleus in the rat produces burst firing in A9 dopaminergic neurons. Neuroscience 92:245-254.

Luo L, Salunga R C, Guo H, Bittner A, Joy K C, Galindo J E, Xiao H, Rogers K E, Wan J S, Jackson M R, Erlander M G (1999) Gene expression profiles of laser-captured adjacent neuronal subtypes. Nat Med 5:117-122.

Magee J C (1998) Dendritic hyperpolarization-activated currents modify the integrative properties of hippocampal CA1 pyramidal neurons. J Neurosci 18:7613-7624.

Magee J C (1999) Dendritic Ih normalizes temporal summation in hippocampal CA1 neurons. Nat Neurosci 2:848.

Marrion N V, Tavalin S J (1998) Selective activation of Ca2+-activated K+ channels by co-localized Ca2+ channels in hippocampal neurons. Nature 395:900-905.

Mattiace L A, Baring M D, Manaye K F, Mihailoff G A, German D C (1989) Mesostriatal projections in BALB/c and CBA mice: a quantitative retrograde neuroanatomical tracing study. Brain Res Bull 23:61-68.

Maurin Y, Banrezes B, Menetrey A, Mailly P, Deniau J M (1999) Three-dimensional distribution of nigrostriatal neurons in the rat: relation to the topography of striatonigral projections. Neuroscience 91:891-909.

Miki T, Liss B, Minami K, Shiuchi T, Saraya A, Kashima Y, Horiuchi M, Ashcroft F, Minokoshi Y, Roeper J, Seino S (2001) ATP-sensitive potassium channels in hypothalamic neurons are essential for the maintenance of glucose homeostasis. Nature Neuroscience 4:in press.

Mohn A R, Gainetdinov R R, Caron M G, Koller B H (1999) Mice with reduced NMDA receptor expression display behaviors related to schizophrenia. Cell 98:427-436.

Morikawa H, Imani F, Khodakhah K, Williams J T (2000) Inositol 1,4,5-triphosphate-evoked responses in midbrain dopamine neurons. J Neurosci 20:RC103.

Nedergaard S (1999) Regulation of action potential size and excitability in substantia nigra compacta neurons: sensitivity to 4-aminopyridine. J Neurophysiol 82:2903-2913.

Nedergaard S, Flatman J A, Engberg I (1993) Nifedipine- and omega-conotoxin-sensitive Ca2+ conductances in guinea-pig substantia nigra pars compacta neurones. J Physiol (Lond) 466:727-747.

Neuhoff H, Neu A, Liss B, Roeper J (2000) Molecular and functional topography of mouse midbrain neurons. Soc Neurosci Abstr 26.

Neuhoff H, Neu A, Liss B, Roeper J (2001) Ih and IA channels spape the functional topography of dopaminergic midbrain neurons. Soc Neurosci Abstr 27.

Overton P G, Clark D (1997) Burst firing in midbrain dopaminergic neurons. Brain Res Brain Res Rev 25:312-334.

Paladini C A, Celada P, Tepper J M (1999) Striatal, pallidal, and pars reticulata evoked inhibition of nigrostriatal dopaminergic neurons is mediated by GABA(A) receptors in vivo. Neuroscience 89:799-812.

Petroske E, Meredith G E, Callen S, Totterell S, Lau Y-S (2001) Mouse model of parkinsonism: a comparison between subacute MPTP and chronic MPTP/probenecid treatment. Neuroscience in press.

Platzer J, Engel J, Schrott-Fischer A, Stephan K, Bova S, Chen H, Zheng H, Striessnig J (2000) Congenital deafness and sinoatrial node dysfunction in mice lacking class D L-type Ca2+ channels. Cell 102:89-97.

Pucak M L, Grace A A (1994) Regulation of substantia nigra dopamine neurons. Crit Rev Neurobiol 9:67-89.

Qiao H, Noda Y, Kamei H, Nagai T, Furukawa H, Miura H, Kayukawa Y, Ohta T, Nabeshima T (2001) Clozapine, but not haloperidol, reverses social behavior deficit in mice during withdrawal from chronic phencyclidine treatment. Neuroreport 12:11-15.

Richards C D, Shiroyama T, Kitai S T (1997) Electrophysiological and immunocytochemical characterization of GABA and dopamine neurons in the substantia nigra of the rat. Neuroscience 80:545-557.

Sanghera M K, Trulson M E, German D C (1984) Electrophysiological properties of mouse dopamine neurons: in vivo and in vitro studies. Neuroscience 12:793-801.

Schoppa N E, Westbrook G L (1999) Regulation of synaptic timing in the olfactory bulb by an A-type potassium current. Nat Neurosci 2:1106-1113.

Schultz W (1998) Predictive reward signal of dopamine neurons. J Neurophysiol 80:1-27.

Schutze K, Lahr G (1998) Identification of expressed genes by laser-mediated manipulation of single cells. Nat Biotechnol 16:737-742.

Schwarzer C, Berresheim U, Pirker S, Wieselthaler A, Fuchs K, Sieghart W, Sperk G (2001) Distribution of the major gamma-aminobutyric acid (A) receptor subunits in the basal ganglia and associated limbic brain areas of the adult rat. J Comp Neurol 433:526-549.

Seino S, Iwanaga T, Nagashima K, Miki T (2000) Diverse roles of K(ATP) channels learned from Kir6.2 genetically engineered mice. Diabetes 49:311-318.

Seutin V, Johnson S W, North R A (1993) Apamin increases NMDA-induced burst-firing of rat mesencephalic dopamine neurons. Brain Res 630:341-344.

Seutin V, Mkahli F, Massotte L, Dresse A (2000) Calcium release from internal stores is required for the generation of spontaneous hyperpolarizations in dopaminergic neurons of neonatal rats. J Neurophysiol 83:192-197.

Shepard P D, German D C (1988) Electrophysiological and pharmacological evidence for the existence of distinct subpopulations of nigrostriatal dopaminergic neuron in the rat. Neuroscience 27:537-546.

Sora I, Wichems C, Takahashi N, Li X F, Zeng Z, Revay R, Lesch K P, Murphy D L, Uhl G R (1998) Cocaine reward models: conditioned place preference can be established in dopamine- and in serotonin-transporter knockout mice. Proc Natl Acad Sci USA 95:7699-7704.

Spanagel R, Weiss F (1999) The dopamine hypothesis of reward: past and current status. Trends Neurosci 22:521-527.

Strubing C, Krapivinsky G, Krapivinsky L, Clapham DE (2001) Trpc1 and trpc5 form a novel cation channel in mammalian brain. Neuron 29:645-655.

Stuart G J, Hausser M (2001) Dendritic coincidence detection of EPSPs and action potentials. Nat Neurosci 4:63-71.

Svensson T H (2000) Dysfunctional brain dopamine systems induced by psychotomimetic NMDA-receptor antagonists and the effects of antipsychotic drugs. Brain Res Bain Res Rev 31:320-329.

Szczypka M S, Rainey M A, Palmiter R D (2000) Dopamine is required for hyperphagia in Lep(ob/ob) mice. Nat Genet 25:102-104.

Takada M, Kang Y, Imanishi M (2001) Immunohistochemical localization of voltage-gated calcium channels in substantia nigra dopamine neurons. Eur J Neurosci 13:757-762.

Tan Y, Williams E A, Lancia A J, Zahm D S (2000) On the altered expression of tyrosine hydroxylase and calbindin-D 28 kD immunoreactivities and viability of neurons in the ventral tegmental area of Tsai following injections of 6-hydroxydopamine in the medial forebrain bundle in the rat. Brain Res 869:56-68.

Tepper J M, Martin L P, Anderson D R (1995) GABAA receptor-mediated inhibition of rat substantia nigra dopaminergic neurons by pars reticulata projection neurons. J Neurosci 15:3092-3103.

Tzschentke T M (2001) Pharmacology and behavioral pharmacology of the mesocortical dopamine system. Prog Neurobiol 63:241-320.

Van Goor F, Krsmanovic L Z, Catt K J, Stojilkovic S S (1999) Coordinate regulation of gonadotropin-releasing hormone neuronal firing patterns by cytosolic calcium and store depletion. Proc Natl Acad Sci USA 96:4101-4106.

Verhoeff N P (1999) Radiotracer imaging of dopaminergic transmission in neuropsychiatric disorders. Psychopharmacology (Berl) 147:217-249.

Vetter P, Roth A, Hausser M (2001) Propagation of action potentials in dendrites depends on dendritic morphology. J Neurophysiol 85:926-937.

Wang G J, Volkow N D, Logan J, Pappas N R, Wong C T, Zhu W, Netusil N, Fowler J S (2001) Brain dopamine and obesity. Lancet 357:354-357.

Wilson C J, Callaway J C (2000) Coupled oscillator model of the dopaminergic neuron of the substantia nigra. J Neurophysiol 83:3084-3100.

Wolfart J, Roeper J (2001) Preferential coupling of small-conductance, calcium-activated potassium channels to Ni+-sensitvie calcium channels in dopaminergic midbrain neurons. Soc Neurosci Abstr 27.

Wolfart J, Neuhoff H, Franz O, Roeper J (2001) Differential expression of the small-conductance, calcium activated potassium channel SK3 is critical for pacemaker control in dopaminergic midbrain neurons. J Neurosci in press.

Yao J A, Jiang M, Fan J S, Zhou Y Y, Tseng G N (1999) Heterogeneous changes in K currents in rat ventricles three days after myocardial infarction. Cardiovasc Res 44:132-145.

Yue Y, Widmer D A, Halladay A K, Cerretti D P, Wagner G C, Dreyer J L, Zhou R (1999) Specification of distinct dopaminergic neural pathways: roles of the Eph family receptor EphB1 and ligand ephrin-B2. J Neurosci 19:2090-2101.

Yung W H, Hausser M A, Jack J J (1991) Electrophysiology of dopaminergic and non-dopaminergic neurones of the guinea-pig substantia nigra pars compacta in vitro. J Physiol (Lond) 436:643-667.

Zhou Q Y, Palmiter R D (1995) Dopamine-deficient mice are severely hypoactive, adipsic, and aphagic. Cell 83:1197-1209.

Zhuang X, Oosting R S, Jones S R, Gainetdinov R R, Miller G W, Caron M G, Hen R (2001) Hyperactivity and impaired response habituation in hyperdopaminergic mice. Proc Natl Acad Sci USA 98:1982-1987.

1. Use of an agent in the manufacture of a medicament, wherein said agent is capable of causing a dopaminergic neuron to enter bursting mode and/or of preventing it from leaving bursting mode.

2. Use of an agent in the manufacture of a medicament, wherein said agent is capable of affecting the firing mode of a dopaminergic neuron that causes a beneficial change in dopamine release from said dopaminergic neuron.

3. Use of an agent in the manufacture of a medicament, wherein said agent is capable of affecting the firing mode of a dopaminergic neuron that causes a beneficial increase in dopamine release from said dopaminergic neuron.

4. Use of an agent in the manufacture of a medicament, wherein said agent is capable of causing a dopaminergic neuron to enter bursting mode and/or of preventing it from leaving bursting mode, wherein said agent modulates: a T-type channel and/or an SK channel and/or the coupling of a T-type channel with an SK channel.

5. Use of an agent in the manufacture of a medicament, wherein said agent is capable of affecting the firing mode of a dopaminergic neuron that causes a beneficial change in dopamine release from said dopaminergic neuron, wherein said agent modulates: a T-type channel and/or an SK channel and/or the coupling of a T-type channel with an SK channel.

6. Use of an agent in the manufacture of a medicament, wherein said agent is capable of affecting the firing mode of a dopaminergic neuron that causes a beneficial increase in dopamine release from said dopaminergic neuron, wherein said agent modulates: a T-type channel and/or an SK channel and/or the coupling of a T-type channel with an SK channel.

7. Use of an agent in the manufacture of a medicament, wherein said agent is capable of causing a dopaminergic neuron to enter bursting mode and/or of preventing it from leaving bursting mode, wherein said agent blocks: a T-type channel and/or an SK channel and/or the coupling of a T-type channel with an SK channel.

8. Use of an agent in the manufacture of a medicament, wherein said agent is capable of affecting the firing mode of a dopaminergic neuron that causes a beneficial change in dopamine release from said dopaminergic neuron, wherein said agent blocks: a T-type channel and/or an SK channel and/or the coupling of a T-type channel with an SK channel.

9. Use of an agent in the manufacture of a medicament, wherein said agent is capable of affecting the firing mode of a dopaminergic neuron that causes a beneficial increase in dopamine release from said dopaminergic neuron, wherein said agent blocks: a T-type channel and/or an SK channel and/or the coupling of a T-type channel with an SK channel.

10. A method of treatment comprising administering to a subject in need of same an agent, wherein said agent is capable of causing a dopaminergic neuron to enter bursting mode and/or of preventing it from leaving bursting mode.

11. A method of treatment comprising administering to a subject in need of same an agent, wherein said agent is capable of affecting the firing mode of a dopaminergic neuron that causes a beneficial change in dopamine release from said dopaminergic neuron.

12. A method of treatment comprising administering to a subject in need of same an agent, wherein said agent is capable of affecting the firing mode of a dopaminergic neuron that causes a beneficial increase in dopamine release from said dopaminergic neuron.

13. A method of treatment comprising administering to a subject in need of same an agent, wherein said agent is capable of causing a dopaminergic neuron to enter bursting mode and/or of preventing it from leaving bursting mode, wherein said agent modulates: a T-type channel and/or an SK channel and/or the coupling of a T-type channel with an SK channel.

14. A method of treatment comprising administering to a subject in need of same an agent, wherein said agent is capable of affecting the firing mode of a dopaminergic neuron that causes a beneficial change in dopamine release from said dopaminergic neuron, wherein said agent modulates: a T-type channel and/or an SK channel and/or the coupling of a T-type channel with an SK channel.

15. A method of treatment comprising administering to a subject in need of same an agent, wherein said agent is capable of affecting the firing mode of a dopaminergic neuron that causes a beneficial increase in dopamine release from said dopaminergic neuron, wherein said agent modulates: a T-type channel and/or an SK channel and/or the coupling of a T-type channel with an SK channel.

16. A method of treatment comprising administering to a subject in need of same an agent, wherein said agent is capable of causing a dopaminergic neuron to enter bursting mode and/or of preventing it from leaving bursting mode, wherein said agent blocks: a T-type channel and/or an SK channel and/or the coupling of a T-type channel with an SK channel.

17. A method of treatment comprising administering to a subject in need of same an agent, wherein said agent is capable of affecting the firing mode of a dopaminergic neuron that causes a beneficial change in dopamine release from said dopaminergic neuron, wherein said agent blocks: a T-type channel and/or an SK channel and/or the coupling of a T-type channel with an SK channel.

18. A method of treatment comprising administering to a subject in need of same an agent, wherein said agent is capable of affecting the firing mode of a dopaminergic neuron that causes a beneficial increase in dopamine release from said dopaminergic neuron, wherein said agent blocks: a T-type channel and/or an SK channel and/or the coupling of a T-type channel with an SK channel.

19. An assay method for identifying and/or improving the effect (such as by using the assay in a drug development program to improve efficacy) of an agent, wherein said agent is capable of causing a dopaminergic neuron to enter bursting mode and/or of preventing it from leaving bursting mode.

20. An assay method for identifying and/or improving the effect of an agent, wherein said agent is capable of affecting the firing mode of a dopaminergic neuron that causes a beneficial change in dopamine release from said dopaminergic neuron.

21. An assay method for identifying and/or improving the effect of an agent, wherein said agent is capable of affecting the firing mode of a dopaminergic neuron that causes a beneficial increase in dopamine release from said dopaminergic neuron.

22. An assay method for identifying and/or improving the effect of an agent, wherein said agent is capable of causing a dopaminergic neuron to enter bursting mode and/or of preventing it from leaving bursting mode, wherein said agent modulates: a T-type channel and/or an SK channel and/or the coupling of a T-type channel with an SK channel.

23. An assay method for identifying and/or improving the effect of an agent, wherein said agent is capable of affecting the firing mode of a dopaminergic neuron that causes a beneficial change in dopamine release from said dopaminergic neuron, wherein said agent modulates: a T-type channel and/or an SK channel and/or the coupling of a T-type channel with an SK channel.

24. An assay method for identifying and/or improving the effect of an agent, wherein said agent is capable of affecting the firing mode of a dopaminergic neuron that causes a beneficial increase in dopamine release from said dopaminergic neuron, wherein said agent modulates: a T-type channel and/or an SK channel and/or the coupling of a T-type channel with an SK channel.

25. An assay method for identifying and/or improving the effect of an agent, wherein said agent is capable of causing a dopaminergic neuron to enter bursting mode and/or of preventing it from leaving bursting mode, wherein said agent blocks: a T-type channel and/or an SK channel and/or the coupling of a T-type channel with an SK channel.

26. An assay method for identifying and/or improving the effect of an agent, wherein said agent is capable of affecting the firing mode of a dopaminergic neuron that causes a beneficial change in dopamine release from said dopaminergic neuron, wherein said agent blocks: a T-type channel and/or an SK channel and/or the coupling of a T-type channel with an SK channel.

27. An assay method for identifying and/or improving the effect of an agent, wherein said agent is capable of affecting the firing mode of a dopaminergic neuron that causes a beneficial increase in dopamine release from said dopaminergic neuron, wherein said agent blocks: a T-type channel and/or an SK channel and/or the coupling of a T-type channel with an SK channel.

28. An assay method for identifying and/or improving the effect (such as by using the assay in a drug development program to improve efficacy) of an agent, wherein said assay utilises: a T-type channel and/or an SK channel and/or the coupling of a T-type channel with an SK channel, wherein said agent is capable of causing a dopaminergic neuron to enter bursting mode and/or of preventing it from leaving bursting mode.

29. An assay method for identifying and/or improving the effect of an agent, wherein said assay utilises: a T-type channel and/or an SK channel and/or the coupling of a T-type channel with an SK channel, wherein said agent is capable of affecting the firing mode of a dopaminergic neuron that causes a beneficial change in dopamine release from said dopaminergic neuron.

30. An assay method for identifying and/or improving the effect of an agent, wherein said assay utilises: a T-type channel and/or an SK channel and/or the coupling of a T-type channel with an SK channel, wherein said agent is capable of affecting the firing mode of a dopaminergic neuron that causes a beneficial increase in dopamine release from said dopaminergic neuron.

31. An assay method for identifying and/or improving the effect of an agent, wherein said assay utilises: a T-type channel and/or an SK channel and/or the coupling of a T-type channel with an SK channel, wherein said agent is capable of causing a dopaminergic neuron to enter bursting mode and/or of preventing it from leaving bursting mode, wherein said agent modulates: a T-type channel and/or an SK channel and/or the coupling of a T-type channel with an SK channel.

32. An assay method for identifying and/or improving the effect of an agent, wherein said assay utilises: a T-type channel and/or an SK channel and/or the coupling of a T-type channel with an SK channel, wherein said agent is capable of affecting the firing mode of a dopaminergic neuron that causes a beneficial change in dopamine release from said dopaminergic neuron, wherein said agent modulates: a T-type channel and/or an SK channel and/or the coupling of a T-type channel with an SK channel.

33. An assay method for identifying and/or improving the effect of an agent, wherein said assay utilises: a T-type channel and/or an SK channel and/or the coupling of a T-type channel with an SK channel, wherein said agent is capable of affecting the firing mode of a dopaminergic neuron that causes a beneficial increase in dopamine release from said dopaminergic neuron, wherein said agent modulates: a T-type channel and/or an SK channel and/or the coupling of a T-type channel with an SK channel.

34. An assay method for identifying and/or improving the effect of an agent, wherein said assay utilises: a T-type channel and/or an SK channel and/or the coupling of a T-type channel with an SK channel, wherein said agent is capable of causing a dopaminergic neuron to enter bursting mode and/or of preventing it from leaving bursting mode, wherein said agent blocks: a T-type channel and/or an SK channel and/or the coupling of a T-type channel with an SK channel.

35. An assay method for identifying and/or improving the effect of an agent, wherein said assay utilises: a T-type channel and/or an SK channel and/or the coupling of a T-type channel with an SK channel, wherein said agent is capable of affecting the firing mode of a dopaminergic neuron that causes a beneficial change in dopamine release from said dopaminergic neuron, wherein said agent blocks: a T-type channel and/or an SK channel and/or the coupling of a T-type channel with an SK channel.

36. An assay method for identifying and/or improving the effect of an agent, wherein said assay utilises: a T-type channel and/or an SK channel and/or the coupling of a T-type channel with an SK channel, wherein said agent is capable of affecting the firing mode of a dopaminergic neuron that causes a beneficial increase in dopamine release from said dopaminergic neuron, wherein said agent blocks: a T-type channel and/or an SK channel and/or the coupling of a T-type channel with an SK channel.

37. A process comprising the steps of:
(a) performing the assay according to any one of the preceding claims;
(b) identifying one or more agents capable of modulating a T-type channel and/or an SK channel and/or the coupling of a T-type channel with an SK channel; and
(c) preparing a quantity of those one or more identified agents.

38. A method of treating a condition that can be alleviated with an increase in dopamine levels, preferably endogeneous dopamine levels comprising administering to a subject in need of same an agent; wherein the agent is capable of modulating a T-type channel and/or an SK channel and/or the coupling of a T-type channel with an SK channel in an in vitro assay method; wherein the in vitro assay method is the assay method defined in any one of the preceding claims.

39. Use of an agent in the preparation of a pharmaceutical composition for the treatment of a condition that can be alleviated with an increase in dopamine levels, preferably endogeneous dopamine levels, wherein the agent is capable of modulating a T-type channel and/or an SK channel and/or the coupling of a T-type channel with an SK channel in an in vitro assay method; wherein the in vitro assay method is the assay method defined in any one of the preceding claims.

40. An agent identified by the assay method according to any one of the preceding claims.

41. An agent according to claim 40 for use in medicine.

42. An agent according to claim 40 for use in treating a Parkinson's disease.

43. A T-type channel and/or an SK channel and/or the coupling of a T-type channel with an SK channel channel for use in medicine.

44. A modulator of a T-type channel and/or an SK channel and/or the coupling of a T-type channel with an SK channel channel for use in medicine.

45. A blocker of a T-type channel and/or an SK channel and/or the coupling of a T-type channel with an SK channel channel for use in medicine.

46. Use of a T-type channel and/or an SK channel and/or the coupling of a T-type channel with an SK channel channel for use in the preparation of a medicament for the treatment of a condition that can be alleviated with an increase in dopamine levels, preferably endogeneous dopamine levels, preferably wherein the condition is Parkinson's disease.

47. Use of a modulator of a T-type channel and/or an SK channel and/or the coupling of a T-type channel with an SK channel channel for use in the preparation of a medicament for the treatment of a condition that can be alleviated with an increase in dopamine levels, preferably endogeneous dopamine levels, preferably wherein the condition is Parkinson's disease.

48. Use of a blocker of a T-type channel and/or an SK channel and/or the coupling of a T-type channel with an SK channel channel for use in the preparation of a medicament for the treatment of a condition that can be alleviated with an increase in dopamine levels, preferably endogeneous dopamine levels, preferably wherein the condition is Parkinson's disease.

49. A diagnostic method for determining whether or not an individual has a neurdegenerative disorder comprising determining whether or not the individual has a defect affecting the structure and/or function of a T-type calcium channel and/or an SK potassium channel and/or the coupling thereof.

50. A method according to claim 49, wherein the method utilises a nucleic acid probe or primer to determine whether or not said individual has a genetic defect affecting the structure and/or function of a T-type calcium channel and/or an SK potassium channel and/or the coupling thereof.

51. The invention according to any one of the preceding claims wherein the SK potassium channel is an SK3 potassium channel.

52. The invention relating to any one of the preceding claims and substantially as described herein.
